(19) United States
(12) Patent Application Publication
(10) Pub. No.: US 2023/0002808 A1
(43) Pub. Date: Jan. 5, 2023

(54) METHODS FOR ANALYZING SPATIAL LOCATION OF NUCLEIC ACIDS

(71) Applicant: 10x Genomics, Inc., Pleasanton, CA (US)

(72) Inventor: Marco MIGNARDI, Sundbyberg (SE)

(21) Appl. No.: 17/853,256

(22) Filed: Jun. 29, 2022

Related U.S. Application Data
(60) Provisional application No. 63/217,126, filed on Jun. 30, 2021.

(51) Int. Cl. C12Q 1/6816; C12Q 1/6806; C12Q 1/683

(52) U.S. Cl. CPC C12Q 1/6816; C12Q 1/6806; C12Q 1/683; C12Q 2565/514

(57) ABSTRACT

The present disclosure provides, among other things, methods, compositions and kits for analyzing the presence and location of nucleic acids with respect to analytes in a biological sample, for example by hybridization of amplified nucleic acid probes. In some aspects, the present disclosure provides a method of assessing the spatial or geographical distribution of RNA in a biological sample.

### CROSS-REFERENCE TO RELATED APPLICATION

This application claims priority to U.S. Provisional Patent Application No. 63/217,126, filed Jun. 30, 2021, entitled “METHODS FOR ANALYZING SPATIAL LOCATION OF NUCLEIC ACIDS,” which is herein incorporated by reference in its entirety for all purposes.

### FIELD

The present disclosure relates in some aspects to methods and compositions for analyzing the presence and location of target nucleic acids, such as mRNA molecules, in a biological sample (e.g., in situ), in connection with an analyte in the biological sample.

### BACKGROUND

Methods are available for analyzing nucleic acids present in a biological sample, such as a cell or a tissue. Current methods for analyzing target nucleic acids present in a biological sample, for example for in situ analysis, can be limited in that spatial location of target nucleic acids, such as mRNA molecules, particularly in connection with another analyte, is difficult to determine. Further, current methods can have low sensitivity and specificity, limited plexity, and can be biased, time-consuming, labor-intensive, and/or error-prone. Improved methods for analyzing nucleic acids, particularly spatial location of nucleic acids, and other analytes, are needed. Provided herein are methods, compositions, and kits that meet such and other needs.

### BRIEF SUMMARY

Provided herein are methods of analyzing a biological sample, comprising: contacting a biological sample with: a labeling agent comprising an analyte-binding moiety conjugated to a reporter oligonucleotide, and a nucleic acid probe comprising a cassette comprising (1) a restriction site, (2) a nucleic acid capture sequence, and (3) a barcode sequence, wherein the nucleic acid probe hybridizes to the reporter oligonucleotide; amplifying the nucleic acid probe to generate a concatemer of monomer sequences, wherein each monomer sequence comprises sequences that are complementary to the cassette; generating a plurality of monomers by cleaving the concatemer at the restriction site, wherein a monomer of the plurality of monomers comprises a complement of the nucleic acid capture sequence at the 3′ end; and allowing the monomer to hybridize to a target nucleic acid in the biological sample via the complement.

Provided herein are methods of analyzing a biological sample, comprising: contacting a biological sample with: a labeling agent comprising an analyte-binding moiety conjugated to a reporter oligonucleotide, and a nucleic acid probe comprising (1) a restriction site, (2) a nucleic acid capture sequence, and (3) a barcode sequence, wherein the nucleic acid probe hybridizes to the reporter oligonucleotide; amplifying the nucleic acid probe to generate a concatemer of monomer sequences, wherein each monomer sequence comprises sequences that are complementary to the nucleic acid probe; generating a plurality of monomers by cleaving the concatemer at the restriction site, wherein a monomer of the plurality of monomers comprises a complement of the nucleic acid capture sequence at the 3′ end; and allowing the monomer to hybridize to a target nucleic acid in the biological sample via the complement.

In any of the embodiments herein, the method can also involve, before generating a plurality of monomers by cleaving the concatemer, detecting the concatemer or a portion thereof at a spatial location of the biological sample.

In any of the embodiments herein, the nucleic acid capture sequence is or can comprise a plurality of adenine (A) polynucleotides, a plurality of thymine (T) nucleotides, or a gene specific sequence.

In any of the embodiments herein, the cleaving of the concatemer at the restriction site can comprise contacting the biological sample with a restriction endonuclease that recognizes the restriction site. In some of any of the provided embodiments, the cleaving can also involve a restriction-site oligonucleotide comprising nucleic acid sequences complementary to the restriction site.

In any of the embodiments herein, the target nucleic acid can comprise an RNA molecule present in the biological sample or a sequence thereof, a reverse transcription product thereof, a complement thereof, a hybridization product thereof, a ligation product thereof, an extension product thereof, a replication product thereof, and/or an amplification product thereof.

In any of the embodiments herein, the target nucleic acid can comprise a messenger RNA (mRNA) molecule present at or near a labeling agent in the biological sample or a reverse transcription product thereof. In any of the embodiments herein, the target nucleic acid can comprise an individual mRNA molecule transcribed from a particular gene, and the nucleic acid capture sequence is or can comprise a gene specific sequence. In any of the embodiments herein, the target nucleic acid can comprise a reverse transcription product of an mRNA molecule. In any of the embodiments herein, the reverse transcription product can be a complementary DNA (cDNA).

In any of the embodiments herein, the method can also involve, before allowing the monomer to hybridize to a target nucleic acid in the biological sample via the complement, generating a reverse transcription product of an mRNA molecule present in the biological sample. In any of the embodiments herein, the reverse transcription product can be a second-strand cDNA. In any of the embodiments herein, the second-strand cDNA can be generated using a template-switch oligonucleotide (TSO).

In any of the embodiments herein, the target nucleic acid can comprises an mRNA molecule that comprises a polyA tail. In any of the embodiments herein, the nucleic acid capture sequence is or can comprise a plurality of adenine (A) nucleotides. In any of the embodiments herein, the complement in the monomer can hybridize to the polyA tail of the mRNA molecule. In any of the embodiments herein, the plurality of adenine (A) nucleotides can comprise at least 2, 3, 4, 5, 6, 7, 8, 9, 10, 15, 20, 25, 30, 35, 40, 45, 50, 60, 70, 80, 90, 100 or more adenine (A) nucleotides.

In any of the embodiments herein, the restriction endonuclease can be selected from among DraI, MseI, TaqI-v2 and XbaI. In any of the embodiments herein, the restriction endonuclease can be DraI.

In any of the embodiments herein, the method can also involve generating a library of tagged complementary DNA (cDNA) by performing reverse transcription of the target nucleic acids using the complement in the monomer as a reverse transcription primer.

In any of the embodiments herein, the reverse transcription product can be a circularized cDNA. In any of the embodiments herein, the circularized cDNA can be generated by reverse transcription of an mRNA molecule in the biological sample, and circularizing the reverse transcription product. In any of the embodiments herein, the circularizing can involve a single-stranded DNA ligase that catalyzes intramolecular ligation.

In any of the embodiments herein, the nucleic acid capture sequence is or can comprise a plurality of thymine (T) nucleotides. In any of the embodiments herein, the complement in the monomers can hybridize to sequences present in the reverse transcription product. In any of the embodiments herein, the plurality of thymine (T) nucleotides can comprise at least 2, 3, 4, 5, 6, 7, 8, 9, 10, 15, 20, 25, 30, 35, 40, 45, 50, 60, 70, 80, 90, 100 or more thymine (T) nucleotides.

In any of the embodiments herein, the restriction endonuclease can be selected from among AcII, AgeI, BgIII, HindIII, SpeI, MluI, and PciI.

In any of the embodiments herein, the method can also involve, after allowing the monomer to hybridize to a target nucleic acid in the biological sample via the complement, generating a library of tagged cDNA by priming the extension of the cDNA using the complement in the monomer as an extension primer. In any of the embodiments herein, the method can also involve analyzing the library of tagged cDNA by sequencing the tagged cDNAs or a portion thereof.

In some aspects, provided herein are methods of analyzing a biological sample that involves: contacting a biological sample with: a labeling agent comprising an analyte-binding moiety conjugated to a reporter oligonucleotide, and a nucleic acid probe comprising a cassette comprising (1) a restriction site, (2) a plurality of adenine (A) nucleotides, (3) a barcode sequence comprising an analyte-binding moiety-specific barcode sequence and a spatial barcode sequence and (4) an adapter sequence, wherein the nucleic acid probe hybridizes to the reporter oligonucleotide; amplifying the nucleic acid probe to generate a concatemer of monomer sequences by rolling circle amplification (RCA), wherein each monomer sequence comprises sequences that are complementary to the cassette; detecting the concatemer or a portion thereof at a spatial location of the biological sample by in situ sequencing and/or in situ hybridization; generating a plurality of monomers by cleaving the concatemer at the restriction site, wherein a monomer of the plurality of monomers comprises a complement of the nucleic acid capture sequence at the 3′ end that comprises an oligo dT sequence; and allowing the monomer to hybridize to a target nucleic acid via the complement, wherein the target nucleic acid comprises an mRNA molecule present at or near the labeling agent in the biological sample; generating a library of tagged complementary DNA (cDNA) by performing reverse transcription of the target nucleic acids using the complement in the monomers as a reverse transcription primer; and analyzing the library of tagged cDNA by sequencing the tagged cDNAs or a portion thereof.

In some aspects, provided herein are methods of analyzing a biological sample that involves: contacting a biological sample with: a labeling agent comprising an analyte-binding moiety conjugated to a reporter oligonucleotide, and a nucleic acid probe comprising (1) a restriction site, (2) a plurality of adenine (A) nucleotides, (3) a barcode sequence comprising an analyte-binding moiety-specific barcode sequence and a spatial barcode sequence and (4) an adapter sequence, wherein the nucleic acid probe hybridizes to the reporter oligonucleotide; amplifying the nucleic acid probe to generate a concatemer of monomer sequences by rolling circle amplification (RCA), wherein each monomer sequence comprises sequences that are complementary to the nucleic acid probe; detecting the concatemer or a portion thereof at a spatial location of the biological sample by in situ sequencing and/or in situ hybridization; generating a plurality of monomers by cleaving the concatemer at the restriction site, wherein a monomer of the plurality of monomers comprises a complement of the nucleic acid capture sequence at the 3′ end that comprises an oligo dT sequence; and allowing the monomer to hybridize to a target nucleic acid via the complement, wherein the target nucleic acid comprises an mRNA molecule present at or near the labeling agent in the biological sample; generating a library of tagged complementary DNA (cDNA) by performing reverse transcription of the target nucleic acids using the complement in the monomers as a reverse transcription primer; and analyzing the library of tagged cDNA by sequencing the tagged cDNAs or a portion thereof.

In some aspects, provided herein are methods of analyzing a biological sample that involves: generating, in a biological sample a target nucleic acid that comprises a circular cDNA that is complementary to an mRNA molecule in the biological sample, by reverse transcription of the mRNA molecule, and circularizing the cDNA; contacting the biological sample with: a labeling agent comprising an analyte-binding moiety conjugated to a reporter oligonucleotide, and a nucleic acid probe comprising a cassette comprising (1) a restriction site, (2) a plurality of thymine (T) nucleotides, (3) a barcode sequence comprising an analyte-binding moiety-specific barcode sequence and a spatial barcode sequence and (4) an adapter sequence, wherein the nucleic acid probe hybridizes to the reporter oligonucleotide; amplifying the nucleic acid probe to generate a concatemer of monomer sequences by rolling circle amplification (RCA), wherein each monomer sequence comprises sequences that are complementary to the cassette; detecting the concatemer or a portion thereof at a spatial location of the biological sample by in situ sequencing and/or in situ hybridization; generating a plurality of monomers by cleaving the concatemer at the restriction site, wherein a monomer of the plurality of monomers comprises a complement of the nucleic acid capture sequence at the 3′ end that comprises an oligo dA sequence; allowing the monomer to hybridize to the target nucleic acid via the complement; (generating a library of tagged cDNA by priming the extension of the circularized cDNA with the complement in the monomer; and analyzing the library of tagged cDNA by sequencing the tagged cDNAs or a portion thereof.

In some aspects, provided herein is a method of analyzing a biological sample that involves: generating, in a biological sample a target nucleic acid that comprises a second-strand cDNA of an mRNA molecule in the biological sample, by reverse transcription of the mRNA molecule and generating the second-strand cDNA using a template-switch oligonucleotide (TSO); contacting a biological sample with: a labeling agent comprising an analyte-binding moiety conjugated to a reporter oligonucleotide, and a nucleic acid probe comprising a cassette comprising (1) a restriction site, (2) a plurality of adenine (A) nucleotides, (3) a barcode sequence comprising an analyte-binding moiety-specific barcode sequence and a spatial barcode sequence and (4) an adapter sequence, wherein the nucleic acid probe hybridizes to the reporter oligonucleotide; amplifying the nucleic acid probe to generate a concatemer of monomer sequences by rolling circle amplification (RCA), wherein each monomer sequence comprises sequences that are complementary to the cassette; (b′) detecting the concatemer or a portion thereof at a spatial location of the biological sample by in situ sequencing and/or in situ hybridization; generating a plurality of monomers by cleaving the concatemer at the restriction site, wherein a monomer of the plurality of monomers comprises a complement of the nucleic acid capture sequence at the 3′ end that comprises an oligo dT sequence; allowing the monomer to hybridize to the target nucleic acid via the complement; generating a library of tagged cDNA by priming the extension of the second strand cDNA with the complement in the monomer; and analyzing the library of tagged cDNA by sequencing the tagged cDNAs or a portion thereof.

In some aspects, provided herein is a method of analyzing a biological sample that involves: generating, in a biological sample a target nucleic acid that comprises a second-strand cDNA of an mRNA molecule in the biological sample, by reverse transcription of the mRNA molecule and generating the second-strand cDNA using a template-switch oligonucleotide (TSO); contacting a biological sample with: a labeling agent comprising an analyte-binding moiety conjugated to a reporter oligonucleotide, and a nucleic acid probe comprising a (1) a restriction site, (2) a plurality of adenine (A) nucleotides, (3) a barcode sequence comprising an analyte-binding moiety-specific barcode sequence and a spatial barcode sequence and (4) an adapter sequence, wherein the nucleic acid probe hybridizes to the reporter oligonucleotide; amplifying the nucleic acid probe to generate a concatemer of monomer sequences by rolling circle amplification (RCA), wherein each monomer sequence comprises sequences that are complementary to the nucleic acid probe; (b′) detecting the concatemer or a portion thereof at a spatial location of the biological sample by in situ sequencing and/or in situ hybridization; generating a plurality of monomers by cleaving the concatemer at the restriction site, wherein a monomer of the plurality of monomers comprises a complement of the nucleic acid capture sequence at the 3′ end that comprises an oligo dT sequence; allowing the monomer to hybridize to the target nucleic acid via the complement; generating a library of tagged cDNA by priming the extension of the second strand cDNA with the complement in the monomer; and analyzing the library of tagged cDNA by sequencing the tagged cDNAs or a portion thereof.

In any of the embodiments herein, the barcode sequence can comprise an analyte-binding moiety-specific barcode sequence. In any of the embodiments herein, the analyte-binding moiety-specific barcode sequence can uniquely identify the analyte-binding moiety among the labeling agent comprising different analyte-binding moieties. In any of the embodiments herein, the barcode sequence can further comprise a spatial barcode sequence. In any of the embodiments herein, the spatial barcode sequence can uniquely identify a single labeling agent molecule. In any of the embodiments herein, the spatial barcode sequence can correspond to the position of the single labeling agent molecule in the biological sample.

In any of the embodiments herein, the amplification to generate a concatemer can be carried out using rolling circle amplification (RCA). In any of the embodiments herein, the RCA can be performed using a circularized nucleic acid probe as a template and the reporter oligonucleotide as a primer. In any of the embodiments herein, the RCA can be performed using a circularized nucleic acid probe as a template and a second oligonucleotide as a primer.

In any of the embodiments herein, the concatemer can be generated in situ. In any of the embodiments herein, the concatemer can be between about 1 and about 15 kilobases, between about 15 and about 25 kilobases, between about 25 and about 35 kilobases, between about 35 and about 45 kilobases, or between about 45 and about 50 kilobases in length. In any of the embodiments herein, the concatemer can form a nanoball having a diameter between about 0.1 μm and about 3 μm.

In any of the embodiments herein, detecting the concatemer or a portion thereof can comprise determining a sequence of the barcode sequence or a complementary sequence thereof or an amplified sequence thereof. In any of the embodiments herein, detecting the concatemer or a portion thereof can comprise in situ sequencing and/or in situ hybridization. In any of the embodiments herein, the in situ sequencing can comprise sequencing by ligation, sequencing by hybridization, sequencing by synthesis, and/or sequencing by binding. In any of the embodiments herein, the in situ hybridization can comprise sequential fluorescent in situ hybridization.

In any of the embodiments herein, detecting the concatemer or a portion thereof can comprise imaging the biological sample. In any of the embodiments herein, imaging can comprise fluorescent microscopy.

In any of the embodiments herein, the reverse transcription of the mRNA can be performed in the presence of a modified nucleotide. In any of the embodiments herein, the modified nucleotide can comprise a cross-linkable nucleotide. In any of the embodiments herein, the modified nucleotide can be selected from among a halogenated base, an azide-modified base, an aminoallyl dUTP, an octadiynyl dU, a thiol-modified base, a biotin-modified base, or a combination thereof.

In any of the embodiments herein, the method can also involve crosslinking the modified nucleotide to the sample, a substrate, and/or a matrix, thereby crosslinking the cDNA to the sample, the substrate, and/or the matrix. In any of the embodiments herein, the cassette can further comprise an adapter sequence.

In any of the embodiments herein, the sequencing the tagged cDNAs or a portion thereof can comprise direct sequencing or indirect sequencing. In any of the embodiments herein, the method can also involve, prior to the sequencing, ligating or adding a second adapter sequence the tagged cDNA.

In any of the embodiments herein, the tagged cDNA in the library or a portion thereof can be amplified prior to the sequencing. In any of the embodiments herein, the portion of the tagged cDNA that is sequenced can comprise at least the barcode sequence and a portion of the target nucleic acid or complement thereof. In any of the embodiments herein, the portion of the tagged cDNA that is sequenced can comprise the spatial barcode sequence.

In any of the embodiments herein, the analyte-binding moiety may be a protein, a peptide, an antibody or an epitope binding fragment thereof, a lipophilic moiety, a cell surface receptor binding molecule, a receptor ligand, a small molecule, a bi-specific antibody, a bi-specific T-cell engager, a T-cell receptor engager, a B-cell receptor engager, a pro-body, an aptamer, a monobody, an affimer, a darpin, and a protein scaffold, or any combination thereof. In any of the embodiments herein, the analyte-binding moiety may be an antibody or an epitope binding fragment thereof.

In any of the embodiments herein, the biological sample may be a processed or cleared biological sample. In any of the embodiments herein, the biological sample can be a tissue sample. In any of the embodiments herein, the tissue sample can be a tissue slice between about 1 μm and about 50 μm in thickness. In any of the embodiments herein, the tissue slice can be between about 5 μm and about 35 μm in thickness.

In any of the embodiments herein, the biological sample may be embedded in a hydrogel. In any of the embodiments herein, the biological sample may not be embedded in a hydrogel. In any of the embodiments herein, the biological sample can be fixed. In any of the embodiments herein, the method can also involve fixing the biological sample. Also provided herein are kits. In any of the embodiments herein, the kits can comprise: a labeling agent comprising an analyte-binding moiety conjugated to a reporter oligonucleotide; and a nucleic acid probe comprising a cassette comprising (1) a restriction site, (2) a nucleic acid capture sequence, and (3) a barcode sequence, wherein the nucleic acid probe hybridizes to the reporter oligonucleotide. In any of the embodiments herein, the kit also can comprise instructions for performing any of the methods provided herein.

In any of the embodiments herein, the kit can also comprise a reverse transcriptase.

In any of the embodiments herein, the kit can also comprise a restriction endonuclease. In any of the embodiments herein, the restriction endonuclease may be selected from among DraI, MseI, TaqI-v2 and XbaI. In some embodiments, the restriction endonuclease is DraI. In any of the embodiments herein, the restriction endonuclease may be selected from among AcII, AgeI, BgIII, HindIII, SpeI, MluI, and PciI.

In any of the embodiments herein, the kit can also comprise a template-switch oligonucleotide (TSO).

Also provided herein are compositions. In any of the embodiments herein, the compositions can comprise: a labeling agent comprising an analyte-binding moiety conjugated to a reporter oligonucleotide; a nucleic acid probe comprising a cassette comprising (1) a restriction site, (2) a nucleic acid capture sequence, and (3) a barcode sequence, wherein the nucleic acid probe hybridizes to the reporter oligonucleotide; and a biological sample comprising a target nucleic acid. In any of the embodiments herein, the compositions can comprise: a labeling agent comprising an analyte-binding moiety conjugated to a reporter oligonucleotide; a nucleic acid probe comprising (1) a restriction site, (2) a nucleic acid capture sequence, and (3) a barcode sequence, wherein the nucleic acid probe hybridizes to the reporter oligonucleotide; and a biological sample comprising a target nucleic acid. In any of the embodiments herein, the composition can also comprise an amplification product of the nucleic acid probe that comprises a concatemer of monomer sequences, wherein each monomer sequence can comprise sequences that are complementary to the cassette.

### BRIEF DESCRIPTION OF THE DRAWINGS

FIG. 1 depicts an exemplary analyte-binding moiety conjugated to a reporter oligonucleotide, and components of an exemplary nucleic acid probe. The nucleic acid probe comprises a cassette comprising a restriction site, a nucleic acid capture sequence, and a barcode sequence. In FIG. 1, the nucleic acid probe hybridizes to the reporter oligonucleotide to amplify the nucleic acid probe (e.g. by rolling circle amplification) and generate a concatemer (e.g., rolling circle amplification product (RCP)) of monomer sequences.

FIG. 2 depicts the cleavage of the concatemer at the restriction site resulting in cleaved RCP products (monomers). In FIG. 2, the monomers can diffuse into surrounding regions of the biological sample (e.g., a tissue sample) and hybridize to nearby target nucleic acids via the complement of the nucleic acid capture sequence (e.g., oligo dT). The hybridization can prime a reverse transcription of the target nucleic acid (e.g., mRNA), generating a tagged cDNA sequence.

FIG. 3 depicts an exemplary method in which: first, the mRNA molecule is reverse transcribed and the resulting cDNA is circularized in a biological sample (e.g. a tissue sample); second, a labeling agent, comprising an analyte-binding moiety conjugated to a reporter oligonucleotide, and a nucleic acid probe comprising a cassette comprising a restriction site, a nucleic acid capture sequence, and a barcode sequence, is added; and third, the nucleic acid probe is amplified to generate a concatemer (e.g., RCP) of monomer sequences by rolling circle amplification, and cleaved to generate monomers that could prime nearby circularized cDNA molecules to generate tagged cDNA.

### DETAILED DESCRIPTION

Provided herein in some aspects are methods for analyzing a biological sample in situ, for example assessing one or more target nucleic acid(s) (for example, messenger RNAs) and/or one or more analyte(s) present in a biological sample. Also provided are polynucleotides, compositions, kits, systems and devices for use in accordance with the provided methods. In some aspects, the provided methods are quantitative and preserves the spatial information within a tissue sample without physically isolating cells or using homogenates. In some aspects, the provided embodiments can be employed for in situ spatial analysis, detection, quantitation and/or sequencing of the target nucleic acid(s), analyte(s) and/or in some aspects, the spatial or geographic location or relative location of the target nucleic acid(s) and analyte(s).

All publications, comprising patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. Overview

Provided herein are methods, kits, compositions and systems for analyzing a biological sample, such as for in situ analysis of one or more target nucleic acid(s) and/or one or more analyte(s) in a cell in an intact tissue sample, that involves the use of a labeling agent and a nucleic acid probe. In some aspects, the provided methods involve the generation of a concatemer of monomer sequences by amplification of the nucleic acid probe, and generation of a plurality of monomers by cleaving the concatemer, e.g., via restriction endonuclease digestion. In some aspects, the provided embodiments can be applied to detect, image, quantitate or determine the sequence of target nucleic acid(s), for example, to determine the spatial or geographical location of target nucleic acid(s). In some aspects, the provided embodiments can be applied to detect, image, quantitate or determine the spatial location of one or more analyte(s) also present in the biological sample. In some aspects, the provided embodiments can be applied to determine the spatial or geographic location of target nucleic acid(s) in relation to the spatial or geographic location of the analyte(s). In some aspects, the provided methods can be applied for various applications, including for in situ analysis, including in situ detection and/or sequencing of target nucleic acids and multiplexed nucleic acid analysis, and in situ detection of the analyte(s).

In some aspects, existing methods for assessing nucleic acids in a biological sample, particularly for in situ analysis, can have certain limitations, such as low sensitivity or low efficiency of detection, and/or the need to design sequence specific probes to determine the spatial location of nucleic acids. In some aspects, existing methods may also have limitations such as low sensitivity or specificity, limited plexity, biases, high costs and/or are time-consuming, labor-intensive, and/or error-prone. In addition, based on existing methods, it is difficult to assess one or more analyte(s), such as one or more proteins or other analytes, in addition to the nucleic acids, or their relative spatial locations or relative spatial distribution of nucleic acids in a biological sample.

In some aspects, to assess the spatial or geographic location of nucleic acids and other analytes, for example to investigate protein-RNA interactions, knowing the distribution of nucleic acids, such as RNA, at a particular location or surrounding a particular other analyte, such as a particular protein, is useful. In some aspects, there is a need for methods for detecting in situ one or more analyte(s) such as a protein, and surveying or cataloging nucleic acids that are physically near or the analyte(s), without the need to design sequence specific probes for each nucleic acid.

Provided are methods, polynucleotides, compositions, kits, devices and systems, that overcome some or all of these limitations. For instance, the methods described herein can provide valuable information regarding the proximity of particular nucleic acids, e.g., specific mRNAs, to particular analytes, e.g., specific proteins, in situ, which can be important for investigation of protein-RNA interaction or presence of specific RNA at particular locations. Provided herein are methods that allow for the determination of the spatial or geographic distribution of nucleic acids in a biological sample. The methods provided herein allow for the recognition of the geographical distribution of RNA in a biological sample. In some aspects, the provided methods involve the use of a labeling agent that comprises an analyte-binding moiety conjugated to a reporter oligonucleotide and a nucleic acid probe. The nucleic acid probe comprises specific components, such as a restriction site, a nucleic acid capture sequence, and a barcode sequence. In some aspects, the nucleic acid probe can be amplified and cleaved to generate a plurality of monomers, which can be hybridized to nucleic acids that are near or proximate to the analyte. The provided embodiments involve assessing particular analytes, e.g., specific proteins, via the analyte-binding moiety (for example, an antibody), and nucleic acids that are nearby, by hybridization with the nucleic acid capture sequence of the amplified and cleaved monomers.

In some aspects, the provided embodiments offer advantages, including the ability to survey nucleic acids that are physically or geographically near particular analytes, without the need to know a priori the sequence of the nucleic acids or designing specific probes to target particular nucleic acid sequences. The provided embodiments also offer an ability to concurrently or sequentially detect or analyze multiple different components and aspects of the biological samples and/or intermediaries or products of the methods, including the analyte(s), nucleic acid(s), nucleic acid probe(s) and/or amplified products, and combine or synthesize the information to determine their relative physical or spatial location. For example, the provided embodiments allow for the determination of the proximity of nucleic acids (e.g., mRNA) in relation to a specific analyte (e.g., a specific protein). In addition, the provided embodiments allow the analysis of the biological sample in a highly multiplexed fashion and in situ, for example, while the nucleic acids and/or analytes generally maintain their original location in the sample. The provided embodiments also offer the flexibility of using in situ detection or analysis methods or high-throughput sequencing methods, or combining the information from both, to determine the spatial or geographic location of the assessed components in the biological sample. Thus, the provided methods, compositions, kits, devices and systems provide numerous advantages compared to existing analysis methods.

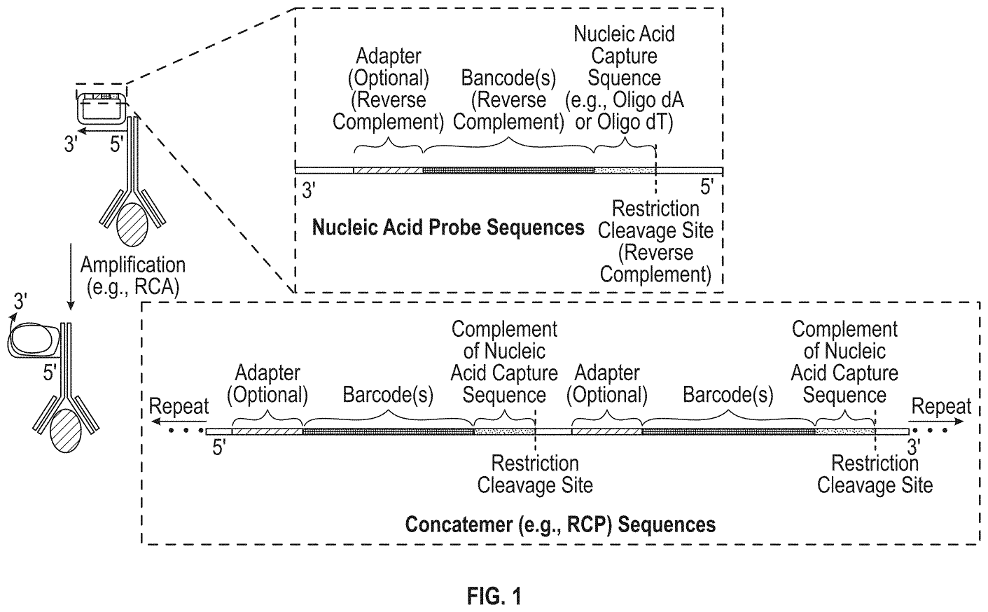

In some aspects, the provided embodiments involve contacting a biological sample with a labeling agent comprising an analyte-binding moiety conjugated to a reporter oligonucleotide. In some aspects, the provided embodiments involve contacting a biological sample with a nucleic acid probe. In some embodiments, the nucleic acid probe comprises a cassette comprising (1) a restriction site, (2) a nucleic acid capture sequence, and (3) a barcode sequence, wherein the nucleic acid probe hybridizes to the reporter oligonucleotide. Exemplary nucleic acid probes are illustrated in FIG. 1. In some aspects, the provided embodiments involve amplifying the nucleic acid probe to generate a concatemer of monomer sequences. In some aspects, each monomer sequence comprises sequences that are complementary to the cassette. In some aspects, the provided embodiments involve generating a plurality of monomers by cleaving the concatemer at the restriction site. In some aspects, a monomer of the plurality of monomers comprises a complement of the nucleic acid capture sequence at the 3′ end. In some aspects, the provided embodiments involve allowing the monomer to hybridize to a target nucleic acid in the biological sample via the complement.

In some aspects, amplification of the nucleic acid probe results in the production of a concatemer (e.g., by rolling circle amplification (RCA), to produce a rolling circle amplification product (RCP) of monomer sequences which are complementary to the sequence of the probe. In some embodiments, the concatemer (e.g., RCP) can then be enzymatically cleaved into monomers at the cleavage site. Following cleavage, a complement of the nucleic acid capture sequence (e.g., an oligo dT or an oligo dA sequence) is present at the 3′ end of each of the monomers. In some embodiments, the monomers can diffuse into the surrounding area of the biological sample, in which the complement of the nucleic acid capture sequence, at the 3′ end of the monomer hybridizes to and captures the target nucleic acid (e.g., polyA tail of the mRNA). Exemplary concatemers (such as RCP) are illustrated in FIG. 1.

In some aspects, the method also involves generating a library of tagged complementary DNA (cDNA) by performing reverse transcription of the target nucleic acids (e.g., mRNA present in the biological sample and hybridized to a portion of the monomers) using the complement in the monomer as a reverse transcription primer. In some embodiments, a tagged cDNA library is constructed and detected or sequenced in situ, or via high throughput sequencing methods. In some aspects, one or more barcodes that are present in the monomer can be used to identify the analyte (e.g., via specifying particular analyte-binding moieties) and/or a particular spatial location (e.g., spatial barcode can specify particular location of the biological sample).

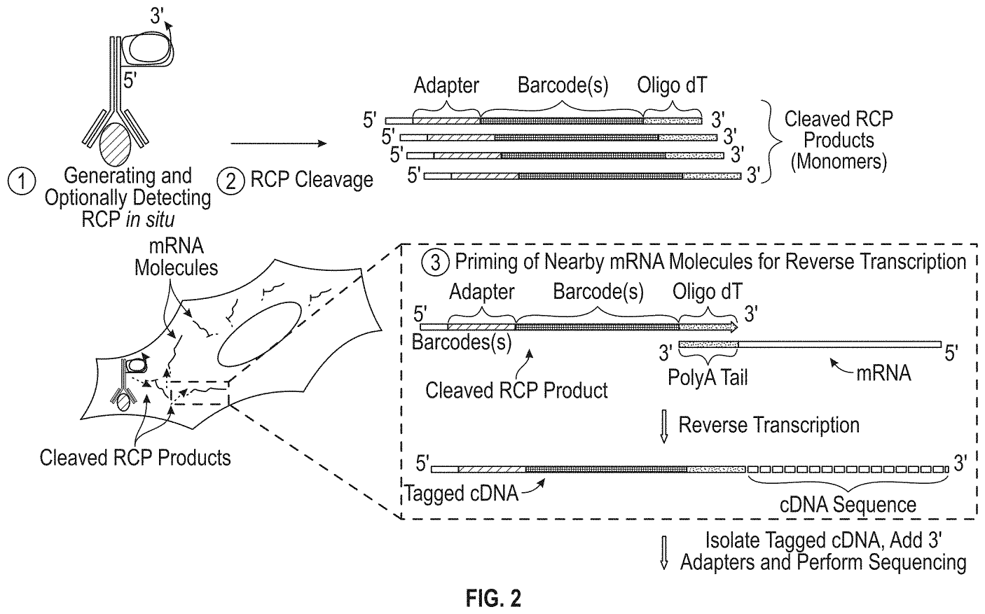

In some aspects, the provided methods involve contacting a biological sample with: a labeling agent comprising an analyte-binding moiety conjugated to a reporter oligonucleotide, and a nucleic acid probe comprising a cassette comprising (1) a restriction site, (2) a plurality of adenine (A) nucleotides, (3) a barcode sequence comprising an analyte-binding moiety-specific barcode sequence and a spatial barcode sequence and (4) an adapter sequence, wherein the nucleic acid probe hybridizes to the reporter oligonucleotide; amplifying the nucleic acid probe to generate a concatemer of monomer sequences by rolling circle amplification (RCA), wherein each monomer sequence comprises sequences that are complementary to the cassette; detecting the concatemer or a portion thereof at a spatial location of the biological sample by in situ sequencing and/or in situ hybridization; generating a plurality of monomers by cleaving the concatemer at the restriction site, wherein a monomer of the plurality of monomers comprises a complement of the nucleic acid capture sequence at the 3′ end that comprises an oligo dT sequence; and allowing the monomer to hybridize to a target nucleic acid via the complement, wherein the target nucleic acid comprises an mRNA molecule present at or near the labeling agent in the biological sample; generating a library of tagged complementary DNA (cDNA) by performing reverse transcription of the target nucleic acids using the complement in the monomers as a reverse transcription primer; and analyzing the library of tagged cDNA by sequencing the tagged cDNAs or a portion thereof. Exemplary of such methods are illustrated in FIG. 2.

In some aspects, the embodiments can involve, before allowing the monomer to hybridize to a target nucleic acid in the biological sample via the complement, generating a reverse transcription product of mRNA molecules present in the biological sample.

In some embodiments, the reverse transcription product can be a second-strand cDNA, or a circularized cDNA. In some embodiments, the second-strand cDNA is generated using a template-switch oligonucleotide (TSO). In some embodiments, the circularized cDNA is generated by reverse transcription of an mRNA molecule in the biological sample, and circularizing the reverse transcription product.

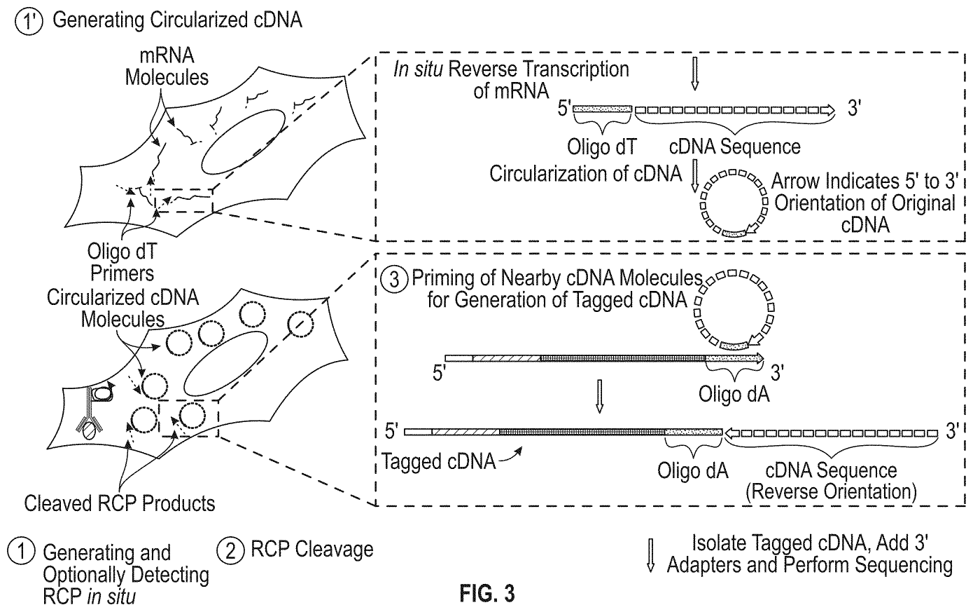

In some aspects, the provided methods involve generating, in a biological sample a target nucleic acid that comprises a circular cDNA that is complementary to an mRNA molecule in the biological sample, by reverse transcription of the mRNA molecule, and circularizing the cDNA; contacting the biological sample with: a labeling agent comprising an analyte-binding moiety conjugated to a reporter oligonucleotide, and a nucleic acid probe comprising a cassette comprising (1) a restriction site, (2) a plurality of thymine (T) nucleotides, (3) a barcode sequence comprising an analyte-binding moiety-specific barcode sequence and a spatial barcode sequence and (4) an adapter sequence, wherein the nucleic acid probe hybridizes to the reporter oligonucleotide; amplifying the nucleic acid probe to generate a concatemer of monomer sequences by rolling circle amplification (RCA), wherein each monomer sequence comprises sequences that are complementary to the cassette; detecting the concatemer or a portion thereof at a spatial location of the biological sample by in situ sequencing and/or in situ hybridization; generating a plurality of monomers by cleaving the concatemer at the restriction site, wherein a monomer of the plurality of monomers comprises a complement of the nucleic acid capture sequence at the 3′ end that comprises an oligo dA sequence; allowing the monomer to hybridize to the target nucleic acid via the complement; (generating a library of tagged cDNA by priming the extension of the circularized cDNA with the complement in the monomer; and analyzing the library of tagged cDNA by sequencing the tagged cDNAs or a portion thereof. Exemplary of such methods are illustrated in FIG. 3.

In some aspects, provided herein is a method of analyzing a biological sample that involves: generating, in a biological sample a target nucleic acid that comprises a second-strand cDNA of an mRNA molecule in the biological sample, by reverse transcription of the mRNA molecule and generating the second-strand cDNA using a template-switch oligonucleotide (TSO); contacting a biological sample with: a labeling agent comprising an analyte-binding moiety conjugated to a reporter oligonucleotide, and a nucleic acid probe comprising a cassette comprising (1) a restriction site, (2) a plurality of adenine (A) nucleotides, (3) a barcode sequence comprising an analyte-binding moiety-specific barcode sequence and a spatial barcode sequence and (4) an adapter sequence, wherein the nucleic acid probe hybridizes to the reporter oligonucleotide; amplifying the nucleic acid probe to generate a concatemer of monomer sequences by rolling circle amplification (RCA), wherein each monomer sequence comprises sequences that are complementary to the cassette; (b′) detecting the concatemer or a portion thereof at a spatial location of the biological sample by in situ sequencing and/or in situ hybridization; generating a plurality of monomers by cleaving the concatemer at the restriction site, wherein a monomer of the plurality of monomers comprises a complement of the nucleic acid capture sequence at the 3′ end that comprises an oligo dT sequence; allowing the monomer to hybridize to the target nucleic acid via the complement; generating a library of tagged cDNA by priming the extension of the second strand cDNA with the complement in the monomer; and analyzing the library of tagged cDNA by sequencing the tagged cDNAs or a portion thereof.

### II. Labeling Agents and Nucleic Acid Probes

The methods disclosed herein comprise contacting a biological sample with a labeling agent comprising an analyte-binding moiety conjugated to a reporter oligonucleotide, and a nucleic acid probe. In certain embodiments, provided herein are methods for analyzing nucleic acids present in a biological sample, such as a cell or a tissue, using one or more labeling agents. In some embodiments, a labeling agent may include an analyte-binding moiety that interacts with an analyte (e.g., a protein). In some embodiments, the labeling agents can comprise a reporter oligonucleotide that is indicative of the analyte interacting with the labeling agent. In some cases, the sample contacted by the labeling agent is further contacted with a nucleic acid probe (e.g., circularizable probe such as a padlock probe), that hybridizes to a reporter oligonucleotide of the labeling agent, in order to identify the analyte associated with the labeling agent. In some embodiments, the labeling agent comprises an analyte-binding moiety and a labeling agent barcode domain comprising one or more barcode sequences, (e.g., a barcode sequence that corresponds to the analyte binding moiety and/or the analyte).

A. Labeling Agent

In some embodiments, the labeling agent comprises an analyte-binding moiety (for example, an antibody) and a reporter oligonucleotide. In some embodiments, the labeling agent is a conjugate (e.g., an oligonucleotide-antibody conjugate).

(i) Analyte-Binding Moiety

In some embodiments, the analyte-binding moiety includes one or more antibodies or antigen binding fragments thereof. The antibodies or antigen binding fragments can specifically bind to a target analyte. In some embodiments, the analyte is a protein (e.g., a protein present on a surface of the biological sample (e.g., a cell) or an intracellular protein). In some embodiments, the antibody can be a polypeptide molecule that recognizes and binds to a complementary target antigen. Antibodies typically have a molecular structure shape that resembles a Y shape. Naturally-occurring antibodies, referred to as immunoglobulins, belong to one of the immunoglobulin classes IgG, IgM, IgA, IgD, and IgE. Antibodies can also be produced synthetically. In certain embodiments, the analyte-binding moiety can be a protein, a peptide, an antibody or an epitope binding fragment thereof, a lipophilic moiety, a cell surface receptor binding molecule, a receptor ligand, a small molecule, a bi-specific antibody, a pro-body, an aptamer, a monobody, an affimer, a darpin, and a protein scaffold, or any combination thereof. In some aspects, the analyte-binding moiety can be any moieties that can specifically target or bind to any analyte described herein, for example, in Section VI.B.

(ii) Reporter Oligonucleotide

In certain embodiments, the analyte-binding moiety is conjugated to a reporter oligonucleotide and a nucleic acid probe. In some aspects, the reporter oligonucleotide can comprise a sequence corresponding to the labeling agent and therefore corresponds to (e.g., uniquely identifies) the analyte. In some embodiments, the reporter oligonucleotide comprises one or more barcode sequences. In some embodiments, the reporter oligonucleotide comprises sequences that are complementary to, or partially complementary to, sequences present in the nucleic acid probe. In some embodiments, the nucleic acid probe can hybridize to the reporter oligonucleotide. In some embodiments, the hybridization of the reporter oligonucleotide and the nucleic acid probe permits ligation (e.g., circularization) of the nucleic acid probe, for example, via template ligation. In some embodiments, the nucleic acid probe can hybridized to the reporter oligonucleotide and ligated to generate a product for analysis.

B. Nucleic Acid Probe

FIG. 1 provides an exemplary schematic of a nucleic acid probe. In certain embodiments, the nucleic acid probe can be a circularizable probe (e.g., a padlock probe). In some embodiments, the nucleic acid probe (e.g., a padlock probe) contain one or more barcodes. The padlock probe described by the methods can comprise a nucleic acid sequence (e.g., a cassette) comprising from the 3′ to the 5′ end, an optional adapter sequence, a barcode, a nucleic acid capture sequence, and a restriction cleavage site. The cassette is a portion of the nucleic acid probe comprising a restriction site, a nucleic acid capture sequence, and a barcode sequence. The sequences can be directly adjacent to each other or could have intervening sequences (e.g., separated by one or more nucleotides). The rolling circle amplification product can comprise from the 5′ to the 3′ end, an optional adapter sequence, a barcode, a nucleic acid capture sequence, and a restriction cleavage site.

In some aspects, the adapter of the probe sequence can be a sequencing adapter sequence. In some aspects, the adapter can be used to prepare and sequence libraries of complementary DNA (cDNA) downstream.

In some embodiments, the nucleic acid probe can comprise a barcode sequence. In certain embodiments, the nucleic acid probe can contain an analyte-binding moiety-specific barcode (e.g., a protein barcode) and a spatial barcode. In a specific embodiment, the spatial barcode is optional. In certain embodiments, the analyte-binding moiety-specific barcode can be read in situ and be used to identify the specific analyte-binding moiety (e.g., a particular antibody) to which the reporter oligonucleotide is bound to, and the nucleic acid probe is hybridized to the reporter oligonucleotide.

Various nucleic acid probes can be hybridized to the labeling agent (e.g., via hybridizing to the reporter oligonucleotide) and each probe may comprise one or more barcode sequences. Exemplary barcoded nucleic acid probes or probe sets may be based on a padlock probe, a gapped padlock probe, a SNAIL (Splint Nucleotide Assisted Intramolecular Ligation) probe set, a PLAYR (Proximity Ligation Assay for RNA) probe set, a PLISH (Proximity Ligation in situ hybridization) probe set, and RNA-templated ligation probes. The specific nucleic acid probe or probe set design can vary.

In some embodiments, the optional spatial barcode comprises a contiguous nucleic acid segment or two or more non-contiguous nucleic acid segments that function as a label or identifier that conveys or is capable of conveying spatial information. In some embodiments, the nucleic acid probe includes a spatial barcode that possesses a spatial aspect, where the spatial barcode is associated with a particular location within the biological sample. In some aspects, the spatial barcode can be used to identify a particular molecule of analyte-binding moiety present in the biological sample.

The nucleic acid probe on each antibody can comprise a nucleic acid capture sequence. In certain embodiments, the nucleic acid capture sequence can be an oligo dA or an oligo dT. In certain embodiments, an amplification product (e.g., concatemer) of the nucleic acid probe, and/or the cleaved amplification products (e.g., monomer) contain sequences that are complementary to or nearly complementary to, the nucleic acid capture sequence. In some embodiments, the complements of the nucleic acid capture sequence can be an oligo dT or an oligo dA. In certain embodiments, after cleavage diffusion of the monomers, an oligo dT capture sequence present in the monomers binds to the polyA tail of a nearby mRNA or a second-strand cDNA. Alternatively, if the reverse transcription step has already been performed, the monomers produced by the cleavage of the concatemer will comprise an oligo dA capture sequence on the 3′ end. These monomers can diffuse into the surrounding tissue and target the oligo dT sequence of the circularized cDNA.

In some embodiments, the nucleic acid probe comprises a cassette comprising a restriction site or cleavage site. The cassette is a portion of the nucleic acid probe comprising a restriction site, a nucleic acid capture sequence, and a barcode sequence. The sequences can be directly adjacent to each other or could have intervening sequences (e.g., separated by one or more nucleotides). In some embodiments, the nucleic acid probe comprises a restriction site or cleavage site. After amplification to produce the concatemers, restriction endonucleases (e.g. DraI) can cleave at the restriction site which will leave the oligo dT at the 3′ end of the cleaved monomers. Examples of enzymes which will result in a terminal T at the end of the nucleic acid sequence include, but are not limited to, DraI (restriction site TTT/AAA), MseI (restriction site T/TAA), TaqI-v2 (restriction site T/CGA) and XbaI (restriction site T/CTAGA). In certain embodiments, endonucleases can cleave at the restriction site which will leave the oligo dA at the 3′end of the cleaved monomers. Examples of endonucleases which will result in a terminal A at the end of the nucleic acid sequence include, but are not limited to, AcII (restriction site AA/CGTT), AgeI (restriction site A/CCGGT), BgIII (restriction site A/GATCT), HindIII (restriction site A/AGCTT), SpeI (restriction site A/CTAGT), MluI (restriction site A/CGT), and PciI (restriction site A/CATGT). Following cleavage, the monomers will diffuse into the tissue and target a nearby nucleic acid sequence whereupon hybridization can occur.

### III. Nucleic Acid Probe Amplification

In some embodiments, provided herein are methods for analyzing a biological sample, such as for in situ analysis of one or more target nucleic acid(s) and/or one or more analyte(s), in a cell in a biological sample, such as an intact tissue sample, that involves the use of a labeling agent and a nucleic acid probe. In some aspects, the provided methods involve the generation of a concatemer of monomer sequences by amplification of the nucleic acid probe. In some embodiments, a nucleic acid probe (e.g., circularizable probe such as a padlock probe) is amplified through amplification methods such as rolling circle amplification.

A. Hybridization

In some embodiments, the labeling agent comprises a reporter oligonucleotide, which can be hybridized to a portion of the nucleic acid probe, thereby generating a hybridization product comprising the pairing of substantially complementary or complementary nucleic acid sequences within two different molecules, one of which is the reporter oligonucleotide conjugated to the analyte-binding moiety (e.g., antibody). The other molecule can be a nucleic acid probe. Pairing can be achieved by any process in which a nucleic acid sequence joins with a substantially or fully complementary sequence through base pairing to form a hybridization complex. For purposes of hybridization, two nucleic acid sequences are “substantially complementary” if at least 60% (e.g., at least 70%, at least 80%, or at least 90%) of their individual bases are complementary to one another.

B. Ligation

In some embodiments, the nucleic acid probe can be ligated prior to amplification. In some embodiments, the nucleic acid probe is ligated via an intramolecular ligation of a nucleic acid probe. In some embodiments, a ligation product is an intramolecular ligation of a nucleic acid probe, for example, the circularization of a circularizable nucleic acid probe or probe set, upon hybridization to the reporter oligonucleotide or a portion thereof.

In some embodiments, provided herein is a nucleic acid probe or probe set capable of DNA-templated ligation, such as from reporter oligonucleotide or a cDNA molecule. See, e.g., U.S. Pat. No. 8,551,710, which is hereby incorporated by reference in its entirety. In some embodiments, provided herein is a nucleic acid probe or probe set capable of RNA-templated ligation. See, e.g., U.S. Pat. Pub. 2020/0224244 which is hereby incorporated by reference in its entirety. In some embodiments, the probe set is a SNAIL probe set. See, e.g., U.S. Pat. Pub. 20190055594, which is hereby incorporated by reference in its entirety. In some embodiments, provided herein is a multiplexed proximity ligation assay. See, e.g., U.S. Pat. Pub. 20140194311 which is hereby incorporated by reference in its entirety. In some embodiments, provided herein is a nucleic acid probe or probe set capable of proximity ligation, for instance a proximity ligation assay for RNA (e.g., PLAYR) probe set. See, e.g., U.S. Pat. Pub. 20160108458, which is hereby incorporated by reference in its entirety. In some embodiments, a circular nucleic acid probe can be indirectly hybridized to the reporter oligonucleotide. In some embodiments, the circular construct is formed from a probe set capable of proximity ligation, for instance a proximity ligation in situ hybridization (PUSH) probe set. See, e.g., U.S. Pat. Pub. 2020/0224243 which is hereby incorporated by reference in its entirety.

In some embodiments, the ligation involves chemical ligation. In some embodiments, the ligation involves template dependent ligation. In some embodiments, the ligation involves template independent ligation. In some embodiments, the ligation involves enzymatic ligation.

In some embodiments, the enzymatic ligation involves use of a ligase. In some aspects, the ligase used herein comprises an enzyme that is commonly used to join polynucleotides together or to join the ends of a single polynucleotide. An RNA ligase, a DNA ligase, or another variety of ligase can be used to ligate two nucleotide sequences together. Ligases comprise ATP-dependent double-strand polynucleotide ligases, NAD-i-dependent double-strand DNA or RNA ligases and single-strand polynucleotide ligases, for example any of the ligases described in EC 6.5.1.1 (ATP-dependent ligases), EC 6.5.1.2 (NAD+-dependent ligases), EC 6.5.1.3 (RNA ligases). Specific examples of ligases comprise bacterial ligases such as E. coli DNA ligase, Tth DNA ligase, Thermococcus sp. (strain 9° N) DNA ligase (9° N™ DNA ligase, New England Biolabs), Taq DNA ligase, Ampligase™ (Epicentre Biotechnologies) and phage ligases such as T3 DNA ligase, T4 DNA ligase and T7 DNA ligase and mutants thereof. In some embodiments, the ligase is a T4 RNA ligase. In some embodiments, the ligase is a splintR ligase. In some embodiments, the ligase is a single stranded DNA ligase. In some embodiments, the ligase is a T4 DNA ligase. In some embodiments, the ligase is a ligase that has an DNA-splinted DNA ligase activity. In some embodiments, the ligase is a ligase that has an RNA-splinted DNA ligase activity.

In some embodiments, the ligation herein is a direct ligation. In some embodiments, the ligation herein is an indirect ligation. “Direct ligation” means that the ends of the polynucleotides hybridize immediately adjacently to one another to form a substrate for a ligase enzyme resulting in their ligation to each other (intramolecular ligation). Alternatively, “indirect” means that the ends of the polynucleotides hybridize non-adjacently to one another, e.g., separated by one or more intervening nucleotides or “gaps”. In some embodiments, said ends are not ligated directly to each other, but instead occurs either via the intermediacy of one or more intervening (so-called “gap” or “gap-filling” (oligo)nucleotides) or by the extension of the 3′ end of a probe to “fill” the “gap” corresponding to said intervening nucleotides (intermolecular ligation). In some cases, the gap of one or more nucleotides between the hybridized ends of the polynucleotides may be “filled” by one or more “gap” (oligo)nucleotide(s) which are complementary to a splint, nucleic acid probe (e.g., circularizable probe such as a padlock probe), or reporter oligonucleotide. The gap may be a gap of 1 to 60 nucleotides or a gap of 1 to 40 nucleotides or a gap of 3 to 40 nucleotides. In specific embodiments, the gap may be a gap of about 1, 2, 3, 4, 5, 6, 7, 8, 9 or 10 or more nucleotides, of any integer (or range of integers) of nucleotides in between the indicated values. In some embodiments, the gap between said terminal regions may be filled by a gap oligonucleotide or by extending the 3′ end of a polynucleotide. In some cases, ligation involves ligating the ends of the probe to at least one gap (oligo)nucleotide, such that the gap (oligo)nucleotide becomes incorporated into the resulting polynucleotide. In some embodiments, the ligation herein is preceded by gap filling. In other embodiments, the ligation herein does not require gap filling.

In some embodiments, ligation of the nucleic acid probe produces polynucleotides with melting temperature higher than that of unligated nucleic acid probe. Thus, in some aspects, ligation stabilizes the hybridization complex containing the ligated polynucleotides prior to subsequent steps, comprising amplification and detection.

In some aspects, a high fidelity ligase, such as a thermostable DNA ligase (e.g., a Taq DNA ligase), is used. Thermostable DNA ligases are active at elevated temperatures, allowing further discrimination by incubating the ligation at a temperature near the melting temperature (Tₘ) of the DNA strands. This selectively reduces the concentration of annealed mismatched substrates (expected to have a slightly lower Tₘ around the mismatch) over annealed fully base-paired substrates. Thus, high-fidelity ligation can be achieved through a combination of the intrinsic selectivity of the ligase active site and balanced conditions to reduce the incidence of annealed mismatched dsDNA.

In some embodiments, the ligation herein is a proximity ligation of ligating two (or more) nucleic acid sequences that are in proximity with each other, e.g., through enzymatic means (e.g., a ligase). In some embodiments, proximity ligation can include a “gap-filling” step that involves incorporation of one or more nucleic acids by a polymerase, based on the nucleic acid sequence of a template nucleic acid molecule, spanning a distance between the two nucleic acid molecules of interest (see, e.g., U.S. Pat. No. 7,264,929, the entire contents of which are incorporated herein by reference). A wide variety of different methods can be used for proximity ligating nucleic acid molecules, including (but not limited to) “sticky-end” and “blunt-end” ligations. Additionally, single-stranded ligation can be used to perform proximity ligation on a single-stranded nucleic acid molecule. Sticky-end proximity ligations involve the hybridization of complementary single-stranded sequences between the two nucleic acid molecules to be joined, prior to the ligation event itself. Blunt-end proximity ligations generally do not include hybridization of complementary regions from each nucleic acid molecule because both nucleic acid molecules lack a single-stranded overhang at the site of ligation.

C. Amplification

In some embodiments, the nucleic acid probe, e.g., a circular probe or circularizable probe or probe set, is amplified. In some embodiments, the amplifying is achieved by performing rolling circle amplification (RCA). In some embodiments, a primer that hybridizes to the nucleic acid probe, e.g., a circular probe or circularized probe is present and used to initiate amplification. In some embodiments, the primer for amplification is comprised in the reporter oligonucleotide that is conjugated to the analyte-binding moiety. In some aspects, hybridization of the reporter oligonucleotide and the nucleic acid probe can initiate amplification, e.g., by RCA. In some embodiments, the RCA comprises a linear RCA, a branched RCA, a dendritic RCA, or any combination thereof.

In some embodiments, the amplification is performed at a temperature between or between about 20° C. and about 60° C. In some embodiments, the amplification is performed at a temperature between or between about 30° C. and about 40° C. In some aspects, the amplification step, such as the rolling circle amplification (RCA) is performed at a temperature between at or about 25° C. and at or about 50° C., such as at or about 25° C., 27° C., 29° C., 31° C., 33° C., 35° C., 37° C., 39° C., 41° C., 43° C., 45° C., 47° C., or 49° C.

In some embodiments, upon addition of a DNA polymerase in the presence of appropriate dNTP precursors and other cofactors, a primer is elongated to produce multiple copies of the circular template. This amplification step can utilize isothermal amplification or non-isothermal amplification. In some embodiments, after the formation of the hybridization complex and association of the amplification probe, the hybridization complex is rolling-circle amplified to generate a cDNA nanoball (e.g., amplicon) containing multiple copies of the cDNA. Any suitable techniques for rolling circle amplification (RCA) such as linear RCA, a branched RCA, a dendritic RCA, or any combination thereof can be used. (See, e.g., Baner et al, Nucleic Acids Research, 26:5073-5078, 1998; Lizardi et al, Nature Genetics 19:226, 1998; Mohsen et al., Acc Chem Res. 2016 Nov. 15; 49(11): 2540-2550; Schweitzer et al. Proc. Natl Acad. Sci. USA 97:101 13-1 19, 2000; Faruqi et al, BMC Genomics 2:4, 2000; Nallur et al, Nucl. Acids Res. 29:el 18, 2001; Dean et al. Genome Res. 11:1095-1099, 2001; Schweitzer et al, Nature Biotech. 20:359-365, 2002; U.S. Pat. Nos. 6,054,274, 6,291,187, 6,323,009, 6,344,329 and 6,368,801). Exemplary polymerases for use in RCA comprise DNA polymerase such phi29 (φ29) polymerase, Klenow fragment, Bacillus stearothermophilus DNA polymerase (BST), T4 DNA polymerase, T7 DNA polymerase, or DNA polymerase I. In some aspects, DNA polymerases that have been engineered or mutated to have desirable characteristics can be employed. In some embodiments, the polymerase is phi29 DNA polymerase.

In some aspects, during the amplification step, modified nucleotides can be added to the reaction to incorporate the modified nucleotides in the amplification product (e.g., nanoball). Exemplary of the modified nucleotides comprise amine-modified nucleotides. In some aspects of the methods, for example, for anchoring or cross-linking of the generated amplification product (e.g., nanoball) to a scaffold, to cellular structures and/or to other amplification products (e.g., other nanoballs). In some aspects, the amplification products comprises a modified nucleotide, such as an amine-modified nucleotide. In some embodiments, the amine-modified nucleotide comprises an acrylic acid N-hydroxysuccinimide moiety modification. Examples of other amine-modified nucleotides comprise, but are not limited to, a 5-Aminoallyl-dUTP moiety modification, a 5-Propargylamino-dCTP moiety modification, a N6-6-Aminohexyl-dATP moiety modification, or a 7-Deaza-7-Propargylamino-dATP moiety modification.

In some aspects, the polynucleotides and/or amplification product (e.g., amplicon) can be anchored to a polymer matrix. For example, the polymer matrix can be a hydrogel. In some embodiments, one or more of the polynucleotide probe(s) can be modified to contain functional groups that can be used as an anchoring site to attach the polynucleotide probes and/or amplification product to a polymer matrix. Exemplary modification and polymer matrix that can be employed in accordance with the provided embodiments comprise those described in, for example, WO 2014/163886, WO 2017/079406, US 2016/0024555, US 2018/0251833 and US 2017/0219465. In some examples, the scaffold also contains modifications or functional groups that can react with or incorporate the modifications or functional groups of the probe set or amplification product. In some examples, the scaffold can comprise oligonucleotides, polymers or chemical groups, to provide a matrix and/or support structures.

The amplification products may be immobilized within the matrix generally at the location of the nucleic acid being amplified, thereby creating a localized colony of amplicons. The amplification products may be immobilized within the matrix by steric factors. The amplification products may also be immobilized within the matrix by covalent or noncovalent bonding. In this manner, the amplification products may be considered to be attached to the matrix. By being immobilized to the matrix, such as by covalent bonding or cross-linking, the size and spatial relationship of the original amplicons is maintained. By being immobilized to the matrix, such as by covalent bonding or cross-linking, the amplification products are resistant to movement or unraveling under mechanical stress.

In some aspects, the amplification products are copolymerized and/or covalently attached to the surrounding matrix thereby preserving their spatial relationship and any information inherent thereto. For example, if the amplification products are those generated from DNA or RNA within a cell embedded in the matrix, the amplification products can also be functionalized to form covalent attachment to the matrix preserving their spatial information within the cell thereby providing a subcellular localization distribution pattern. In some embodiments, the provided methods involve embedding the one or more polynucleotide probe sets and/or the amplification products in the presence of hydrogel subunits to form one or more hydrogel-embedded amplification products. In some embodiments, the hydrogel-tissue chemistry described comprises covalently attaching nucleic acids to in situ synthesized hydrogel for tissue clearing, enzyme diffusion, and multiple-cycle sequencing while an existing hydrogel-tissue chemistry method cannot. In some embodiments, to enable amplification product embedding in the tissue-hydrogel setting, amine-modified nucleotides are comprised in the amplification step (e.g., RCA), functionalized with an acrylamide moiety using acrylic acid N-hydroxysuccinimide esters, and copolymerized with acrylamide monomers to form a hydrogel.

In some embodiments, the RCA template may comprise the nucleic acid probe, or a part thereof, where the nucleic acid probe comprises a nucleic acid, and it may be provided or generated as a proxy, or a marker, for the analyte (e.g., associated with a particular labeling agent or analyte-binding moiety). As noted above, many assays are known for the detection of numerous different analytes, which use a RCA-based detection system, e.g., where the signal is provided by generating a RCP from a circular RCA template which is provided or generated in the assay, and the RCP is detected to detect the analyte. The RCP may thus be regarded as a reporter which is detected to detect the analyte. In some embodiments, RCP is generated based on the RCA template, and comprises complementary copies of the RCA template (e.g., nucleic acid probe). The RCA template determines the signal which is detected, and is thus indicative of the nucleic acid probe and the analyte. As will be described in more detail below, the RCA template may be a nucleic acid probe, or a part or component of a nucleic acid probe, or may be generated from a nucleic acid probe, or it may be a component of a detection assay (e.g., a reagent in a detection assay), which is used as a reporter for the assay, or a part of a reporter, or signal-generation system. The RCA template used to generate the RCP may thus be a circular (e.g. circularized) nucleic acid probe, namely from any RCA-based detection assay which uses or generates a circular nucleic acid probe as a reporter for the assay. Since the RCA template generates the RCP reporter, it may be viewed as part of the reporter system for the assay.

In some embodiments, a product herein includes a molecule or a complex generated in a series of reactions, e.g., hybridization, ligation, extension, replication, transcription/reverse transcription, and/or amplification (e.g., rolling circle amplification), in any suitable combination. For example, a product comprising a target sequence for a probe disclosed herein (e.g., a detectably labelled probe comprising a quencher moiety) may be a hybridization complex formed of a cellular nucleic acid in a sample and an exogenously added nucleic acid probe. The exogenously added nucleic acid probe may comprise an overhang that does not hybridize to the cellular nucleic acid but hybridizes to another probe (e.g., a detectably labelled comprising a quencher moiety). The exogenously added nucleic acid probe may be optionally ligated to a cellular nucleic acid molecule or another exogenous nucleic acid molecule. In other examples, a product comprising a target sequence for a probe disclosed herein (e.g., a detectably labelled probe comprising a quencher moiety) may be an RCP of a circularizable nucleic acid probe or probe set which hybridizes to a cellular nucleic acid molecule (e.g., genomic DNA or mRNA) or product thereof (e.g., a transcript such as cDNA, a DNA-templated ligation product of two probes, or an RNA-templated ligation product of two probes). In other examples, a product comprising a target sequence for a probe disclosed herein (e.g., a detectably labelled probe comprising a quencher moiety) may a probe hybridizing to an RCP. The probe may comprise an overhang that does not hybridize to the RCP but hybridizes to another probe (e.g., a detectably labelled probe comprising a quencher moiety). The probe may be optionally ligated to a cellular nucleic acid molecule or another probe, e.g., an anchor probe that hybridize to the RCP.

### IV. Generation of Monomers and Target Nucleic Acid Capture

In some embodiments, the provided methods involve the generation of a plurality of monomers comprising sequences that are complementary to the nucleic acid probes, by cleaving the concatemer (e.g., cleaving amplification product of the nucleic acid probe via restriction endonuclease digestion). In some aspects, the generated monomers contain sequences that are complementary or nearly complementary (e.g., a complement), to the nucleic acid capture sequence present in the nucleic acid probe. In some aspects, the complement of the nucleic acid capture sequence, present in the monomers, can diffuse in the biological sample to bind and/or capture target nucleic acid (e.g., mRNA or cDNA of the mRNA) present in the sample. In some aspects, diffusion allows the monomers to bind and/or capture target nucleic acids that are physically located nearby or proximate to the monomers. In some aspects, by virtue of the plurality monomers being generated in situ near the location of the labeling agent and nucleic acid probe, the methods provide an ability to bind to, capture and/or tag various target nucleic acids that are physically located nearby the analyte, in a sequence non-specific manner or a sequence-specific manner. In some aspects, the provided embodiments can be employed for investigating protein-RNA interaction or the distribution of RNA at particular locations.

A. Digestion and Hybridization

In some embodiments, the provided methods involve the generation of a plurality of monomers by cleaving the concatemer (e.g., via restriction endonuclease digestion). In certain embodiments, the nucleic acid probe on each analyte-binding moiety can comprise a restriction site or cleavage site.

After amplification to produce the concatemers, enzymes (e.g. DraI) can cleave at the restriction site which will leave the oligo dT at the 3′ end cleavage. Examples of enzymes which will result in a terminal T at the end of the nucleic acid sequence include, but are not limited to, DraI (restriction site TTT/AAA), MseI (restriction site T/TAA), TaqI-v2 (restriction site T/CGA) and XbaI (restriction site T/CTAGA). In certain embodiments, endonucleases can cleave at the restriction site which will leave the oligo dA at the 3′ end cleavage. Examples of endonucleases which will result in a terminal A at the end of the nucleic acid sequence include, but are not limited to, AcII (restriction site AA/CGTT), AgeI (restriction site A/CCGGT), BgIII (restriction site A/GATCT), HindIII (restriction site A/AGCTT), SpeI (restriction site A/CTAGT), MluI (restriction site A/CGT), and PciI (restriction site A/CATGT).

In some embodiments, the monomers can diffuse into the surrounding area of the biological sample, in which the complement of the nucleic acid capture sequence, at the 3′ end of the monomer hybridizes to and captures the target nucleic acid (e.g., polyA tail of the mRNA). In alternate embodiments, monomers that comprise a oligo dA capture sequence on the 3′ end can diffuse into the biological sample and hybridize to the oligo dT sequence of the circularized cDNA.

B. Capture and Priming of mRNA Molecules

In some embodiments, the target nucleic acid is an mRNA present in the biological sample. In some embodiments, the cleaved monomers can bind to and capture mRNA molecules present near the monomers (and by extension, labeling agents and analytes targeted by the analyte-binding moiety of the labeling agent). In some aspects, the complement of the nucleic acid capture sequence at the 3′ end of the monomer, e.g., comprising a plurality of thymine (T) sequences, hybridizes to and captures the target nucleic acid (e.g., polyA tail of the mRNA).

FIG. 2 depicts a schematic of an exemplary method of an in situ assay. In some embodiments, the methods involve the targeting of an analyte (e.g., protein) using an analyte-binding agent (e.g., an antibody) conjugated with a reporter oligonucleotide, and a nucleic acid probe (e.g., circularizable probe such as a padlock probe) that can be hybridized to it. The padlock probe comprises specific sequences as described above. In certain embodiments, the antibody can bind to an analyte on the biological sample (e.g., tissue section), whereupon rolling circle amplification of the nucleic acid probe is performed.

The amplification of the probe can generate a concatemer of monomer sequences, wherein each monomer is complementary to the sequence of the probe. In some aspects, the concatemer can be detected at a spatial location of the biological sample by in situ sequencing and/or in situ hybridization. The concatemer can be transformed into individual monomers upon cleavage at the restriction sites. Enzymes that can cleave the concatemer at the restriction sites include, but are not limited to, DraI, MseI, TaqI-v2 and XbaI. The monomers produced by the cleavage of the concatemer comprises a plurality of thymine (T) (e.g., oligo dT) as nucleic acid capture sequence on the 3′ end. Upon diffusing into the surrounding area in the biological sample, the complement (e.g., oligo dT) of the monomer can target the polyA tail of nearby mRNAs.

In some embodiments, once captured, the monomer can hybridize to the mRNA and reverse transcription (RT) can occur by using the complement in the monomers as a reverse transcription primer. In some embodiments, the generated library of tagged cDNA can be altered by ligating or adding a second adapter sequence to the tagged cDNA. Additionally, the tagged cDNA can be amplified prior to being analyzed by direct sequencing or indirect sequencing. The amplified sequence can include the protein barcode sequence and/or the spatial barcode sequence. In certain embodiments, the protein barcode can be read in situ and be used to identify the specific antibody to which the oligonucleotide and padlock probe was bound to. The optional spatial barcode can be used to define the position of the single antibody in situ.

In some embodiments, reverse transcription of the mRNA is performed in the presence of a modified nucleotide. In some aspects, the modified nucleotide comprises a cross linkable nucleotide. In some aspects, the modified nucleotide is a cross linkable nucleotide. Exemplary modified nucleotides comprise halogenated base, an azide-modified base, an aminoallyl dUTP, an octadiynyl dU, a thiol-modified base, a biotin-modified base, or any combination thereof. In some embodiments, the method further comprises crosslinking the modified nucleotide to the sample, a substrate, and/or a matrix, thereby crosslinking the cDNA to the sample, the substrate, and/or the matrix.

C. Capture and Priming of cDNA Molecules

In certain embodiments, in situ reverse transcription of the mRNA can occur before amplification of the nucleic acid probe. In some embodiments, the target nucleic acid comprises a reverse transcription product of an mRNA molecule, optionally a complementary DNA (cDNA). In some aspects, the target nucleic acid is complementary DNA (cDNA) of mRNAs present in the biological sample.

FIG. 3 depicts a schematic of an exemplary method of an in situ assay. mRNAs present in the biological sample is reverse transcribed to generate cDNA molecules which are then circularized, for example, by way of a single-stranded DNA ligase that catalyzes intramolecular ligation. As shown in FIG. 3, an analyte-binding agent (e.g., antibody) binds to an analyte, and the analyte-binding agent can be conjugated with a reporter oligonucleotide, and a nucleic acid probe (e.g., a padlock probe) can be hybridized to it. In some aspects, the nucleic acid probe is amplified by RCA to generate a concatemer of monomers. In some aspects, the concatemer can be detected at a spatial location of the biological sample by in situ sequencing and/or in situ hybridization. The concatemer can be cleaved into monomers at the restriction cleavage site, for example, using restriction endonucleases. The endonucleases responsible for cleavage can be, but are not limited to AcII, AgeI, BgIII, HindIII, SpeI, MluI, and PciI. The monomers produced by the cleavage of the concatemer can comprise a plurality of adenine (A) nucleotides (e.g., oligo dA) as the complement of the nucleic acid capture sequence on the 3′ end. Upon diffusing into the surrounding area in the biological sample, the complement (e.g., oligo dA) of the monomer can target the oligo dT sequence present in the nearby circularized cDNA. In some aspects, a library of tagged cDNA can be generated by priming the extension of the circularized cDNA with the complement in the monomer as an extension primer.

In some embodiments, the target nucleic acid is a second-strand cDNA of an mRNA molecule in the biological sample. In some aspects, the second-strand cDNA is generated by reverse transcription of the mRNA molecules in the biological sample and generating the second-strand cDNA using a template-switch oligonucleotide (TSO), or other methods of generating a second-strand cDNA. In some aspects, the second-strand cDNA comprises a plurality of adenine (A) nucleotides at the 3′ end of the cDNA, which corresponds to the polyA tail of the mRNA.

In some embodiments, the methods involve the targeting of an analyte (e.g., protein) using an analyte-binding agent (e.g., an antibody) conjugated with a reporter oligonucleotide, and a nucleic acid probe (e.g., circularizable probe such as a padlock probe) that can be hybridized to it. The padlock probe comprises specific sequences as described above. In certain embodiments, the antibody can bind to an analyte on the biological sample (e.g., tissue section), whereupon rolling circle amplification of the nucleic acid probe is performed.

The amplification of the probe can generate a concatemer of monomer sequences, wherein each monomer is complementary to the sequence of the probe. In some aspects, the concatemer can be detected at a spatial location of the biological sample by in situ sequencing and/or in situ hybridization. The concatemer can be transformed into individual monomers upon cleavage at the restriction sites. Enzymes that can cleave the concatemer at the restriction sites include, but are not limited to, DraI, MseI, TaqI-v2 and XbaI. The monomers produced by the cleavage of the concatemer comprises a plurality of thymine (T) (e.g., oligo dT) as nucleic acid capture sequence on the 3′ end. Upon diffusing into the surrounding area in the biological sample, the complement (e.g., oligo dT) of the monomer can target the oligo dA sequences that are present in the nearby second-strand cDNAs (e.g., corresponding to the polyA tail of mRNAs).

### V. Library Preparation, Generation, and Sequencing

In some embodiments, the methods described herein also include generating a library of tagged complementary DNA (cDNA). In some embodiments, the library is generated by performing reverse transcription (RT) of the target nucleic acids (e.g., mRNA present in the biological sample and hybridized to a portion of the monomers) using the complement in the monomer as a reverse transcription primer. In some embodiments, the library is generated by performing primer extension of the target nucleic acids (e.g., cDNA that is a reverse transcription product of mRNA present in the biological sample, and hybridized to a portion of the monomers) using the complement in the monomer as an extension primer.

A primer is generally a single-stranded nucleic acid sequence having a 3′ end that can be used as a substrate for a nucleic acid polymerase in a nucleic acid extension reaction. RNA primers are formed of RNA nucleotides, and are used in RNA synthesis, while DNA primers are formed of DNA nucleotides and used in DNA synthesis. Primers can also include both RNA nucleotides and DNA nucleotides (e.g., in a random or designed pattern). Primers can also include other natural or synthetic nucleotides described herein that can have additional functionality. In some examples, DNA primers can be used to prime RNA synthesis and vice versa (e.g., RNA primers can be used to prime DNA synthesis). Primers can vary in length. For example, primers can be about 6 bases to about 120 bases. For example, primers can include up to about 25 bases. A primer, may in some cases, refer to a primer binding sequence. A primer extension reaction generally refers to any method where two nucleic acid sequences become linked (e.g., hybridized) by an overlap of their respective terminal complementary nucleic acid sequences (e.g., 3′ termini). Such linking can be followed by nucleic acid extension (e.g., an enzymatic extension) of one, or both termini using the other nucleic acid sequence as a template for extension. Enzymatic extension can be performed by an enzyme including, but not limited to, a polymerase and/or a reverse transcriptase.

Exemplary steps for DNA generation (e.g., first strand cDNA generation and second strand generation), DNA amplification (e.g., cDNA amplification) and quality control, and spatial gene expression library construction are disclosed for example in WO 2020/047002, WO 2020/047004, US 2021/0332424, WO 2020/047005, US 2021/0317524, WO 2020/047007, US 2021/0324457, WO 2020/047010, and US 2021/0332425 all of which are incorporated herein by reference in their entireties.

In some embodiments, a tagged cDNA library is constructed and analyzed. In some embodiments, target nucleic acids are pre-processed for library generation via next generation sequencing. For example, target nucleic acids can be pre-processed by addition of a modification (e.g., ligation of sequences that allow interaction with the monomers). In some embodiments, target nucleic acids (e.g., DNA or RNA) are fragmented using fragmentation techniques (e.g., using transposases and/or fragmentation buffers).

In some aspects, the tagged cDNA library or a portion of the library can be detected or sequenced in situ, and/or via high throughput sequencing methods. In some aspects, the library of tagged cDNA or a portion thereof can be further assessed by in situ detection, imaging and/or sequencing, or using high-throughput sequencing methods, for example, as described herein and/or using standard high-throughput sequencing chemistries and deep three dimensional imaging for high throughput information readout. In some aspects, one or more barcodes that are present in the monomer can be used to identify the analyte (e.g., via specifying particular analyte-binding moieties) and/or and a particular spatial location (e.g., spatial barcode can specify particular location of the biological sample). In situ detection, analysis and/or sequencing of the tagged cDNA library or a portion thereof, can be performed using any such methods, including methods described herein, for example, in Section VII.

In some embodiments, the library of tagged cDNA can also be analyzed by sequencing. The generated library of tagged cDNA can be altered by ligating or adding a second adapter sequence to the tagged cDNA. Additionally, the tagged cDNA can be amplified prior to being analyzed by direct sequencing or indirect sequencing. The amplified sequence can include one or more barcode sequences, such as the analyte-binding moiety specific barcode and/or the spatial barcode sequence. In certain embodiments, the analyte-binding moiety specific barcode can be read in situ and be used to identify the specific analyte-binding moiety (e.g., antibody) to which the reporter oligonucleotide and the nucleic acid probe was bound to. The optional spatial barcode can be used to define the position of the single analyte-binding moiety in situ.

Exemplary high-throughput sequencing chemistries that utilize fluorescence imaging comprise ABI SoLiD (Life Technologies), in which a sequencing primer on a template is ligated to a library of fluorescently labeled nonamers with a cleavable terminator. After ligation, the beads are then imaged using four color channels (FITC, Cy3, Texas Red and Cy5). The terminator is then cleaved off leaving a free-end to engage in the next ligation-extension cycle. After all dinucleotide combinations have been determined, the images are mapped to the color code space to determine the specific base calls per template. The workflow is achieved using an automated fluidics and imaging device (e.g., SoLiD 5500 W Genome Analyzer, ABI Life Technologies). Another sequencing platform uses sequencing by synthesis, in which a pool of single nucleotide with a cleavable terminator is incorporated using DNA polymerase. After imaging, the terminator is cleaved and the cycle is repeated. The fluorescence images are then analyzed to call bases for each DNA amplicons within the flow cell (HiSeq, Illumina).

### VI. Samples, Analytes, and Target Sequences

A. Samples

A sample disclosed herein can be or derived from any biological sample. Methods and compositions disclosed herein may be used for analyzing a biological sample, which may be obtained from a subject using any of a variety of techniques including, but not limited to, biopsy, surgery, and laser capture microscopy (LCM), and generally includes cells and/or other biological material from the subject. In addition to the subjects described above, a biological sample can be obtained from a prokaryote such as a bacterium, an archaea, a virus, or a viroid. A biological sample can also be obtained from non-mammalian organisms (e.g., a plant, an insect, an arachnid, a nematode, a fungus, or an amphibian). A biological sample can also be obtained from a eukaryote, such as a tissue sample, a patient derived organoid (PDO) or patient derived xenograft (PDX). A biological sample from an organism may comprise one or more other organisms or components therefrom. For example, a mammalian tissue section may comprise a prion, a viroid, a virus, a bacterium, a fungus, or components from other organisms, in addition to mammalian cells and non-cellular tissue components. Subjects from which biological samples can be obtained can be healthy or asymptomatic individuals, individuals that have or are suspected of having a disease (e.g., a patient with a disease such as cancer) or a pre-disposition to a disease, and/or individuals in need of therapy or suspected of needing therapy.

The biological sample can include any number of macromolecules, for example, cellular macromolecules and organelles (e.g., mitochondria and nuclei). The biological sample can be a nucleic acid sample and/or protein sample. The biological sample can be a carbohydrate sample or a lipid sample. The biological sample can be obtained as a tissue sample, such as a tissue section, biopsy, a core biopsy, needle aspirate, or fine needle aspirate. The sample can be a fluid sample, such as a blood sample, urine sample, or saliva sample. The sample can be a skin sample, a colon sample, a cheek swab, a histology sample, a histopathology sample, a plasma or serum sample, a tumor sample, living cells, cultured cells, a clinical sample such as, for example, whole blood or blood-derived products, blood cells, or cultured tissues or cells, including cell suspensions. In some embodiments, the biological sample may comprise cells which are deposited on a surface.

Cell-free biological samples can include extracellular polynucleotides. Extracellular polynucleotides can be isolated from a bodily sample, e.g., blood, plasma, serum, urine, saliva, mucosal excretions, sputum, stool, and tears.

Biological samples can be derived from a homogeneous culture or population of the subjects or organisms mentioned herein or alternatively from a collection of several different organisms, for example, in a community or ecosystem.

Biological samples can include one or more diseased cells. A diseased cell can have altered metabolic properties, gene expression, protein expression, and/or morphologic features. Examples of diseases include inflammatory disorders, metabolic disorders, nervous system disorders, and cancer. Cancer cells can be derived from solid tumors, hematological malignancies, cell lines, or obtained as circulating tumor cells. Biological samples can also include fetal cells and immune cells.

Biological samples can include analytes (e.g., protein, RNA, and/or DNA) embedded in a 3D matrix. In some embodiments, amplicons (e.g., rolling circle amplification products) derived from or associated with analytes (e.g., protein, RNA, and/or DNA) can be embedded in a 3D matrix. In some embodiments, a 3D matrix may comprise a network of natural molecules and/or synthetic molecules that are chemically and/or enzymatically linked, e.g., by crosslinking. In some embodiments, a 3D matrix may comprise a synthetic polymer. In some embodiments, a 3D matrix comprises a hydrogel.

In some embodiments, a substrate herein can be any support that is insoluble in aqueous liquid and which allows for positioning of biological samples, analytes, features, and/or reagents (e.g., probes) on the support. In some embodiments, a biological sample can be attached to a substrate. Attachment of the biological sample can be irreversible or reversible, depending upon the nature of the sample and subsequent steps in the analytical method. In certain embodiments, the sample can be attached to the substrate reversibly by applying a suitable polymer coating to the substrate, and contacting the sample to the polymer coating. The sample can then be detached from the substrate, e.g., using an organic solvent that at least partially dissolves the polymer coating. Hydrogels are examples of polymers that are suitable for this purpose.

In some embodiments, the substrate can be coated or functionalized with one or more substances to facilitate attachment of the sample to the substrate. Suitable substances that can be used to coat or functionalize the substrate include, but are not limited to, lectins, poly-lysine, antibodies, and polysaccharides.

A variety of steps can be performed to prepare or process a biological sample for and/or during an assay. Except where indicated otherwise, the preparative or processing steps described below can generally be combined in any manner and in any order to appropriately prepare or process a particular sample for and/or analysis.

### (i) Tissue Sectioning

A biological sample can be harvested from a subject (e.g., via surgical biopsy, whole subject sectioning) or grown in vitro on a growth substrate or culture dish as a population of cells, and prepared for analysis as a tissue slice or tissue section. Grown samples may be sufficiently thin for analysis without further processing steps. Alternatively, grown samples, and samples obtained via biopsy or sectioning, can be prepared as thin tissue sections using a mechanical cutting apparatus such as a vibrating blade microtome. As another alternative, in some embodiments, a thin tissue section can be prepared by applying a touch imprint of a biological sample to a suitable substrate material.

The thickness of the tissue section can be a fraction of (e.g., less than 0.9, 0.8, 0.7, 0.6, 0.5, 0.4, 0.3, 0.2, or 0.1) the maximum cross-sectional dimension of a cell. However, tissue sections having a thickness that is larger than the maximum cross-section cell dimension can also be used. For example, cryostat sections can be used, which can be, e.g., 10-20 μm thick.

More generally, the thickness of a tissue section typically depends on the method used to prepare the section and the physical characteristics of the tissue, and therefore sections having a wide variety of different thicknesses can be prepared and used. For example, the thickness of the tissue section can be at least 0.1, 0.2, 0.3, 0.4, 0.5, 0.7, 1.0, 1.5, 2, 3, 4, 5, 6, 7, 8, 9, 10, 12, 13, 14, 15, 20, 30, 40, or 50 μm. Thicker sections can also be used if desired or convenient, e.g., at least 70, 80, 90, or 100 μm or more. Typically, the thickness of a tissue section is between 1-100 μm, 1-50 μm, 1-30 μm, 1-25 μm, 1-20 μm, 1-15 μm, 1-10 μm, 2-8 μm, 3-7 μm, or 4-6 μm, but as mentioned above, sections with thicknesses larger or smaller than these ranges can also be analysed.

Multiple sections can also be obtained from a single biological sample. For example, multiple tissue sections can be obtained from a surgical biopsy sample by performing serial sectioning of the biopsy sample using a sectioning blade. Spatial information among the serial sections can be preserved in this manner, and the sections can be analysed successively to obtain three-dimensional information about the biological sample.

### (ii) Freezing

In some embodiments, the biological sample (e.g., a tissue section as described above) can be prepared by deep freezing at a temperature suitable to maintain or preserve the integrity (e.g., the physical characteristics) of the tissue structure. The frozen tissue sample can be sectioned, e.g., thinly sliced, onto a substrate surface using any number of suitable methods. For example, a tissue sample can be prepared using a chilled microtome (e.g., a cryostat) set at a temperature suitable to maintain both the structural integrity of the tissue sample and the chemical properties of the nucleic acids in the sample. Such a temperature can be, e.g., less than −15° C., less than −20° C., or less than −25° C.

(iii) Fixation and Postfixation

In some embodiments, the biological sample can be prepared using formalin-fixation and paraffin-embedding (FFPE), which are established methods. In some embodiments, cell suspensions and other non-tissue samples can be prepared using formalin-fixation and paraffin-embedding. Following fixation of the sample and embedding in a paraffin or resin block, the sample can be sectioned as described above. Prior to analysis, the paraffin-embedding material can be removed from the tissue section (e.g., deparaffinization) by incubating the tissue section in an appropriate solvent (e.g., xylene) followed by a rinse (e.g., 99.5% ethanol for 2 minutes, 96% ethanol for 2 minutes, and 70% ethanol for 2 minutes).

As an alternative to formalin fixation described above, a biological sample can be fixed in any of a variety of other fixatives to preserve the biological structure of the sample prior to analysis. For example, a sample can be fixed via immersion in ethanol, methanol, acetone, paraformaldehyde (PFA)-Triton, and combinations thereof.

In some embodiments, acetone fixation is used with fresh frozen samples, which can include, but are not limited to, cortex tissue, mouse olfactory bulb, human brain tumor, human post-mortem brain, and breast cancer samples. When acetone fixation is performed, pre-permeabilization steps (described below) may not be performed. Alternatively, acetone fixation can be performed in conjunction with permeabilization steps.

In some embodiments, the methods provided herein comprises one or more post-fixing (also referred to as postfixation) steps. In some embodiments, one or more post-fixing step is performed after contacting a sample with a polynucleotide disclosed herein, e.g., one or more probes such as a circularizable probe (e.g., circular or padlock probe). In some embodiments, one or more post-fixing step is performed after a hybridization complex comprising a probe and a target is formed in a sample. In some embodiments, one or more post-fixing step is performed prior to a ligation reaction disclosed herein, such as the ligation to circularize a padlock probe.

In some embodiments, one or more post-fixing step is performed after contacting a sample with a binding or labeling agent (e.g., an antibody or antigen binding fragment thereof) for a non-nucleic acid analyte such as a protein analyte. The labeling agent can comprise a nucleic acid molecule (e.g., reporter oligonucleotide) comprising a sequence corresponding to the labeling agent and therefore corresponds to (e.g., uniquely identifies) the analyte. In some embodiments, the labeling agent can comprise a reporter oligonucleotide comprising one or more barcode sequences.

A post-fixing step may be performed using any suitable fixation reagent disclosed herein, for example, 3% (w/v) paraformaldehyde in DEPC-PBS.

### (iv) Embedding

As an alternative to paraffin embedding described above, a biological sample can be embedded in any of a variety of other embedding materials to provide structural substrate to the sample prior to sectioning and other handling steps. In some cases, the embedding material can be removed, e.g., prior to analysis of tissue sections obtained from the sample. Suitable embedding materials include, but are not limited to, waxes, resins (e.g., methacrylate resins), epoxies, and agar.

In some embodiments, the biological sample can be embedded in a matrix (e.g., a hydrogel matrix). Embedding the sample in this manner typically involves contacting the biological sample with a hydrogel such that the biological sample becomes surrounded by the hydrogel. For example, the sample can be embedded by contacting the sample with a suitable polymer material, and activating the polymer material to form a hydrogel. In some embodiments, the hydrogel is formed such that the hydrogel is internalized within the biological sample.

In some embodiments, the biological sample is immobilized in the hydrogel via cross-linking of the polymer material that forms the hydrogel. Cross-linking can be performed chemically and/or photochemically, or alternatively by any other suitable hydrogel-formation method.

The composition and application of the hydrogel-matrix to a biological sample typically depends on the nature and preparation of the biological sample (e.g., sectioned, non-sectioned, type of fixation). As one example, where the biological sample is a tissue section, the hydrogel-matrix can include a monomer solution and an ammonium persulfate (APS) initiator/tetramethylethylenediamine (TEMED) accelerator solution. As another example, where the biological sample consists of cells (e.g., cultured cells or cells disassociated from a tissue sample), the cells can be incubated with the monomer solution and APS/TEMED solutions. For cells, hydrogel-matrix gels are formed in compartments, including but not limited to devices used to culture, maintain, or transport the cells. For example, hydrogel-matrices can be formed with monomer solution plus APS/TEMED added to the compartment to a depth ranging from about 0.1 μm to about 2 mm.

Additional methods and aspects of hydrogel embedding of biological samples are described for example in Chen et al., Science 347(6221):543-548, 2015, the entire contents of which are incorporated herein by reference.

### (v) Staining

To facilitate visualization, biological samples can be stained using a wide variety of stains and staining techniques. In some embodiments, for example, a sample can be stained using any number of stains and/or immunohistochemical reagents. One or more staining steps may be performed to prepare or process a biological sample for an assay described herein or may be performed during and/or after an assay. In some embodiments, the sample can be contacted with one or more nucleic acid stains, membrane stains (e.g., cellular or nuclear membrane), cytological stains, or combinations thereof. In some examples, the stain may be specific to proteins, phospholipids, DNA (e.g., dsDNA, ssDNA), RNA, an organelle or compartment of the cell. The sample may be contacted with one or more labeled antibodies (e.g., a primary antibody specific for the analyte of interest and a labeled secondary antibody specific for the primary antibody). In some embodiments, cells in the sample can be segmented using one or more images taken of the stained sample.

In some embodiments, the stain is performed using a lipophilic dye. In some examples, the staining is performed with a lipophilic carbocyanine or aminostyryl dye, or analogs thereof (e.g, DiI, DiO, DiR, DiD). Other cell membrane stains may include FM and RH dyes or immunohistochemical reagents specific for cell membrane proteins. In some examples, the stain may include but not limited to, acridine orange, Bismarck brown, carmine, coomassie blue, cresyl violet, DAPI, eosin, ethidium bromide, acid fuchsine, haematoxylin, Hoechst stains, iodine, methyl green, methylene blue, neutral red, Nile blue, Nile red, osmium tetroxide, ruthenium red, propidium iodide, rhodamine (e.g., rhodamine B), or safranine or derivatives thereof. In some embodiments, the sample may be stained with haematoxylin and eosin (H&E).

The sample can be stained using hematoxylin and eosin (H&E) staining techniques, using Papanicolaou staining techniques, Masson's trichrome staining techniques, silver staining techniques, Sudan staining techniques, and/or using Periodic Acid Schiff (PAS) staining techniques. PAS staining is typically performed after formalin or acetone fixation. In some embodiments, the sample can be stained using Romanowsky stain, including Wright's stain, Jenner's stain, Can-Grunwald stain, Leishman stain, and Giemsa stain. In some embodiments, biological samples can be destained. Any suitable methods of destaining or discoloring a biological sample may be utilized, and generally depend on the nature of the stain(s) applied to the sample. For example, in some embodiments, one or more immunofluorescent stains are applied to the sample via antibody coupling. Such stains can be removed using techniques such as cleavage of disulfide linkages via treatment with a reducing agent and detergent washing, chaotropic salt treatment, treatment with antigen retrieval solution, and treatment with an acidic glycine buffer. Methods for multiplexed staining and destaining are described, for example, in Bolognesi et al., J. Histochem. Cytochem. 2017; 65(8): 431-444, Lin et al., Nat Commun. 2015; 6:8390, Pirici et al., J. Histochem. Cytochem. 2009; 57:567-75, and Glass et al., J. Histochem. Cytochem. 2009; 57:899-905, the entire contents of each of which are incorporated herein by reference. (vi) Isometric Expansion

In some embodiments, a biological sample embedded in a matrix (e.g., a hydrogel) can be isometrically expanded. Isometric expansion methods that can be used include hydration, a preparative step in expansion microscopy, as described in Chen et al., Science 347(6221):543-548, 2015.

Isometric expansion can be performed by anchoring one or more components of a biological sample to a gel, followed by gel formation, proteolysis, and swelling. In some embodiments, analytes in the sample, products of the analytes, and/or probes associated with analytes in the sample can be anchored to the matrix (e.g., hydrogel). Isometric expansion of the biological sample can occur prior to immobilization of the biological sample on a substrate, or after the biological sample is immobilized to a substrate. In some embodiments, the isometrically expanded biological sample can be removed from the substrate prior to contacting the substrate with probes disclosed herein.

In general, the steps used to perform isometric expansion of the biological sample can depend on the characteristics of the sample (e.g., thickness of tissue section, fixation, cross-linking), and/or the analyte of interest (e.g., different conditions to anchor RNA, DNA, and protein to a gel).

In some embodiments, proteins in the biological sample are anchored to a swellable gel such as a polyelectrolyte gel. An antibody can be directed to the protein before, after, or in conjunction with being anchored to the swellable gel. DNA and/or RNA in a biological sample can also be anchored to the swellable gel via a suitable linker. Examples of such linkers include, but are not limited to, 6-((Acryloyl)amino) hexanoic acid (Acryloyl-X SE) (available from ThermoFisher, Waltham, Mass.), Label-IT Amine (available from MirusBio, Madison, Wis.) and Label X (described for example in Chen et al., Nat. Methods 13:679-684, 2016, the entire contents of which are incorporated herein by reference).

Isometric expansion of the sample can increase the spatial resolution of the subsequent analysis of the sample. The increased resolution in spatial profiling can be determined by comparison of an isometrically expanded sample with a sample that has not been isometrically expanded.

In some embodiments, a biological sample is isometrically expanded to a size at least 2×, 2.1×, 2.2×, 2.3×, 2.4×, 2.5×, 2.6×, 2.7×, 2.8×, 2.9×, 3×, 3.1×, 3.2×, 3.3×, 3.4×, 3.5×, 3.6×, 3.7×, 3.8×, 3.9×, 4×, 4.1×, 4.2×, 4.3×, 4.4×, 4.5×, 4.6×, 4.7×, 4.8×, or 4.9× its non-expanded size. In some embodiments, the sample is isometrically expanded to at least 2× and less than 20× of its non-expanded size.

(vii) Crosslinking and De-Crosslinking

In some embodiments, the biological sample is reversibly cross-linked prior to or during an in situ assay round. In some aspects, the analytes, polynucleotides and/or amplification product (e.g., amplicon) of an analyte or a probe bound thereto can be anchored to a polymer matrix. For example, the polymer matrix can be a hydrogel. In some embodiments, one or more of the polynucleotide probe(s) and/or amplification product (e.g., amplicon) thereof can be modified to contain functional groups that can be used as an anchoring site to attach the polynucleotide probes and/or amplification product to a polymer matrix. In some embodiments, a modified probe comprising oligo dT may be used to bind to mRNA molecules of interest, followed by reversible crosslinking of the mRNA molecules.

In some embodiments, the biological sample is immobilized in a hydrogel via cross-linking of the polymer material that forms the hydrogel. Cross-linking can be performed chemically and/or photochemically, or alternatively by any other suitable hydrogel-formation method. A hydrogel may include a macromolecular polymer gel including a network. Within the network, some polymer chains can optionally be cross-linked, although cross-linking does not always occur.

In some embodiments, a hydrogel can include hydrogel subunits, such as, but not limited to, acrylamide, bis-acrylamide, polyacrylamide and derivatives thereof, poly(ethylene glycol) and derivatives thereof (e.g. PEG-acrylate (PEG-DA), PEG-RGD), gelatin-methacryloyl (GelMA), methacrylated hyaluronic acid (MeHA), polyaliphatic polyurethanes, polyether polyurethanes, polyester polyurethanes, polyethylene copolymers, polyamides, polyvinyl alcohols, polypropylene glycol, polytetramethylene oxide, polyvinyl pyrrolidone, polyacrylamide, poly(hydroxyethyl acrylate), and poly(hydroxyethyl methacrylate), collagen, hyaluronic acid, chitosan, dextran, agarose, gelatin, alginate, protein polymers, methylcellulose, and the like, and combinations thereof.

In some embodiments, a hydrogel includes a hybrid material, e.g., the hydrogel material includes elements of both synthetic and natural polymers. Examples of suitable hydrogels are described, for example, in U.S. Pat. Nos. 6,391,937, 9,512,422, and 9,889,422, and in U.S. Patent Application Publication Nos. 2017/0253918, 2018/0052081 and 2010/0055733, the entire contents of each of which are incorporated herein by reference.

In some embodiments, the hydrogel can form the substrate. In some embodiments, the substrate includes a hydrogel and one or more second materials. In some embodiments, the hydrogel is placed on top of one or more second materials. For example, the hydrogel can be pre-formed and then placed on top of, underneath, or in any other configuration with one or more second materials. In some embodiments, hydrogel formation occurs after contacting one or more second materials during formation of the substrate. Hydrogel formation can also occur within a structure (e.g., wells, ridges, projections, and/or markings) located on a substrate.

In some embodiments, hydrogel formation on a substrate occurs before, contemporaneously with, or after probes are provided to the sample. For example, hydrogel formation can be performed on the substrate already containing the probes.

In some embodiments, hydrogel formation occurs within a biological sample. In some embodiments, a biological sample (e.g., tissue section) is embedded in a hydrogel. In some embodiments, hydrogel subunits are infused into the biological sample, and polymerization of the hydrogel is initiated by an external or internal stimulus.

In embodiments in which a hydrogel is formed within a biological sample, functionalization chemistry can be used. In some embodiments, functionalization chemistry includes hydrogel-tissue chemistry (HTC). Any hydrogel-tissue backbone (e.g., synthetic or native) suitable for HTC can be used for anchoring biological macromolecules and modulating functionalization. Non-limiting examples of methods using HTC backbone variants include CLARITY, PACT, ExM, SWITCH and ePACT. In some embodiments, hydrogel formation within a biological sample is permanent. For example, biological macromolecules can permanently adhere to the hydrogel allowing multiple rounds of interrogation. In some embodiments, hydrogel formation within a biological sample is reversible.

In some embodiments, additional reagents are added to the hydrogel subunits before, contemporaneously with, and/or after polymerization. For example, additional reagents can include but are not limited to oligonucleotides (e.g., probes), endonucleases to fragment DNA, fragmentation buffer for DNA, DNA polymerase enzymes, dNTPs used to amplify the nucleic acid and to attach the barcode to the amplified fragments. Other enzymes can be used, including without limitation, RNA polymerase, transposase, ligase, proteinase K, and DNAse. Additional reagents can also include reverse transcriptase enzymes, including enzymes with terminal transferase activity, primers, and switch oligonucleotides. In some embodiments, optical labels are added to the hydrogel subunits before, contemporaneously with, and/or after polymerization.

In some embodiments, HTC reagents are added to the hydrogel before, contemporaneously with, and/or after polymerization. In some embodiments, a cell labeling agent is added to the hydrogel before, contemporaneously with, and/or after polymerization. In some embodiments, a cell-penetrating agent is added to the hydrogel before, contemporaneously with, and/or after polymerization.

Hydrogels embedded within biological samples can be cleared using any suitable method. For example, electrophoretic tissue clearing methods can be used to remove biological macromolecules from the hydrogel-embedded sample. In some embodiments, a hydrogel-embedded sample is stored before or after clearing of hydrogel, in a medium (e.g., a mounting medium, methylcellulose, or other semi-solid mediums).

In some embodiments, a method disclosed herein comprises de-crosslinking the reversibly cross-linked biological sample. The de-crosslinking does not need to be complete. In some embodiments, only a portion of crosslinked molecules in the reversibly cross-linked biological sample are de-crosslinked and allowed to migrate.

(viii) Tissue Permeabilization and Treatment

In some embodiments, a biological sample can be permeabilized to facilitate transfer of analytes out of the sample, and/or to facilitate transfer of species (such as probes) into the sample. If a sample is not permeabilized sufficiently, the amount of analyte captured from the sample may be too low to enable adequate analysis. Conversely, if the tissue sample is too permeable, the relative spatial relationship of the analytes within the tissue sample can be lost. Hence, a balance between permeabilizing the tissue sample enough to obtain good signal intensity while still maintaining the spatial resolution of the analyte distribution in the sample is desirable.

In general, a biological sample can be permeabilized by exposing the sample to one or more permeabilizing agents. Suitable agents for this purpose include, but are not limited to, organic solvents (e.g., acetone, ethanol, and methanol), cross-linking agents (e.g., paraformaldehyde), detergents (e.g., saponin, Triton X-100™ or Tween-20™), and enzymes (e.g., trypsin, proteases). In some embodiments, the biological sample can be incubated with a cellular permeabilizing agent to facilitate permeabilization of the sample. Additional methods for sample permeabilization are described, for example, in Jamur et al., Method Mol. Biol. 588:63-66, 2010, the entire contents of which are incorporated herein by reference. Any suitable method for sample permeabilization can generally be used in connection with the samples described herein.

In some embodiments, the biological sample can be permeabilized by adding one or more lysis reagents to the sample. Examples of suitable lysis agents include, but are not limited to, bioactive reagents such as lysis enzymes that are used for lysis of different cell types, e.g., gram positive or negative bacteria, plants, yeast, mammalian, such as lysozymes, achromopeptidase, lysostaphin, labiase, kitalase, lyticase, and a variety of other commercially available lysis enzymes.

Other lysis agents can additionally or alternatively be added to the biological sample to facilitate permeabilization. For example, surfactant-based lysis solutions can be used to lyse sample cells. Lysis solutions can include ionic surfactants such as, for example, sarcosyl and sodium dodecyl sulfate (SDS). More generally, chemical lysis agents can include, without limitation, organic solvents, chelating agents, detergents, surfactants, and chaotropic agents.

In some embodiments, the biological sample can be permeabilized by non-chemical permeabilization methods. Any suitable non-chemical permeabilization method can be used. For example, non-chemical permeabilization methods that can be used include, but are not limited to, physical lysis techniques such as electroporation, mechanical permeabilization methods (e.g., bead beating using a homogenizer and grinding balls to mechanically disrupt sample tissue structures), acoustic permeabilization (e.g., sonication), and thermal lysis techniques such as heating to induce thermal permeabilization of the sample.

Additional reagents can be added to a biological sample to perform various functions prior to analysis of the sample. In some embodiments, DNase and RNase inactivating agents or inhibitors such as proteinase K, and/or chelating agents such as EDTA, can be added to the sample. For example, a method disclosed herein may comprise a step for increasing accessibility of a nucleic acid for binding, e.g., a denaturation step to opening up DNA in a cell for hybridization by a probe. For example, proteinase K treatment may be used to free up DNA with proteins bound thereto.

### (ix) Selective Enrichment of RNA Species

In some embodiments, where RNA is the analyte, one or more RNA analyte species of interest can be selectively enriched. For example, one or more species of RNA of interest can be selected by addition of one or more oligonucleotides to the sample. In some embodiments, the additional oligonucleotide is a sequence used for priming a reaction by an enzyme (e.g., a polymerase). For example, one or more primer sequences with sequence complementarity to one or more RNAs of interest can be used to amplify the one or more RNAs of interest, thereby selectively enriching these RNAs.

In some embodiments, one or more nucleic acid probes can be used to hybridize to a target nucleic acid (e.g., cDNA or RNA molecule, such as an mRNA) and ligated in a templated ligation reaction (e.g., RNA-templated ligation (RTL) or DNA-templated ligation (e.g., on cDNA)) to generate a product for analysis. In some aspects, when two or more analytes are analyzed, a first and second probe that is specific for (e.g., specifically hybridizes to) each RNA or cDNA analyte are used. For example, in some embodiments of the methods provided herein, templated ligation is used to detect gene expression in a biological sample. An analyte of interest (such as a protein), bound by a labeling agent or binding agent (e.g., an antibody or epitope binding fragment thereof), wherein the binding agent is conjugated or otherwise associated with a reporter oligonucleotide comprising a reporter sequence that identifies the binding agent, can be targeted for analysis. Probes may be hybridized to the reporter oligonucleotide and ligated in a templated ligation reaction to generate a product for analysis. In some embodiments, gaps between the probe oligonucleotides may first be filled prior to ligation, using, for example, Mu polymerase, DNA polymerase, RNA polymerase, reverse transcriptase, VENT polymerase, Taq polymerase, and/or any combinations, derivatives, and variants (e.g., engineered mutants) thereof. In some embodiments, the assay can further include amplification of templated ligation products (e.g., by multiplex PCR).

In some embodiments, an oligonucleotide with sequence complementarity to the complementary strand of captured RNA (e.g., cDNA) can bind to the cDNA. For example, biotinylated oligonucleotides with sequence complementary to one or more cDNA of interest binds to the cDNA and can be selected using biotinylation-strepavidin affinity based methods (e.g., streptavidin beads).

Alternatively, one or more species of RNA can be down-selected (e.g., removed) using any of a variety of methods. For example, probes can be administered to a sample that selectively hybridize to ribosomal RNA (rRNA), thereby reducing the pool and concentration of rRNA in the sample. Additionally and alternatively, duplex-specific nuclease (DSN) treatment can remove rRNA (see, e.g., Archer, et al, Selective and flexible depletion of problematic sequences from RNA-seq libraries at the cDNA stage, BMC Genomics, 15 401, (2014), the entire contents of which are incorporated herein by reference). Furthermore, hydroxyapatite chromatography can remove abundant species (e.g., rRNA) (see, e.g., Vandernoot, V.A., cDNA normalization by hydroxyapatite chromatography to enrich transcriptome diversity in RNA-seq applications, Biotechniques, 53(6) 373-80, (2012), the entire contents of which are incorporated herein by reference).

A biological sample may comprise one or a plurality of analytes of interest. Methods for performing multiplexed assays to analyze two or more different analytes in a single biological sample are provided.

B. Analytes

The methods and compositions disclosed herein can be used to detect and analyze a wide variety of different analytes. In some aspects, an analyte can include any biological substance, structure, moiety, or component to be analyzed. In some aspects, a target disclosed herein may similarly include any analyte of interest. In some examples, a target or analyte can be directly or indirectly detected.

Analytes can be derived from a specific type of cell and/or a specific subcellular region. For example, analytes can be derived from cytosol, from cell nuclei, from mitochondria, from microsomes, and more generally, from any other compartment, organelle, or portion of a cell. Permeabilizing agents that specifically target certain cell compartments and organelles can be used to selectively release analytes from cells for analysis, and/or allow access of one or more reagents (e.g., probes for analyte detection) to the analytes in the cell or cell compartment or organelle.

The analyte may include any biomolecule or chemical compound, including a macromolecule such as a protein or peptide, a lipid or a nucleic acid molecule, or a small molecule, including organic or inorganic molecules. The analyte may be a cell or a microorganism, including a virus, or a fragment or product thereof. An analyte can be any substance or entity for which a specific binding partner (e.g. an affinity binding partner) can be developed. Such a specific binding partner may be a nucleic acid probe (for a nucleic acid analyte) and may lead directly to the generation of a RCA template (e.g. a padlock or other circularizable probe). Alternatively, the specific binding partner may be coupled to a nucleic acid, which may be detected using an RCA strategy, e.g. in an assay which uses or generates a circular nucleic acid molecule which can be the RCA template.

Analytes of particular interest may include nucleic acid molecules, such as DNA (e.g. genomic DNA, mitochondrial DNA, plastid DNA, viral DNA, etc.) and RNA (e.g. mRNA, microRNA, rRNA, snRNA, viral RNA, etc.), and synthetic and/or modified nucleic acid molecules, (e.g. including nucleic acid domains comprising or consisting of synthetic or modified nucleotides such as LNA, PNA, morpholino, etc.), proteinaceous molecules such as peptides, polypeptides, proteins or prions or any molecule which includes a protein or polypeptide component, etc., or fragments thereof, or a lipid or carbohydrate molecule, or any molecule which comprise a lipid or carbohydrate component. The analyte may be a single molecule or a complex that contains two or more molecular subunits, e.g. including but not limited to protein-DNA complexes, which may or may not be covalently bound to one another, and which may be the same or different. Thus in addition to cells or microorganisms, such a complex analyte may also be a protein complex or protein interaction. Such a complex or interaction may thus be a homo- or hetero-multimer. Aggregates of molecules, e.g. proteins may also be target analytes, for example aggregates of the same protein or different proteins. The analyte may also be a complex between proteins or peptides and nucleic acid molecules such as DNA or RNA, e.g. interactions between proteins and nucleic acids, e.g. regulatory factors, such as transcription factors, and DNA or RNA.

(i) Endogenous Analytes

In some embodiments, an analyte herein is endogenous to a biological sample and can include nucleic acid analytes and non-nucleic acid analytes. Methods and compositions disclosed herein can be used to analyze nucleic acid analytes (e.g., using a nucleic acid probe or probe set that directly or indirectly hybridizes to a nucleic acid analyte) and/or non-nucleic acid analytes (e.g., using a labeling agent that comprises a reporter oligonucleotide and binds directly or indirectly to a non-nucleic acid analyte) in any suitable combination.

Examples of non-nucleic acid analytes include, but are not limited to, lipids, carbohydrates, peptides, proteins, glycoproteins (N-linked or O-linked), lipoproteins, phosphoproteins, specific phosphorylated or acetylated variants of proteins, amidation variants of proteins, hydroxylation variants of proteins, methylation variants of proteins, ubiquitylation variants of proteins, sulfation variants of proteins, viral coat proteins, extracellular and intracellular proteins, antibodies, and antigen binding fragments. In some embodiments, the analyte is inside a cell or on a cell surface, such as a transmembrane analyte or one that is attached to the cell membrane. In some embodiments, the analyte can be an organelle (e.g., nuclei or mitochondria). In some embodiments, the analyte is an extracellular analyte, such as a secreted analyte. Exemplary analytes include, but are not limited to, a receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, an extracellular matrix protein, a posttranslational modification (e.g., phosphorylation, glycosylation, ubiquitination, nitrosylation, methylation, acetylation or lipidation) state of a cell surface protein, a gap junction, and an adherens junction.

Examples of nucleic acid analytes include DNA analytes such as single-stranded DNA (ssDNA), double-stranded DNA (dsDNA), genomic DNA, methylated DNA, specific methylated DNA sequences, fragmented DNA, mitochondrial DNA, in situ synthesized PCR products, and RNA/DNA hybrids. The DNA analyte can be a transcript of another nucleic acid molecule (e.g., DNA or RNA such as mRNA) present in a tissue sample.

Examples of nucleic acid analytes also include RNA analytes such as various types of coding and non-coding RNA. Examples of the different types of RNA analytes include messenger RNA (mRNA), including a nascent RNA, a pre-mRNA, a primary-transcript RNA, and a processed RNA, such as a capped mRNA (e.g., with a 5′ 7-methyl guanosine cap), a polyadenylated mRNA (poly-A tail at the 3′ end), and a spliced mRNA in which one or more introns have been removed. Also included in the analytes disclosed herein are non-capped mRNA, a non-polyadenylated mRNA, and a non-spliced mRNA. The RNA analyte can be a transcript of another nucleic acid molecule (e.g., DNA or RNA such as viral RNA) present in a tissue sample. Examples of a non-coding RNAs (ncRNA) that is not translated into a protein include transfer RNAs (tRNAs) and ribosomal RNAs (rRNAs), as well as small non-coding RNAs such as microRNA (miRNA), small interfering RNA (siRNA), Piwi-interacting RNA (piRNA), small nucleolar RNA (snoRNA), small nuclear RNA (snRNA), extracellular RNA (exRNA), small Cajal body-specific RNAs (scaRNAs), and the long ncRNAs such as Xist and HOTAIR. The RNA can be small (e.g., less than 200 nucleic acid bases in length) or large (e.g., RNA greater than 200 nucleic acid bases in length). Examples of small RNAs include 5.8S ribosomal RNA (rRNA), 5S rRNA, tRNA, miRNA, siRNA, snoRNAs, piRNA, tRNA-derived small RNA (tsRNA), and small rDNA-derived RNA (srRNA). The RNA can be double-stranded RNA or single-stranded RNA. The RNA can be circular RNA. The RNA can be a bacterial rRNA (e.g., 16s rRNA or 23s rRNA).

In some embodiments described herein, an analyte may be a denatured nucleic acid, wherein the resulting denatured nucleic acid is single-stranded. The nucleic acid may be denatured, for example, optionally using formamide, heat, or both formamide and heat. In some embodiments, the nucleic acid is not denatured for use in a method disclosed herein.

In certain embodiments, an analyte can be extracted from a live cell. Processing conditions can be adjusted to ensure that a biological sample remains live during analysis, and analytes are extracted from (or released from) live cells of the sample. Live cell-derived analytes can be obtained only once from the sample, or can be obtained at intervals from a sample that continues to remain in viable condition.

Methods and compositions disclosed herein can be used to analyze any number of analytes. For example, the number of analytes that are analyzed can be at least about 2, at least about 3, at least about 4, at least about 5, at least about 6, at least about 7, at least about 8, at least about 9, at least about 10, at least about 11, at least about 12, at least about 13, at least about 14, at least about 15, at least about 20, at least about 25, at least about 30, at least about 40, at least about 50, at least about 100, at least about 1,000, at least about 10,000, at least about 100,000 or more different analytes present in a region of the sample or within an individual feature of the substrate.

In any embodiment described herein, the analyte comprises a target sequence. In some embodiments, the target sequence may be endogenous to the sample, generated in the sample, added to the sample, or associated with an analyte in the sample. In some embodiments, the target sequence is a single-stranded target sequence (e.g., a sequence in a rolling circle amplification product). In some embodiments, the analytes comprise one or more single-stranded target sequences. In one aspect, a first single-stranded target sequence is not identical to a second single-stranded target sequence. In another aspect, a first single-stranded target sequence is identical to one or more second single-stranded target sequence. In some embodiments, the one or more second single-stranded target sequence is comprised in the same analyte (e.g., nucleic acid) as the first single-stranded target sequence. Alternatively, the one or more second single-stranded target sequence is comprised in a different analyte (e.g., nucleic acid) from the first single-stranded target sequence.

(ii) Labeling Agents

In some embodiments, provided herein are methods and compositions for analyzing endogenous analytes (e.g., RNA, ssDNA, and cell surface or intracellular proteins and/or metabolites) in a sample using one or more labeling agents. In some embodiments, an analyte labeling agent may include an agent that interacts with an analyte (e.g., an endogenous analyte in a sample). In some embodiments, the labeling agents can comprise a reporter oligonucleotide that is indicative of the analyte or portion thereof interacting with the labeling agent. For example, the reporter oligonucleotide may comprise a barcode sequence that permits identification of the labeling agent. In some cases, the sample contacted by the labeling agent can be further contacted with a probe (e.g., a single-stranded probe sequence), that hybridizes to a reporter oligonucleotide of the labeling agent, in order to identify the analyte associated with the labeling agent. In some embodiments, the analyte labeling agent comprises an analyte binding moiety and a labeling agent barcode domain comprising one or more barcode sequences, e.g., a barcode sequence that corresponds to the analyte binding moiety and/or the analyte. An analyte binding moiety barcode includes to a barcode that is associated with or otherwise identifies the analyte binding moiety. In some embodiments, by identifying an analyte binding moiety by identifying its associated analyte binding moiety barcode, the analyte to which the analyte binding moiety binds can also be identified. An analyte binding moiety barcode can be a nucleic acid sequence of a given length and/or sequence that is associated with the analyte binding moiety. An analyte binding moiety barcode can generally include any of the variety of aspects of barcodes described herein.

In some embodiments, the method comprises one or more post-fixing (also referred to as post-fixation) steps after contacting the sample with one or more labeling agents.

In the methods and systems described herein, one or more labeling agents capable of binding to or otherwise coupling to one or more features may be used to characterize analytes, cells and/or cell features. In some instances, cell features include cell surface features. Analytes may include, but are not limited to, a protein, a receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, a gap junction, an adherens junction, or any combination thereof. In some instances, cell features may include intracellular analytes, such as proteins, protein modifications (e.g., phosphorylation status or other post-translational modifications), nuclear proteins, nuclear membrane proteins, or any combination thereof.

In some embodiments, an analyte binding moiety may include any molecule or moiety capable of binding to an analyte (e.g., a biological analyte, e.g., a macromolecular constituent). A labeling agent may include, but is not limited to, a protein, a peptide, an antibody (or an epitope binding fragment thereof), a lipophilic moiety (such as cholesterol), a cell surface receptor binding molecule, a receptor ligand, a small molecule, a bi-specific antibody, a bi-specific T-cell engager, a T-cell receptor engager, a B-cell receptor engager, a pro-body, an aptamer, a monobody, an affimer, a darpin, and a protein scaffold, or any combination thereof. The labeling agents can include (e.g., are attached to) a reporter oligonucleotide that is indicative of the cell surface feature to which the binding group binds. For example, the reporter oligonucleotide may comprise a barcode sequence that permits identification of the labeling agent. For example, a labeling agent that is specific to one type of cell feature (e.g., a first cell surface feature) may have coupled thereto a first reporter oligonucleotide, while a labeling agent that is specific to a different cell feature (e.g., a second cell surface feature) may have a different reporter oligonucleotide coupled thereto. For a description of exemplary labeling agents, reporter oligonucleotides, and methods of use, see, e.g., U.S. Pat. No. 10,550,429; U.S. Pat. Pub. 20190177800; and U.S. Pat. Pub. 20190367969, which are each incorporated by reference herein in their entirety.

In some embodiments, an analyte binding moiety includes one or more antibodies or antigen binding fragments thereof. The antibodies or antigen binding fragments including the analyte binding moiety can specifically bind to a target analyte. In some embodiments, the analyte is a protein (e.g., a protein on a surface of the biological sample (e.g., a cell) or an intracellular protein). In some embodiments, a plurality of analyte labeling agents comprising a plurality of analyte binding moieties bind a plurality of analytes present in a biological sample. In some embodiments, the plurality of analytes includes a single species of analyte (e.g., a single species of polypeptide). In some embodiments in which the plurality of analytes includes a single species of analyte, the analyte binding moieties of the plurality of analyte labeling agents are the same. In some embodiments in which the plurality of analytes includes a single species of analyte, the analyte binding moieties of the plurality of analyte labeling agents are the different (e.g., members of the plurality of analyte labeling agents can have two or more species of analyte binding moieties, wherein each of the two or more species of analyte binding moieties binds a single species of analyte, e.g., at different binding sites). In some embodiments, the plurality of analytes includes multiple different species of analyte (e.g., multiple different species of polypeptides).

In other instances, e.g., to facilitate sample multiplexing, a labeling agent that is specific to a particular cell feature may have a first plurality of the labeling agent (e.g., an antibody or lipophilic moiety) coupled to a first reporter oligonucleotide and a second plurality of the labeling agent coupled to a second reporter oligonucleotide.

In some aspects, these reporter oligonucleotides may comprise nucleic acid barcode sequences that permit identification of the labeling agent which the reporter oligonucleotide is coupled to. The selection of oligonucleotides as the reporter may provide advantages of being able to generate significant diversity in terms of sequence, while also being readily attachable to most biomolecules, e.g., antibodies, etc., as well as being readily detected, e.g., using sequencing or array technologies.

Attachment (coupling) of the reporter oligonucleotides to the labeling agents may be achieved through any of a variety of direct or indirect, covalent or non-covalent associations or attachments. For example, oligonucleotides may be covalently attached to a portion of a labeling agent (such a protein, e.g., an antibody or antibody fragment) using chemical conjugation techniques (e.g., Lightning-Link® antibody labeling kits available from Innova Biosciences), as well as other non-covalent attachment mechanisms, e.g., using biotinylated antibodies and oligonucleotides (or beads that include one or more biotinylated linker, coupled to oligonucleotides) with an avidin or streptavidin linker. Antibody and oligonucleotide biotinylation techniques are available. See, e.g., Fang, et al., “Fluoride-Cleavable Biotinylation Phosphoramidite for 5′-end-Labeling and Affinity Purification of Synthetic Oligonucleotides,” Nucleic Acids Res. Jan. 15, 2003; 31(2):708-715, which is entirely incorporated herein by reference for all purposes. Likewise, protein and peptide biotinylation techniques have been developed and are readily available. See, e.g., U.S. Pat. No. 6,265,552, which is entirely incorporated herein by reference for all purposes. Furthermore, click reaction chemistry may be used to couple reporter oligonucleotides to labeling agents. Commercially available kits, such as those from Thunderlink and Abcam, and techniques common in the art may be used to couple reporter oligonucleotides to labeling agents as appropriate. In another example, a labeling agent is indirectly (e.g., via hybridization) coupled to a reporter oligonucleotide comprising a barcode sequence that identifies the label agent. For instance, the labeling agent may be directly coupled (e.g., covalently bound) to a hybridization oligonucleotide that comprises a sequence that hybridizes with a sequence of the reporter oligonucleotide. Hybridization of the hybridization oligonucleotide to the reporter oligonucleotide couples the labeling agent to the reporter oligonucleotide. In some embodiments, the reporter oligonucleotides are releasable from the labeling agent, such as upon application of a stimulus. For example, the reporter oligonucleotide may be attached to the labeling agent through a labile bond (e.g., chemically labile, photolabile, thermally labile, etc.) as generally described for releasing molecules from supports elsewhere herein. In some instances, the reporter oligonucleotides described herein may include one or more functional sequences that can be used in subsequent processing, such as an adapter sequence, a unique molecular identifier (UMI) sequence, a sequencer specific flow cell attachment sequence (such as an P5, P7, or partial P5 or P7 sequence), a primer or primer binding sequence, a sequencing primer or primer biding sequence (such as an R1, R2, or partial R1 or R2 sequence).

In some cases, the labeling agent can comprise a reporter oligonucleotide and a label. A label can be fluorophore, a radioisotope, a molecule capable of a colorimetric reaction, a magnetic particle, or any other suitable molecule or compound capable of detection. The label can be conjugated to a labeling agent (or reporter oligonucleotide) either directly or indirectly (e.g., the label can be conjugated to a molecule that can bind to the labeling agent or reporter oligonucleotide). In some cases, a label is conjugated to a first oligonucleotide that is complementary (e.g., hybridizes) to a sequence of the reporter oligonucleotide.

In some embodiments, multiple different species of analytes (e.g., polypeptides) from the biological sample can be subsequently associated with the one or more physical properties of the biological sample. For example, the multiple different species of analytes can be associated with locations of the analytes in the biological sample. Such information (e.g., proteomic information when the analyte binding moiety(ies) recognizes a polypeptide(s)) can be used in association with other spatial information (e.g., genetic information from the biological sample, such as DNA sequence information, transcriptome information (e.g., sequences of transcripts), or both). For example, a cell surface protein of a cell can be associated with one or more physical properties of the cell (e.g., a shape, size, activity, or a type of the cell). The one or more physical properties can be characterized by imaging the cell. The cell can be bound by an analyte labeling agent comprising an analyte binding moiety that binds to the cell surface protein and an analyte binding moiety barcode that identifies that analyte binding moiety. Results of protein analysis in a sample (e.g., a tissue sample or a cell) can be associated with DNA and/or RNA analysis in the sample.

C. Target Sequences

A target sequence for a nucleic acid probe disclosed herein (e.g., circularizable probe such as a padlock probe) may be comprised in any analyte disclose herein, including an endogenous analyte (e.g., a specific protein), a labeling agent, or a product of an endogenous analyte and/or a labeling agent.

In some aspects, one or more of the target sequences includes one or more barcode(s), e.g., at least two, three, four, five, six, seven, eight, nine, ten, or more barcodes. Barcodes can spatially-resolve molecular components found in biological samples, for example, within a cell or a tissue sample. A barcode can be attached to an analyte or to another moiety or structure in a reversible or irreversible manner. A barcode can be added to, for example, a fragment of a deoxyribonucleic acid (DNA) or ribonucleic acid (RNA) sample before or during sequencing of the sample. Barcodes can allow for identification and/or quantification of individual sequencing-reads (e.g., a barcode can be or can include a unique molecular identifier or “UMI”). In some aspects, a barcode comprises about 4, 5, 6, 7, 8, 9, 10, 11, 12, 13, 14, 15, 16, 17, 18, 19, 20, 21, 22, 23, 24, 25, 26, 27, 28, 29, 30, or more than 30 nucleotides.

In some embodiments, a barcode includes two or more sub-barcodes that together function as a single barcode. For example, a polynucleotide barcode can include two or more polynucleotide sequences (e.g., sub-barcodes) that are separated by one or more non-barcode sequences. In some embodiments, the one or more barcode(s) can also provide a platform for targeting functionalities, such as oligonucleotides, oligonucleotide-antibody conjugates, oligonucleotide-streptavidin conjugates, modified oligonucleotides, affinity purification, detectable moieties, enzymes, enzymes for detection assays or other functionalities, and/or for detection and identification of the polynucleotide.

In any of the preceding embodiments, barcodes (e.g., primary and/or secondary barcode sequences) can be analyzed (e.g., detected or sequenced) using any suitable methods or techniques, including those described herein, such as RNA sequential probing of targets (RNA SPOTs), sequential fluorescent in situ hybridization (seqFISH), single-molecule fluorescent in situ hybridization (smFISH), multiplexed error-robust fluorescence in situ hybridization (MERFISH), in situ sequencing, hybridization-based in situ sequencing (HybISS), targeted in situ sequencing, fluorescent in situ sequencing (FISSEQ), sequencing by synthesis (SBS), sequencing by ligation (SBL), sequencing by hybridization (SBH), or spatially-resolved transcript amplicon readout mapping (STARmap). In any of the preceding embodiments, the methods provided herein can include analyzing the barcodes by sequential hybridization and detection with a plurality of labelled probes (e.g., detection oligos).

In some embodiments, in a barcode sequencing method, barcode sequences are detected for identification of other molecules including nucleic acid molecules (DNA or RNA) longer than the barcode sequences themselves, as opposed to direct sequencing of the longer nucleic acid molecules. In some embodiments, a N-mer barcode sequence comprises 4′ complexity given a sequencing read of N bases, and a much shorter sequencing read may be required for molecular identification compared to non-barcode sequencing methods such as direct sequencing. For example, 1024 molecular species may be identified using a 5-nucleotide barcode sequence (4⁵=1024), whereas 8 nucleotide barcodes can be used to identify up to 65,536 molecular species, a number greater than the total number of distinct genes in the human genome. In some embodiments, the barcode sequences contained in the probes or RCPs are detected, rather than endogenous sequences, which can be an efficient read-out in terms of information per cycle of sequencing. Because the barcode sequences are pre-determined, they can also be designed to feature error detection and correction mechanisms, see, e.g., U.S. Pat. Pub. 20190055594 and WO2019199579A1, US20210164039, which are hereby incorporated by reference in their entirety.

### VII. In Situ Assay Modules

In some aspects, provided herein are methods comprising in situ assays using microscopy as a readout, e.g., nucleic acid sequencing, hybridization, or other detection or determination methods involving an optical readout. In some aspects, detection or determination of a sequence of one, two, three, four, five, or more nucleotides of a target nucleic acid is performed in situ in a cell in an intact tissue. In some embodiments, the assay comprises detecting the presence or absence of an amplification product (e.g., RCA product). In some embodiments, the present disclosure provides methods for high-throughput profiling of a large number of targets in situ, such as transcripts and/or DNA loci, e.g., for detecting and/or quantifying nucleic acids and/or proteins in cells, tissues, organs or organisms. In some embodiments, the hybridization of probes with the sample and/or detection steps during the in situ assay is performed on analytes in the sample that are not captured by capture probes or capture agents.

A. In Situ Analysis

In some aspects, provided herein is a method comprising analyzing biological targets based on in situ hybridization of probes comprising nucleic acid sequences. In some embodiments, the method comprises sequential hybridization of detectably-labelled oligonucleotides to barcoded probes that directly or indirectly bind to biological targets in a sample. In some embodiments, a detectably-labelled oligonucleotide directly binds to one or more barcoded probes. In some embodiments, a detectably-labelled oligonucleotide indirectly binds to one or more barcoded probes, e.g., via one or more bridging nucleic acid molecules.

In some aspects, an in situ hybridization based assay is used to localize and analyze nucleic acid sequences (e.g., a DNA or RNA molecule comprising one or more specific sequences of interest) within a native biological sample, e.g., a portion or section of tissue or a single cell. In some embodiments, the in situ assay is used to analyze the presence, absence, an amount or level of mRNA transcripts (e.g., a transcriptome or a subset thereof, or mRNA molecules of interest) in a biological sample, while preserving spatial context. In some embodiments, the present disclosure provides compositions and methods for in situ hybridization using directly or indirectly labeled molecules, e.g., complementary DNA or RNA or modified nucleic acids, as probes that bind or hybridize to a target nucleic acids within a biological sample of interest.

Nucleic acid probes, in some examples, may be labelled with radioisotopes, epitopes, hapten, biotin, or fluorophores, to enable detection of the location of specific nucleic acid sequences on chromosomes or in tissues. In some embodiments, probes are locus specific (e.g., gene specific) and bind or couple to specific regions of a chromosome. In alternative embodiments, probes are alphoid or centromeric repeat probes that bind or couple to repetitive sequences within each chromosome. Probes may also be whole chromosome probes (e.g., multiple smaller probes) that bind or couple to sequences along an entire chromosome.

In some embodiments, provided herein is a method comprising DNA in situ hybridization to measure and localize DNA. In some embodiments, provided herein is a method RNA in situ hybridization to measure and localize RNAs (e.g., mRNAs, lncRNAs, and miRNAs) within a biological sample (e.g., a fixed tissue sample). In some embodiments, RNA in situ hybridization involves single-molecule RNA fluorescence in situ hybridization (FISH). In some embodiments, fluorescently labelled nucleic acid probes are hybridized to pre-determined RNA targets, to visualize gene expression in a biological sample. In some embodiments, a FISH method comprises using a single nucleic acid probe specific to each target, e.g., single-molecule FISH (smFISH). The use of smFISH may produce a fluorescence signal that allows for quantitative measurement of RNA transcripts. In some embodiments, smFISH comprises a set of nucleic acid probes, about 50 base pairs in length, wherein each probe is coupled to a set fluorophores. For example, the set of nucleic acid probes may comprise five probes, wherein each probe coupled to five fluorophores. In some embodiments, said nucleic acid probes are instead each coupled to one fluorophore. For example, a smFISH protocol may use a set of about 40 nucleic acid probes, about 20 base pairs in length, each coupled to a single fluorophore. In some embodiments, the length of the nucleic acid probes varies, comprising 10 to 100 base pairs, such as 30 to 60 base pairs. Alternatively, a plurality of nucleic acid probes targeting different regions of the same RNA transcript may be used. It will be appreciated by those skilled in the art that the type of nucleic acid probes, the number of nucleic acid probes, the number of fluorophores coupled to said probes, and the length of said probes, may be varied to fit the specifications of the individual assay.

In further embodiments smFISH is applied to a multiplexed workflow, wherein consecutive/sequential hybridizations are used (e.g., as in seqFISH or seqFISH+) to impart a temporal barcode on target transcripts. Sequential rounds of fluorescence in situ hybridization may be accompanied by imaging and probe stripping, detecting individual transcripts (e.g., RNA transcripts) within a biological sample of interest (e.g., a tissue sample, a single cell, or extracted RNA). In some embodiments, each round of hybridization comprises a pre-defined set of probes (e.g., between about 10 and about 50 probes such as 24 to 32 probes) that target unique RNA transcripts. In some examples, the pre-defined set of probes is multicolored. Optionally, multiple nucleic acid probes are attached onto the sample, wherein each probe comprises an initiation sequence for amplification, allowing for decreased autofluorescence (e.g., as in single-molecule hybridization chain reaction (smHCR)). In some embodiments, a multiplexed smFISH method described herein may multiplex from 10s to over 10,000 mRNAs, optionally accompanied by imaging, to efficiently and accurately profile the entire transcriptome. In situ hybridization methods may further comprise using two probes to bind target transcripts (e.g., RNA transcripts), that serve as binding targets for amplification primers. In some embodiments, this process results in signal amplification (e.g., as in RNAscope). In some embodiments, in situ hybridization methods may employ metal tags instead of fluorophores (e.g., imaging mass cytometry). Metal-conjugated antibodies may couple to the metal tags hybridized to transcripts on a biological sample. In some embodiments, mass-cytometry may be used to quantify metal abundances, allowing the concurrent evaluation of RNA and protein within a biological sample.

In some embodiments, a method described herein comprises a multiplexed FISH protocol that is error-robust (e.g., MERFISH). In some embodiments, said protocol comprises non-readout nucleic acid probes (e.g., primary probes) comprising a binding region (e.g., a region that binds to a target such as RNA transcripts) coupled to one or more flanking regions. In some embodiments, each non-readout nucleic acid probe is coupled to two flanking regions. The non-readout nucleic acid probes may hybridize to a transcript (e.g., RNA transcript) within a biological sample (e.g., tissue sample or a single cell), such that florescent readout nucleic acid probes may subsequently serially hybridize to the flanking region(s) of the non-readout nucleic acid probes. In some embodiments, each round of hybridization comprises successive imaging and probe stripping to quench signals from readout nucleic acid probes from previous rounds. RNAs may be imaged by FISH, and errors accumulated during multiple imaging rounds (e.g., imperfect hybridizations) are detected and/or corrected. In some embodiments, expansion microscopy is employed to increase the number of detected RNA targets without signal overlap. In similar embodiments, non-readout nucleic acid probes are cross-linked to target transcripts prior to imaging. Cross-linking may be performed by any suitable method. In preferred embodiments, cross-linking is performed using hydrogel tissue embedding. Following said cross-linking steps, barcoding may be performed, comprising sequential hybridizations using readout probes coupled to pre-determined colors to generate unique barcodes (e.g., generating pseudocolors from consecutive hybridizations).

In some embodiments, one or more barcodes of a probe are targeted by detectably labeled detection oligonucleotides, such as fluorescently labeled oligonucleotides. In some embodiments, one or more decoding schemes are used to decode the signals, such as fluorescence, for sequence determination. In any of the embodiments herein, barcodes (e.g., primary and/or secondary barcode sequences) can be analyzed using any suitable methods or techniques, comprising those described herein, such as RNA sequential probing of targets (RNA SPOTs), sequential fluorescent in situ hybridization (seqFISH or seqFISH+), single-molecule fluorescent in situ hybridization (smFISH), or multiplexed error-robust fluorescence in situ hybridization (MERFISH). In some embodiments, the methods provided herein comprise analyzing the barcodes by sequential hybridization and detection with a plurality of labelled probes (e.g., detection oligonucleotides). Exemplary decoding schemes are described in Eng et al., “Transcriptome-scale Super-Resolved Imaging in Tissues by RNA SeqFISH+,” Nature 568(7751):235-239 (2019); Chen et al., “Spatially resolved, highly multiplexed RNA profiling in single cells,” Science; 348(6233):aaa6090 (2015); U.S. Pat. No. 10,457,980 B2; US 2016/0369329 A1; WO 2018/026873 A1; US 2021/0017587; and US 2017/0220733 A1, all of which are incorporated by reference in their entirety. In some embodiments, these assays enable signal amplification, combinatorial decoding, and error correction schemes at the same time.

Similar strategies of in situ hybridization using variations of FISH techniques may also be adopted by methods described herein. In some embodiments, a method comprises non-barcoding multiplexed FISH protocols (e.g., ouroboros sm-FISH (osmFISH)). Non-barcoding methods may be limited to detecting a specific number of targets, defined by the number of hybridization rounds performed. In some embodiments, imaging is performed following each hybridization round, wherein the probe is stripped after imaging, allowing for subsequent hybridization and imaging rounds.

Additional embodiments of the present disclosure may include using in situ hybridization protocols that do not rely on probe capture of transcripts from pre-defined locations. In some embodiments, optics-free spatial mapping of transcripts in a biological sample may be used (e.g., a chemically encoded microscopy system). In some embodiments, transcripts are first tagged in situ with unique nucleotide tags (e.g., unique molecular identifiers). This first reaction may be followed by a second in situ amplification reaction, labelled by a new set of unique nucleotide tags (e.g., unique event identifiers). In some embodiments, RNA or DNA sequencing may be used to identify each molecular chain sequence (e.g., concatemers). In further embodiments, an algorithm may be used to evaluate the proximities of the sequences and produce images of the target transcripts, in combination with sequence information.

In some embodiments, provided herein is a method comprising linking sequencing information and spatial information of targets within endogenous environments. For example, analysis of nucleic acid sequences may be performed directly on DNA or RNA within an intact biological sample of interest, e.g., by in situ sequencing. In some embodiments, the present disclosure allows for the simultaneous identification and quantification of a plurality of targets, such as 100s, 1000s, or more of transcripts (e.g., mRNA transcripts), in addition to spatial resolution of said transcripts. In some aspects, the spatial resolution of transcripts may be subcellular. Optionally, the spatial resolution may be increased using signal amplification strategies described herein.

In some embodiments, fluorescent dyes are used to target nucleic acid bases, and padlock probes are used to target RNAs of interest in situ. In some embodiments, mRNAs are reverse transcribed into cDNAs, and padlock probes are able to bind or couple to cDNAs. In some embodiments, padlock probes comprise oligonucleotides with ends that are complementary to a target sequence (e.g., target cDNA transcripts). Upon hybridization of padlock probes to the target sequence, enzymes may be used to ligate the ends of the padlock probes, and catalyze the formation of circularized DNA.

In some embodiments, the ends of the padlock probes are in close proximity upon hybridization to the target RNA or cDNA, to allow ligation and circularization of the padlock probe. The padlock probes may additionally comprise one or more barcode sequences. In alternative embodiments, there may be a gap between the ends of the padlock probes upon hybridization to the target RNA or cDNA, that must be filled with nucleic acids (e.g., by DNA polymerization), prior to ligation of the ends of the padlock probes and circularization. In some embodiments, the gap between to ends of the padlock probes is of variable length, e.g., up to four base pairs, and can allow reading out the actual RNA or cDNA sequence. In some embodiments, the DNA polymerase has strand displacement activity. In some embodiments, the DNA polymerase may instead not have strand displacement activity, such as the polymerase used in barcode in situ target sequencing (BaristaSeq) which provides read-length of up to 15 bases using a gap-filling padlock probe approach. See, e.g., Chen et al., Nucleic Acids Res. 2018, 46, e22, incorporated herein by reference in its entirety.

A method described herein may comprise DNA circularization and amplification (e.g., rolling circle amplification), at the location of padlock probes. In some embodiments, amplification results in multiple repeats of padlock probe sequences. Sequencing and/or decoding of the amplified padlock probes may be performed using sequencing-by-ligation. In alternative methods, sequencing-by-hybridization or sequencing-by-synthesis are used. In some embodiments, amplicons are stabilized by crossing-linking described herein, during the sequencing process. In some embodiments, the in situ sequencing methods presented in this disclosure may be automated on a microfluidic platform.

Additional approaches to in situ sequencing will be appreciated by those skilled in the art. For example, in some embodiments, barcoded padlocks probes may not be reverse transcribed. Instead, a second primer binds (e.g., ligates) directly to an RNA sequence adjacent to the padlock probe. In some embodiments, amplification (e.g., rolling circle amplification) is performed, wherein the amplification product becomes embedded within a hydrogel by any suitable method (e.g., hydrogel-tissue chemistry), which is then cleaned of unbound proteins and lipids. Embedded amplification products may, for example, be sequenced using variations of the sequencing-by-ligation approach, to determine the barcode sequence of each padlock probe. In some embodiments, the combinations of chemistry and sequencing described herein may be used to analyze spatial orientation of target transcripts in 3D.

In some embodiments, an in situ sequencing methods described in the present disclosure may be untargeted. In some embodiments, untargeted in situ sequencing may comprise genome/transcriptome-wide profiling of gene expression within a biological sample of interest, e.g., as in fluorescent in situ RNA sequencing (FISSEQ). In some embodiments, RNA species are captured and converted into cross-linked cDNA amplicons (e.g., cDNA cross-linked to the cellular protein matrix of the sample). In some examples, cDNA synthesis is performed using modified amine bases to promote the cross-linking process. The synthesis of cross-linked cDNA amplicons may be followed by amplification (e.g., rolling circle amplification) as described elsewhere herein. In some embodiments, sequencing-by-ligation may be used to sequence the amplification products. In some embodiments, the sequencing step includes partition sequencing to selectively sequence of subsets of amplification products. In some embodiments, the strategies described herein allow for the detection of RNA, DNA, and/or proteins, in tandem. In some embodiments, in situ sequencing may be combined with ex situ sequencing, e.g., as in in situ transcriptome accessibility sequencing (INSTA-Seq).

In some embodiments, in situ sequencing involves incorporation of a labeled nucleotide (e.g., fluorescently labeled mononucleotides or dinucleotides) in a sequential, template-dependent manner or hybridization of a labeled primer (e.g., a labeled random hexamer) to a nucleic acid template such that the identities (e.g., nucleotide sequence) of the incorporated nucleotides or labeled primer extension products can be determined, and consequently, the nucleotide sequence of the corresponding template nucleic acid. Aspects of in situ sequencing are described, for example, in Mitra et al., (2003) Anal. Biochem. 320, 55-65, and Lee et al., (2014) Science, 343(6177), 1360-1363. In addition, examples of methods and systems for performing in situ sequencing are described in U.S. Pat. No. 11,299,767, US2016/0024555, U.S. Pat. No. 11,085,072, US 2019/0194709, and in U.S. Pat. Nos. 10,138,509 and 10,179,932. Exemplary techniques for in situ sequencing comprise, but are not limited to, STARmap (described for example in Wang et al., (2018) Science, 361(6499) 5691), MERFISH (described for example in Moffitt, (2016) Methods in Enzymology, 572, 1-49), and FISSEQ (described for example in US 2019/0032121).

### (i) Probes and Probe Hybridization

In some aspects, the methods disclosed herein involve the use of one or more probes or probe sets that hybridize to a target nucleic acid, such as an RNA molecule. Exemplary probes or probe sets may be based on a padlock probe, a gapped padlock probe, a SNAIL (Splint Nucleotide Assisted Intramolecular Ligation) probe set, a PLAYR (Proximity Ligation Assay for RNA) probe set, a PLISH (Proximity Ligation in situ Hybridization) probe set, and RNA-templated ligation probes. The specific probe or probe set design can vary. In some embodiments, a primary probe (e.g., a DNA probe that directly binds to an RNA target) is amplified through rolling circle amplification, e.g., using a circular probe or a circularized probe from padlock ligation as a template. In some embodiments, the primary probes, such as a padlock probe or a probe set that comprises a padlock probe, contain one or more barcodes. In some embodiments, one or more barcodes are indicative of a sequence in the target nucleic acid, such as a single nucleotide of interest (e.g., SNPs or point mutations), a dinucleotide sequence, a short sequence of about 5 nucleotides in length, or a sequence of any suitable length.

In some embodiments, provided herein is a probe or probe set capable of DNA-templated ligation, such as from a cDNA molecule. See, e.g., U.S. Pat. No. 8,551,710, which is hereby incorporated by reference in its entirety. In some embodiments, provided herein is a probe or probe set capable of RNA-templated ligation. See, e.g., PCT App. PCT/EP2018/077161, published as WO2019068880 and US20200224244, which are hereby incorporated by reference in its entirety. In some embodiments, the probe set is a SNAIL probe set. See, e.g., U.S. Pat. Pub. 20190055594, which is hereby incorporated by reference in its entirety. In some embodiments, provided herein is a probe or probe set capable of proximity ligation, for instance a proximity ligation assay for RNA (e.g., PLAYR) probe set. See, e.g., U.S. Pat. Pub. 20160108458, which is hereby incorporated by reference in its entirety.

In some embodiments, a circular probe can be indirectly hybridized to the target nucleic acid. In some embodiments, the circular construct is formed from a probe set capable of proximity ligation, for instance a proximity ligation in situ hybridization (PUSH) probe set. See, e.g., PCT App. PCT/US2018/023846, published as WO2018175779, and US20200224243 which are hereby incorporated by reference in its entirety.

### (ii) Ligation

In some embodiments, the provided methods involve ligating one or more polynucleotides that are part of a hybridization complex that comprises a target nucleic acid for in situ analysis. In some embodiments, the ligation involves chemical ligation. In some embodiments, the ligation involves template dependent ligation. In some embodiments, the ligation involves template independent ligation. In some embodiments, the ligation involves enzymatic ligation.

In some embodiments, the enzymatic ligation involves use of a ligase. In some aspects, the ligase used herein comprises an enzyme that is commonly used to join polynucleotides together or to join the ends of a single polynucleotide. An RNA ligase, a DNA ligase, or another variety of ligase can be used to ligate two nucleotide sequences together. Ligases comprise ATP-dependent double-strand polynucleotide ligases, NAD-i-dependent double-strand DNA or RNA ligases and single-strand polynucleotide ligases, for example any of the ligases described in EC 6.5.1.1 (ATP-dependent ligases), EC 6.5.1.2 (NAD+-dependent ligases), EC 6.5.1.3 (RNA ligases). Specific examples of ligases comprise bacterial ligases such as E. coli DNA ligase, Tth DNA ligase, Thermococcus sp. (strain 9° N) DNA ligase (9° N™ DNA ligase, New England Biolabs), Taq DNA ligase, Ampligase™ (Epicentre Biotechnologies) and phage ligases such as T3 DNA ligase, T4 DNA ligase and T7 DNA ligase and mutants thereof. In some embodiments, the ligase is a T4 RNA ligase. In some embodiments, the ligase is a splintR ligase. In some embodiments, the ligase is a single stranded DNA ligase. In some embodiments, the ligase is a T4 DNA ligase. In some embodiments, the ligase is a ligase that has an DNA-splinted DNA ligase activity. In some embodiments, the ligase is a ligase that has an RNA-splinted DNA ligase activity.

(iii) Amplification

In some embodiments, the methods of the invention comprise the step of amplifying one or more polynucleotides, for instance the padlock probe or a circular probe formed from the padlock probe. In some embodiments, the amplifying is achieved by performing rolling circle amplification (RCA). In other embodiments, a primer that hybridizes to the padlock probe is added and used as such for amplification.

In some embodiments, a removing step is performed to remove molecules that are not specifically hybridized to the target nucleic acid and/or the circular probe. In some embodiments, the removing step is performed to remove unligated probes. In some embodiments, the removing step is performed after ligation and prior to amplification.

### (iv) Detection and Analysis

In some embodiments, sequencing can be performed by sequencing-by-synthesis (SBS). In some embodiments, a sequencing primer is complementary to sequences at or near the one or more barcode(s). In such embodiments, sequencing-by-synthesis can comprise reverse transcription and/or amplification in order to generate a template sequence from which a primer sequence can bind. Exemplary SBS methods comprise those described for example, but not limited to, US 2007/0166705, US 2006/0188901, U.S. Pat. No. 7,057,026, US 2006/0240439, US 2006/0281109, U.S. Pat. No. 8,563,477, US 2005/0100900, WO 06/064199, U.S. Pat. No. 8,715,966, US 2012/0270305, US 2013/0260372, and US 2013/0079232.

In some embodiments, sequencing can be performed by sequential fluorescence hybridization (e.g., sequencing by hybridization). Sequential fluorescence hybridization can involve sequential hybridization of detection probes comprising an oligonucleotide and a detectable label.

In some embodiments, sequencing can be performed using single molecule sequencing by ligation. Such techniques utilize DNA ligase to incorporate oligonucleotides and identify the incorporation of such oligonucleotides. The oligonucleotides typically have different labels that are correlated with the identity of a particular nucleotide in a sequence to which the oligonucleotides hybridize. Aspects and features involved in sequencing by ligation are described, for example, in Shendure et al. Science (2005), 309: 1728-1732, and in U.S. Pat. Nos. 5,599,675; 5,750,341; 6,969,488; 6,172,218; and 6,306,597.

In some embodiments, the barcodes of the detection probes are targeted by detectably labeled detection oligonucleotides, such as fluorescently labeled oligonucleotides. In some embodiments, one or more decoding schemes are used to decode the signals, such as fluorescence, for sequence determination. In any of the embodiments herein, barcodes (e.g., primary and/or secondary barcode sequences) can be analyzed (e.g., detected or sequenced) using any suitable methods or techniques, comprising those described herein, such as RNA sequential probing of targets (RNA SPOTs), sequential fluorescent in situ hybridization (seqFISH), single-molecule fluorescent in situ hybridization (smFISH), multiplexed error-robust fluorescence in situ hybridization (MERFISH), hybridization-based in situ sequencing (HybISS), in situ sequencing, targeted in situ sequencing, fluorescent in situ sequencing (FISSEQ), or spatially-resolved transcript amplicon readout mapping (STARmap). In some embodiments, the methods provided herein comprise analyzing the barcodes by sequential hybridization and detection with a plurality of labelled probes (e.g., detection oligonucleotides). Exemplary decoding schemes are described in Eng et al., “Transcriptome-scale Super-Resolved Imaging in Tissues by RNA SeqFISH+,” Nature 568(7751):235-239 (2019); Chen et al., “Spatially resolved, highly multiplexed RNA profiling in single cells,” Science; 348(6233):aaa6090 (2015); U.S. Pat. No. 10,457,980 B2; US 2016/0369329 A1; WO 2018/026873 A1; US 2021/0017587; and US 2017/0220733 A1, all of which are incorporated by reference in their entirety. In some embodiments, these assays enable signal amplification, combinatorial decoding, and error correction schemes at the same time.

In some embodiments, nucleic acid hybridization can be used for sequencing. These methods utilize labeled nucleic acid decoder probes that are complementary to at least a portion of a barcode sequence. Multiplex decoding can be performed with pools of many different probes with distinguishable labels. Non-limiting examples of nucleic acid hybridization sequencing are described for example in U.S. Pat. No. 8,460,865, and in Gunderson et al., Genome Research 14:870-877 (2004).

In some embodiments, real-time monitoring of DNA polymerase activity can be used during sequencing. For example, nucleotide incorporations can be detected through fluorescence resonance energy transfer (FRET), as described for example in Levene et al., Science (2003), 299, 682-686, Lundquist et al., Opt. Lett. (2008), 33, 1026-1028, and Korlach et al., Proc. Natl. Acad. Sci. USA (2008), 105, 1176-1181.

In some aspects, the analysis and/or sequence determination can be carried out at room temperature for best preservation of tissue morphology with low background noise and error reduction. In some embodiments, the analysis and/or sequence determination comprises eliminating error accumulation as sequencing proceeds.

In some embodiments, the analysis and/or sequence determination involves washing to remove unbound polynucleotides, thereafter revealing a fluorescent product for imaging.

In some aspects, the detection (comprising imaging) is carried out using any of a number of different types of microscopy, e.g., confocal microscopy, two-photon microscopy, light-field microscopy, intact tissue expansion microscopy, and/or CLARITY™-optimized light sheet microscopy (COLM).

In some embodiments, fluorescence microscopy is used for detection and imaging of the detection probe. In some aspects, a fluorescence microscope is an optical microscope that uses fluorescence and phosphorescence instead of, or in addition to, reflection and absorption to study properties of organic or inorganic substances. In fluorescence microscopy, a sample is illuminated with light of a wavelength which excites fluorescence in the sample. The fluoresced light, which is usually at a longer wavelength than the illumination, is then imaged through a microscope objective. Two filters may be used in this technique; an illumination (or excitation) filter which ensures the illumination is near monochromatic and at the correct wavelength, and a second emission (or barrier) filter which ensures none of the excitation light source reaches the detector. Alternatively, these functions may both be accomplished by a single dichroic filter. The “fluorescence microscope” comprises any microscope that uses fluorescence to generate an image, whether it is a more simple set up like an epifluorescence microscope, or a more complicated design such as a confocal microscope, which uses optical sectioning to get better resolution of the fluorescent image.

In some embodiments, confocal microscopy is used for detection and imaging of the detection probe. Confocal microscopy uses point illumination and a pinhole in an optically conjugate plane in front of the detector to eliminate out-of-focus signal. As only light produced by fluorescence very close to the focal plane can be detected, the image's optical resolution, particularly in the sample depth direction, is much better than that of wide-field microscopes. However, as much of the light from sample fluorescence is blocked at the pinhole, this increased resolution is at the cost of decreased signal intensity—so long exposures are often required. As only one point in the sample is illuminated at a time, 2D or 3D imaging requires scanning over a regular raster (e.g., a rectangular pattern of parallel scanning lines) in the specimen. The achievable thickness of the focal plane is defined mostly by the wavelength of the used light divided by the numerical aperture of the objective lens, but also by the optical properties of the specimen. The thin optical sectioning possible makes these types of microscopes particularly good at 3D imaging and surface profiling of samples. CLARITY™-optimized light sheet microscopy (COLM) provides an alternative microscopy for fast 3D imaging of large clarified samples. COLM interrogates large immunostained tissues, permits increased speed of acquisition and results in a higher quality of generated data.

Other types of microscopy that can be employed comprise bright field microscopy, oblique illumination microscopy, dark field microscopy, phase contrast, differential interference contrast (DIC) microscopy, interference reflection microscopy (also known as reflected interference contrast, or RIC), single plane illumination microscopy (SPIM), super-resolution microscopy, laser microscopy, electron microscopy (EM), Transmission electron microscopy (TEM), Scanning electron microscopy (SEM), reflection electron microscopy (REM), Scanning transmission electron microscopy (STEM) and low-voltage electron microscopy (LVEM), scanning probe microscopy (SPM), atomic force microscopy (ATM), ballistic electron emission microscopy (BEEM), chemical force microscopy (CFM), conductive atomic force microscopy (C-AFM), electrochemical scanning tunneling microscope (ECSTM), electrostatic force microscopy (EFM), fluidic force microscope (FluidFM), force modulation microscopy (FMM), feature-oriented scanning probe microscopy (FOSPM), kelvin probe force microscopy (KPFM), magnetic force microscopy (MFM), magnetic resonance force microscopy (MRFM), near-field scanning optical microscopy (NSOM) (or SNOM, scanning near-field optical microscopy, SNOM, Piezoresponse Force Microscopy (PFM), PSTM, photon scanning tunneling microscopy (PSTM), PTMS, photothermal microspectroscopy/microscopy (PTMS), SCM, scanning capacitance microscopy (SCM), SECM, scanning electrochemical microscopy (SECM), SGM, scanning gate microscopy (SGM), SHPM, scanning Hall probe microscopy (SHPM), SICM, scanning ion-conductance microscopy (SICM), SPSM spin polarized scanning tunneling microscopy (SPSM), SSRM, scanning spreading resistance microscopy (SSRM), SThM, scanning thermal microscopy (SThM), STM, scanning tunneling microscopy (STM), STP, scanning tunneling potentiometry (STP), SVM, scanning voltage microscopy (SVM), and synchrotron x-ray scanning tunneling microscopy (SXSTM), and intact tissue expansion microscopy (exM).

B. Substrates for In Situ Assay Modules

In some embodiments, a biological sample is provided on a first substrate for one or more in situ assay modules of the integrated assay disclosed herein. In some embodiments, the biological sample on the first substrate is contacted with one or more nucleic acid probes for one or more in situ assay modules. The one or more nucleic acid probes may directly or indirectly hybridize to a first target nucleic acid or a complement or an amplification product thereof in the biological sample. In some embodiments, the first substrate comprises a plurality of capture agents immobilized thereon, and the capture agents are capable of directly or indirectly capture a second target nucleic acid or a complement thereof or an amplification product thereof.

A wide variety of different substrates can be used for the in situ assay module, as long as the substrate is compatible with the sample and sample processing, the in situ reagents and reactions, and in situ signal detection (e.g., optical imaging such as fluorescence microscopy). A substrate can be any suitable support material and is generally transparent. For example, a glass slide such as a cover slip may be used. The first substrate can include, but are not limited to, glass, modified and/or functionalized glass, hydrogels, films, membranes, plastics, nylon, ceramics, resins, Zeonor, silica or silica-based materials including silicon and modified silicon, carbon, metals, inorganic glasses, optical fiber bundles, and polymers, such as polystyrene, cyclic olefin copolymers (COCs), cyclic olefin polymers (COPs), polypropylene, polyethylene and polycarbonate. The first substrate can also correspond to a flow cell.

In some embodiments, the first substrate is between about 0.01 mm and about 5 mm, e.g., between about 0.05 mm and about 3 mm, between about 0.1 mm and about 2.5 mm, between about 0.2 mm and about 2 mm, between about 0.5 mm and about 1.5 mm, or about 1 mm in thickness. In some embodiments, the first substrate is or is about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, or 2.0 mm in thickness, or of a thickness in between any of the aforementioned values.

### VIII. Compositions and Kits

Also provided herein are kits, for example comprising one or more oligonucleotides disclosed herein, and reagents for performing the methods provided herein, for example reagents required for one or more steps comprising hybridization, ligation, amplification, detection, sequencing, array preparation, analyte capture, and/or sample preparation as described herein. In some embodiments, the kit comprises one or more substrates (e.g., a first substrate and/or a second substrate). In some examples, a substrate may comprise a plurality of capture agents (e.g., capture probes) directly or indirectly immobilized thereon. In some embodiments, the kit further comprises a target nucleic acid. In some embodiments, any or all of the polynucleotides are DNA molecules. In some embodiments, the target nucleic acid is a messenger RNA molecule.

The various components of the kit may be present in separate containers or certain compatible components may be pre-combined into a single container. In some embodiments, the kits further contain instructions for using the components of the kit to practice the provided methods.

In some embodiments, the kits can contain reagents and/or consumables required for performing one or more steps of the provided methods. In some embodiments, the kits contain reagents for fixing, embedding, and/or permeabilizing the biological sample. In some embodiments, the kits contain reagents, such as enzymes and buffers for ligation and/or amplification, such as ligases and/or polymerases. In some aspects, the kit can also comprise any of the reagents described herein, e.g., wash buffer and ligation buffer. In some embodiments, the kits contain reagents for detection and/or sequencing, such as barcode detection probes or detectable labels. In some embodiments, the kits optionally contain other components, for example nucleic acid primers, enzymes and reagents, buffers, nucleotides, modified nucleotides, reagents for additional assays.

### IX. Terminology

Specific terminology is used throughout this disclosure to explain various aspects of the apparatus, systems, methods, and compositions that are described.

Having described some illustrative embodiments of the present disclosure, it should be apparent to those skilled in the art that the foregoing is merely illustrative and not limiting, having been presented by way of example only. Numerous modifications and other illustrative embodiments are within the scope of one of ordinary skill in the art and are contemplated as falling within the scope of the present disclosure. In particular, although many of the examples presented herein involve specific combinations of method acts or system elements, it should be understood that those acts and those elements may be combined in other ways to accomplish the same objectives.

As used herein, the singular forms “a,” “an,” and “the” include plural referents unless the context clearly dictates otherwise. For example, “a” or “an” means “at least one” or “one or more.”

The term “about” as used herein refers to the usual error range for the respective value readily known to the skilled person in this technical field. Reference to “about” a value or parameter herein includes (and describes) embodiments that are directed to that value or parameter per se.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range.

Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements. Similarly, use of a), b), etc., or i), ii), etc. does not by itself connote any priority, precedence, or order of steps in the claims. Similarly, the use of these terms in the specification does not by itself connote any required priority, precedence, or order.

(i) Barcode

A “barcode” is a label, or identifier, that conveys or is capable of conveying information (e.g., information about an analyte in a sample, a bead, and/or a capture probe). A barcode can be part of an analyte, or independent of an analyte. A barcode can be attached to an analyte. A particular barcode can be unique relative to other barcodes.

Barcodes can have a variety of different formats. For example, barcodes can include polynucleotide barcodes, random nucleic acid and/or amino acid sequences, and synthetic nucleic acid and/or amino acid sequences. A barcode can be attached to an analyte or to another moiety or structure in a reversible or irreversible manner. A barcode can be added to, for example, a fragment of a deoxyribonucleic acid (DNA) or ribonucleic acid (RNA) sample before or during sequencing of the sample. Barcodes can allow for identification and/or quantification of individual sequencing-reads (e.g., a barcode can be or can include a unique molecular identifier or “UMI”).

Barcodes can spatially-resolve molecular components found in biological samples, for example, at single-cell resolution (e.g., a barcode can be or can include a “spatial barcode”). In some embodiments, a barcode includes both a UMI and a spatial barcode. In some embodiments, a barcode includes two or more sub-barcodes that together function as a single barcode. For example, a polynucleotide barcode can include two or more polynucleotide sequences (e.g., sub-barcodes) that are separated by one or more non-barcode sequences.

(ii) Nucleic Acid and Nucleotide

The terms “nucleic acid” and “nucleotide” are intended to be consistent with their use in the art and to include naturally-occurring species or functional analogs thereof. Particularly useful functional analogs of nucleic acids are capable of hybridizing to a nucleic acid in a sequence-specific fashion (e.g., capable of hybridizing to two nucleic acids such that ligation can occur between the two hybridized nucleic acids) or are capable of being used as a template for replication of a particular nucleotide sequence. Naturally-occurring nucleic acids generally have a backbone containing phosphodiester bonds. An analog structure can have an alternate backbone linkage including any of a variety of suitable linkages. Naturally-occurring nucleic acids generally have a deoxyribose sugar (e.g., found in deoxyribonucleic acid (DNA)) or a ribose sugar (e.g. found in ribonucleic acid (RNA)).

A nucleic acid can contain nucleotides having any of a variety of analogs of suitable sugar moieties. A nucleic acid can include native or non-native nucleotides. In this regard, a native deoxyribonucleic acid can have one or more bases selected from the group consisting of adenine (A), thymine (T), cytosine (C), or guanine (G), and a ribonucleic acid can have one or more bases selected from the group consisting of uracil (U), adenine (A), cytosine (C), or guanine (G). Any suitable useful non-native bases can be included in a nucleic acid or nucleotide.

(iii) Probe and Target

A “probe” or a “target,” when used in reference to a nucleic acid or sequence of a nucleic acids, is intended as a semantic identifier for the nucleic acid or sequence in the context of a method or composition, and does not limit the structure or function of the nucleic acid or sequence beyond what is expressly indicated.

(iv) Oligonucleotide and Polynucleotide

The terms “oligonucleotide” and “polynucleotide” are used interchangeably to refer to a single-stranded multimer of nucleotides from about 2 to about 500 nucleotides in length. Oligonucleotides can be synthetic, made enzymatically (e.g., via polymerization), or using a “split-pool” method. Oligonucleotides can include ribonucleotide monomers (e.g., oligoribonucleotides) and/or deoxyribonucleotide monomers (e.g., oligodeoxyribonucleotides). In some examples, oligonucleotides can include a combination of both deoxyribonucleotide monomers and ribonucleotide monomers in the oligonucleotide (e.g., random or ordered combination of deoxyribonucleotide monomers and ribonucleotide monomers). An oligonucleotide can be 4 to 10, 10 to 20, 21 to 30, 31 to 40, 41 to 50, 51 to 60, 61 to 70, 71 to 80, 80 to 100, 100 to 150, 150 to 200, 200 to 250, 250 to 300, 300 to 350, 350 to 400, or 400-500 nucleotides in length, for example. Oligonucleotides can include one or more functional moieties that are attached (e.g., covalently or non-covalently) to the multimer structure. For example, an oligonucleotide can include one or more detectable labels (e.g., a radioisotope or fluorophore).

(v) Hybridizing, Hybridize, Annealing, and Anneal

The terms “hybridizing,” “hybridize,” “annealing,” and “anneal” are used interchangeably in this disclosure, and refer to the pairing of substantially complementary or complementary nucleic acid sequences within two different molecules. Pairing can be achieved by any process in which a nucleic acid sequence joins with a substantially or fully complementary sequence through base pairing to form a hybridization complex. For purposes of hybridization, two nucleic acid sequences are “substantially complementary” if at least 60% (e.g., at least 70%, at least 80%, or at least 90%) of their individual bases are complementary to one another.

(vi) Primer

A “primer” is a single-stranded nucleic acid sequence having a 3′ end that can be used as a substrate for a nucleic acid polymerase in a nucleic acid extension reaction. RNA primers are formed of RNA nucleotides, and are used in RNA synthesis, while DNA primers are formed of DNA nucleotides and used in DNA synthesis. Primers can also include both RNA nucleotides and DNA nucleotides (e.g., in a random or designed pattern). Primers can also include other natural or synthetic nucleotides described herein that can have additional functionality. In some examples, DNA primers can be used to prime RNA synthesis and vice versa (e.g., RNA primers can be used to prime DNA synthesis). Primers can vary in length. For example, primers can be about 6 bases to about 120 bases. For example, primers can include up to about 25 bases. A primer, may in some cases, refer to a primer binding sequence.

(vii) Primer Extension

Two nucleic acid sequences (e.g., a region from each of two distinct capture probes) can become linked (e.g., hybridized) by an overlap of their respective terminal complementary nucleic acid sequences (e.g., 3′ termini). Such linking can be followed by nucleic acid extension (e.g., an enzymatic extension) of one, or both termini using the other nucleic acid sequence as a template for extension. Enzymatic extension can be performed by an enzyme including, but not limited to, a polymerase and/or a reverse transcriptase.

(viii) Nucleic Acid Extension

A “nucleic acid extension” generally involves incorporation of one or more nucleic acids (e.g., A, G, C, T, U, nucleotide analogs, or derivatives thereof) into a molecule (such as, but not limited to, a nucleic acid sequence) in a template-dependent manner, such that consecutive nucleic acids are incorporated by an enzyme (such as a polymerase or reverse transcriptase), thereby generating a newly synthesized nucleic acid molecule. For example, a primer that hybridizes to a complementary nucleic acid sequence can be used to synthesize a new nucleic acid molecule by using the complementary nucleic acid sequence as a template for nucleic acid synthesis. Similarly, a 3′ polyadenylated tail of an mRNA transcript that hybridizes to a poly (dT) sequence (e.g., capture domain) can be used as a template for single-strand synthesis of a corresponding cDNA molecule.

(ix) PCR Amplification

A “PCR amplification” refers to the use of a polymerase chain reaction (PCR) to generate copies of genetic material, including DNA and RNA sequences. Suitable reagents and conditions for implementing PCR are described, for example, in U.S. Pat. Nos. 4,683,202, 4,683,195, 4,800,159, 4,965,188, and 5,512,462, the entire contents of each of which are incorporated herein by reference. In a typical PCR amplification, the reaction mixture includes the genetic material to be amplified, an enzyme, one or more primers that are employed in a primer extension reaction, and reagents for the reaction. The oligonucleotide primers are of sufficient length to provide for hybridization to complementary genetic material under annealing conditions. The length of the primers generally depends on the length of the amplification domains, but will typically be at least 4 bases, at least 5 bases, at least 6 bases, at least 8 bases, at least 9 bases, at least 10 base pairs (bp), at least 11 bp, at least 12 bp, at least 13 bp, at least 14 bp, at least 15 bp, at least 16 bp, at least 17 bp, at least 18 bp, at least 19 bp, at least 20 bp, at least 25 bp, at least 30 bp, at least 35 bp, and can be as long as 40 bp or longer, where the length of the primers will generally range from 18 to 50 bp. The genetic material can be contacted with a single primer or a set of two primers (forward and reverse primers), depending upon whether primer extension, linear or exponential amplification of the genetic material is desired.

In some embodiments, the PCR amplification process uses a DNA polymerase enzyme. The DNA polymerase activity can be provided by one or more distinct DNA polymerase enzymes. In certain embodiments, the DNA polymerase enzyme is from a bacterium, e.g., the DNA polymerase enzyme is a bacterial DNA polymerase enzyme. For instance, the DNA polymerase can be from a bacterium of the genus Escherichia, Bacillus, Thermophilus, or Pyrococcus.

Suitable examples of DNA polymerases that can be used include, but are not limited to: E. coli DNA polymerase I, Bsu DNA polymerase, Bst DNA polymerase, Taq DNA polymerase, VENT™ DNA polymerase, DEEPVENT™ DNA polymerase, LongAmp® Taq DNA polymerase, LongAmp® Hot Start Taq DNA polymerase, Crimson LongAmp® Taq DNA polymerase, Crimson Taq DNA polymerase, OneTaq® DNA polymerase, OneTaq® Quick-Load® DNA polymerase, Hemo KlenTaq® DNA polymerase, REDTaq® DNA polymerase, Phusion® DNA polymerase, Phusion® High-Fidelity DNA polymerase, Platinum Pfx DNA polymerase, AccuPrime Pfx DNA polymerase, Phi29 DNA polymerase, Klenow fragment, Pwo DNA polymerase, Pfu DNA polymerase, T4 DNA polymerase and T7 DNA polymerase enzymes.

The term “DNA polymerase” includes not only naturally-occurring enzymes but also all modified derivatives thereof, including also derivatives of naturally-occurring DNA polymerase enzymes. For instance, in some embodiments, the DNA polymerase can have been modified to remove 5′-3′ exonuclease activity. Sequence-modified derivatives or mutants of DNA polymerase enzymes that can be used include, but are not limited to, mutants that retain at least some of the functional, e.g. DNA polymerase activity of the wild-type sequence. Mutations can affect the activity profile of the enzymes, e.g. enhance or reduce the rate of polymerization, under different reaction conditions, e.g. temperature, template concentration, primer concentration, etc. Mutations or sequence-modifications can also affect the exonuclease activity and/or thermostability of the enzyme.

In some embodiments, PCR amplification can include reactions such as, but not limited to, a strand-displacement amplification reaction, a rolling circle amplification reaction, a ligase chain reaction, a transcription-mediated amplification reaction, an isothermal amplification reaction, and/or a loop-mediated amplification reaction.

In some embodiments, PCR amplification uses a single primer that is complementary to the 3′ tag of target DNA fragments. In some embodiments, PCR amplification uses a first and a second primer, where at least a 3′ end portion of the first primer is complementary to at least a portion of the 3′ tag of the target nucleic acid fragments, and where at least a 3′ end portion of the second primer exhibits the sequence of at least a portion of the 5′ tag of the target nucleic acid fragments. In some embodiments, a 5′ end portion of the first primer is non-complementary to the 3′ tag of the target nucleic acid fragments, and a 5′ end portion of the second primer does not exhibit the sequence of at least a portion of the 5′ tag of the target nucleic acid fragments. In some embodiments, the first primer includes a first universal sequence and/or the second primer includes a second universal sequence.

In some embodiments (e.g., when the PCR amplification amplifies captured DNA), the PCR amplification products can be ligated to additional sequences using a DNA ligase enzyme. The DNA ligase activity can be provided by one or more distinct DNA ligase enzymes. In some embodiments, the DNA ligase enzyme is from a bacterium, e.g., the DNA ligase enzyme is a bacterial DNA ligase enzyme. In some embodiments, the DNA ligase enzyme is from a virus (e.g., a bacteriophage). For instance, the DNA ligase can be T4 DNA ligase. Other enzymes appropriate for the ligation step include, but are not limited to, Tth DNA ligase, Taq DNA ligase, Thermococcus sp. (strain 9oN) DNA ligase (9oN™ DNA ligase, available from New England Biolabs, Ipswich, Mass.), and Ampligase™ (available from Epicentre Biotechnologies, Madison, Wis.). Derivatives, e.g. sequence-modified derivatives, and/or mutants thereof, can also be used.

In some embodiments, genetic material is amplified by reverse transcription polymerase chain reaction (RT-PCR). The desired reverse transcriptase activity can be provided by one or more distinct reverse transcriptase enzymes, suitable examples of which include, but are not limited to: M-MLV, MuLV, AMV, HIV, ArrayScript™, MultiScribe™ ThermoScript™, and SuperScript® I, II, III, and IV enzymes. “Reverse transcriptase” includes not only naturally occurring enzymes, but all such modified derivatives thereof, including also derivatives of naturally-occurring reverse transcriptase enzymes.

In addition, reverse transcription can be performed using sequence-modified derivatives or mutants of M-MLV, MuLV, AMV, and HIV reverse transcriptase enzymes, including mutants that retain at least some of the functional, e.g. reverse transcriptase, activity of the wild-type sequence. The reverse transcriptase enzyme can be provided as part of a composition that includes other components, e.g. stabilizing components that enhance or improve the activity of the reverse transcriptase enzyme, such as RNase inhibitor(s), inhibitors of DNA-dependent DNA synthesis, e.g. actinomycin D. Many sequence-modified derivative or mutants of reverse transcriptase enzymes, e.g. M-MLV, and compositions including unmodified and modified enzymes are commercially available, e.g. ArrayScript™, MultiScribe™ ThermoScript™, and SuperScript® I, II, III, and IV enzymes.

Certain reverse transcriptase enzymes (e.g. Avian Myeloblastosis Virus (AMV) Reverse Transcriptase and Moloney Murine Leukemia Virus (M-MuLV, MMLV) Reverse Transcriptase) can synthesize a complementary DNA strand using both RNA (cDNA synthesis) and single-stranded DNA (ssDNA) as a template. Thus, in some embodiments, the reverse transcription reaction can use an enzyme (reverse transcriptase) that is capable of using both RNA and ssDNA as the template for an extension reaction, e.g. an AMV or MMLV reverse transcriptase.

In some embodiments, the quantification of RNA and/or DNA is carried out by real-time PCR (also known as quantitative PCR or qPCR), using any suitable techniques, such as but not limited to “TAQMAN™” or “SYBR®”, or on capillaries (“LightCycler® Capillaries”). In some embodiments, the quantification of genetic material is determined by optical absorbance and with real-time PCR. In some embodiments, the quantification of genetic material is determined by digital PCR. In some embodiments, the genes analyzed can be compared to a reference nucleic acid extract (DNA and RNA) corresponding to the expression (mRNA) and quantity (DNA) in order to compare expression levels of the target nucleic acids.

(x) Antibody

An “antibody” is a polypeptide molecule that recognizes and binds to a complementary target antigen. Antibodies typically have a molecular structure shape that resembles a Y shape. Naturally-occurring antibodies, referred to as immunoglobulins, belong to one of the immunoglobulin classes IgG, IgM, IgA, IgD, and IgE. Antibodies can also be produced synthetically. For example, recombinant antibodies, which are monoclonal antibodies, can be synthesized using synthetic genes by recovering the antibody genes from source cells, amplifying into an appropriate vector, and introducing the vector into a host to cause the host to express the recombinant antibody. In general, recombinant antibodies can be cloned from any species of antibody-producing animal using suitable oligonucleotide primers and/or hybridization probes. Recombinant techniques can be used to generate antibodies and antibody fragments, including non-endogenous species.

Synthetic antibodies can be derived from non-immunoglobulin sources. For example, antibodies can be generated from nucleic acids (e.g., aptamers), and from non-immunoglobulin protein scaffolds (such as peptide aptamers) into which hypervariable loops are inserted to form antigen binding sites. Synthetic antibodies based on nucleic acids or peptide structures can be smaller than immunoglobulin-derived antibodies, leading to greater tissue penetration.

Antibodies can also include affimer proteins, which are affinity reagents that typically have a molecular weight of about 12-14 kDa. Affimer proteins generally bind to a target (e.g., a target protein) with both high affinity and specificity. Examples of such targets include, but are not limited to, ubiquitin chains, immunoglobulins, and C-reactive protein. In some embodiments, affimer proteins are derived from cysteine protease inhibitors, and include peptide loops and a variable N-terminal sequence that provides the binding site.

Antibodies can also refer to an “epitope binding fragment” or “antibody fragment,” which as used herein, generally refers to a portion of a complete antibody capable of binding the same epitope as the complete antibody, albeit not necessarily to the same extent. Although multiple types of epitope binding fragments are possible, an epitope binding fragment typically comprises at least one pair of heavy and light chain variable regions (VH and VL, respectively) held together (e.g., by disulfide bonds) to preserve the antigen binding site, and does not contain all or a portion of the Fc region. Epitope binding fragments of an antibody can be obtained from a given antibody by any suitable technique (e.g., recombinant DNA technology or enzymatic or chemical cleavage of a complete antibody), and typically can be screened for specificity in the same manner in which complete antibodies are screened. In some embodiments, an epitope binding fragment comprises an F(ab′)₂ fragment, Fab′ fragment, Fab fragment, Fd fragment, or Fv fragment. In some embodiments, the term “antibody” includes antibody-derived polypeptides, such as single chain variable fragments (scFv), diabodies or other multimeric scFvs, heavy chain antibodies, single domain antibodies, or other polypeptides comprising a sufficient portion of an antibody (e.g., one or more complementarity determining regions (CDRs)) to confer specific antigen binding ability to the polypeptide.

(xi) Label, Detectable Label, and Optical Label

The terms “detectable label,” “optical label,” and “label” are used interchangeably herein to refer to a directly or indirectly detectable moiety that is associated with (e.g., conjugated to) a molecule to be detected, e.g., a probe for in situ assay, a capture probe or analyte. The detectable label can be directly detectable by itself (e.g., radioisotope labels or fluorescent labels) or, in the case of an enzymatic label, can be indirectly detectable, e.g., by catalyzing chemical alterations of a substrate compound or composition, which substrate compound or composition is directly detectable. Detectable labels can be suitable for small scale detection and/or suitable for high-throughput screening. As such, suitable detectable labels include, but are not limited to, radioisotopes, fluorophores, chemiluminescent compounds, bioluminescent compounds, and dyes.

The detectable label can be qualitatively detected (e.g., optically or spectrally), or it can be quantified. Qualitative detection generally includes a detection method in which the existence or presence of the detectable label is confirmed, whereas quantifiable detection generally includes a detection method having a quantifiable (e.g., numerically reportable) value such as an intensity, duration, polarization, and/or other properties. In some embodiments, the detectable label is bound to a feature or to a capture probe associated with a feature. For example, detectably labeled features can include a fluorescent, a colorimetric, or a chemiluminescent label attached to a bead (see, for example, Rajeswari et al., J. Microbiol Methods 139:22-28, 2017, and Forcucci et al., J. Biomed Opt. 10:105010, 2015, the entire contents of each of which are incorporated herein by reference).

In some embodiments, a plurality of detectable labels can be attached to a feature, capture probe, or composition to be detected. For example, detectable labels can be incorporated during nucleic acid polymerization or amplification (e.g., Cy5®-labeled nucleotides, such as Cy5®-dCTP). Any suitable detectable label can be used. In some embodiments, the detectable label is a fluorophore. For example, the fluorophore can be from a group that includes: 7-AAD (7-Aminoactinomycin D), Acridine Orange (+DNA), Acridine Orange (+RNA), Alexa Fluor® 350, Alexa Fluor® 430, Alexa Fluor® 488, Alexa Fluor® 532, Alexa Fluor® 546, Alexa Fluor® 555, Alexa Fluor® 568, Alexa Fluor® 594, Alexa Fluor® 633, Alexa Fluor® 647, Alexa Fluor® 660, Alexa Fluor® 680, Alexa Fluor® 700, Alexa Fluor® 750, Allophycocyanin (APC), AMCA/AMCA-X, 7-Aminoactinomycin D (7-AAD), 7-Amino-4-methylcoumarin, 6-Aminoquinoline, Aniline Blue, ANS, APC-Cy7, ATTO-TAG™ CBQCA, ATTO-TAG™ FQ, Auramine O-Feulgen, BCECF (high pH), BFP (Blue Fluorescent Protein), BFP/GFP FRET, BOBO™-1/BO-PRO™-1, BOBO™-3/BO-PRO™-3, BODIPY® FL, BODIPY® TMR, BODIPY® TR-X, BODIPY® 530/550, BODIPY® 558/568, BODIPY® 564/570, BODIPY® 581/591, BODIPY® 630/650-X, BODIPY® 650-665-X, BTC, Calcein, Calcein Blue, Calcium Crimson™, Calcium Green-1™, Calcium Orange™, Calcofluor® White, 5-Carboxyfluoroscein (5-FAM), 5-Carboxynaphthofluoroscein, 6-Carboxyrhodamine 6G, 5-Carboxytetramethylrhodamine (5-TAMRA), Carboxy-X-rhodamine (5-ROX), Cascade Blue®, Cascade Yellow™, CCF2 (GeneBLAzer™), CFP (Cyan Fluorescent Protein), CFP/YFP FRET, Chromomycin A3, Cl-NERF (low pH), CPM, 6-CR 6G, CTC Formazan, Cy2®, Cy3®, Cy3.5®, Cy5®, Cy5.5®, Cy7®, Cychrome (PE-Cy5), Dansylamine, Dansyl cadaverine, Dansylchloride, DAPI, Dapoxyl, DCFH, DHR, DiA (4-Di-16-ASP), DiD (DilC18(5)), DIDS, Dil (DilC18(3)), DiO (DiOC18(3)), DiR (DilC18(7)), Di-4 ANEPPS, Di-8 ANEPPS, DM-NERF (4.5-6.5 pH), DsRed (Red Fluorescent Protein), EBFP, ECFP, EGFP, ELF®-97 alcohol, Eosin, Erythrosin, Ethidium bromide, Ethidium homodimer-1 (EthD-1), Europium (III) Chloride, 5-FAM (5-Carboxyfluorescein), Fast Blue, Fluorescein-dT phosphoramidite, FITC, Fluo-3, Fluo-4, FluorX®, Fluoro-Gold™ (high pH), Fluoro-Gold™ (low pH), Fluoro-Jade, FM® 1-43, Fura-2 (high calcium), Fura-2/BCECF, Fura Red™ (high calcium), Fura Red™/Fluo-3, GeneBLAzer™ (CCF2), GFP Red Shifted (rsGFP), GFP Wild Type, GFP/BFP FRET, GFP/DsRed FRET, Hoechst 33342 & 33258, 7-Hydroxy-4-methylcoumarin (pH 9), 1,5 IAEDANS, Indo-1 (high calcium), Indo-1 (low calcium), Indodicarbocyanine, Indotricarbocyanine, JC-1, 6-JOE, JOJO™1/JO-PRO™-1, LDS 751 (+DNA), LDS 751 (+RNA), LOLO™-1/LO-PRO™-1, Lucifer Yellow, LysoSensor™ Blue (pH 5), LysoSensor™ Green (pH 5), LysoSensor™ Yellow/Blue (pH 4.2), LysoTracker® Green, LysoTracker® Red, LysoTracker® Yellow, Mag-Fura-2, Mag-Indo-1, Magnesium Green™, Marina Blue®, 4-Methylumbelliferone, Mithramycin, Mito Tracker® Green, Mito Tracker® Orange, Mito Tracker® Red, NBD (amine), Nile Red, Oregon Green® 488, Oregon Green® 500, Oregon Green® 514, Pacific Blue, PBF1, PE (R-phycoerythrin), PE-Cy5, PE-Cy7, PE-Texas Red, PerCP (Peridinin chlorphyll protein), PerCP-Cy5.5 (TruRed), PharRed (APC-Cy7), C-phycocyanin, R-phycocyanin, R-phycoerythrin (PE), PI (Propidium Iodide), PKH26, PKH67, POPO™1/PO-PRO™-1, POPO™3/PO-PRO™-3, Propidium Iodide (PI), PyMPO, Pyrene, Pyronin Y, Quantam Red (PE-Cy5), Quinacrine Mustard, R670 (PE-Cy5), Red 613 (PE-Texas Red), Red Fluorescent Protein (DsRed), Resorufin, RH 414, Rhod-2, Rhodamine B, Rhodamine Green™, Rhodamine Red™, Rhodamine Phalloidin, Rhodamine 110, Rhodamine 123, 5-ROX (carboxy-X-rhodamine), S65A, S65C, S65L, S65T, SBFI, SITS, SNAFL®-1 (high pH), SNAFL®-2, SNARF®-1 (high pH), SNARF®-1 (low pH), Sodium Green™, SpectrumAqua®, SpectrumGreen® #1, SpectrumGreen® #2, SpectrumOrange®, SpectrumRed®, SYTO® 11, SYTO® 13, SYTO® 17, SYTO® 45, SYTOX® Blue, SYTOX® Green, SYTOX® Orange, 5-TAMRA (5-Carboxytetramethylrhodamine), Tetramethylrhodamine (TRITC), Texas Red®/Texas Red®-X, Texas Red®-X (NHS Ester), Thiadicarbocyanine, Thiazole Orange, TOTO®-1/TO-PRO®-1, TOTO®-3/TO-PRO®-3, TO-PRO®-5, Tri-color (PE-Cy5), TRITC (Tetramethylrhodamine), TruRed (PerCP-Cy5.5), WW 781, X-Rhodamine (XRITC), Y66F, Y66H, Y66W, YFP (Yellow Fluorescent Protein), YOYO®-1/YO-PRO®-1, YOYO®-3/YO-PRO®-3, 6-FAM (Fluorescein), 6-FAM (NHS Ester), 6-FAM (Azide), HEX, TAMRA (NHS Ester), Yakima Yellow, MAX, TET, TEX615, ATTO 488, ATTO 532, ATTO 550, ATTO 565, ATTO Rhol01, ATTO 590, ATTO 633, ATTO 647N, TYE 563, TYE 665, TYE 705, 5′ IRDye® 700, 5′ IRDye® 800, 5′ IRDye® 800CW (NHS Ester), WellRED D4 Dye, WellRED D3 Dye, WellRED D2 Dye, Lightcycler® 640 (NHS Ester), and Dy 750 (NHS Ester).

As mentioned above, in some embodiments, a detectable label is or includes a luminescent or chemiluminescent moiety. Common luminescent/chemiluminescent moieties include, but are not limited to, peroxidases such as horseradish peroxidase (HRP), soybean peroxidase (SP), alkaline phosphatase, and luciferase. These protein moieties can catalyze chemiluminescent reactions given the appropriate substrates (e.g., an oxidizing reagent plus a chemiluminescent compound. A number of compound families are known to provide chemiluminescence under a variety of conditions. Non-limiting examples of chemiluminescent compound families include 2,3-dihydro-1,4-phthalazinedione luminol, 5-amino-6,7,8-trimethoxy- and the dimethylamino[ca]benz analog. These compounds can luminesce in the presence of alkaline hydrogen peroxide or calcium hypochlorite and base. Other examples of chemiluminescent compound families include, e.g., 2,4,5-triphenylimidazoles, para-dimethylamino and—methoxy substituents, oxalates such as oxalyl active esters, p-nitrophenyl, N-alkyl acridinum esters, luciferins, lucigenins, or acridinium esters. In some embodiments, a detectable label is or includes a metal-based or mass-based label. For example, small cluster metal ions, metals, or semiconductors may act as a mass code. In some examples, the metals can be selected from Groups 3-15 of the periodic table, e.g., Y, La, Ag, Au, Pt, Ni, Pd, Rh, Ir, Co, Cu, Bi, or a combination thereof.

### EXAMPLE

The following example is included for illustrative purposes only and is not intended to limit the scope of the present disclosure.

### Example 1: Targeting and Capture of Nearby mRNA by Monomers

FIG. 2 provides an exemplary method of this in situ assay. FIG. 2 shows the targeting of a specific analyte (e.g., a specific protein) by an analyte-binding moiety (e.g., an antibody) conjugated with a reporter oligonucleotide and a nucleic acid probe (e.g., a circularizable probe such as a padlock probe) hybridized to the oligonucleotide. The nucleic acid probe (e.g., a circularizable probe such as a padlock probe) comprises specific sequence motifs described including an adapter, barcodes, nucleic acid capture sequence, and a restriction cleavage site, as depicted in FIG. 1. In the exemplary method, the analyte-binding moiety (e.g., an antibody) binds to the target analyte on the biological sample (e.g., a tissue section), unbound antibodies are washed off of the biological sample (e.g., a tissue section), and rolling circle amplification of the probe which was hybridized to the reporter oligonucleotide begins.

In the exemplary method, the amplification of the nucleic acid probe (e.g., a padlock probe) generates a concatemer of monomer sequences, wherein each monomer is complementary to the sequence of the probe. The concatemer will be detected at a spatial location of the biological sample by reading the protein barcode of the probe in situ by way of sequencing and/or in situ hybridization. The protein barcode will be used identify the specific antibody to which the oligonucleotide and padlock probe was bound to while the spatial barcode is capable of conveying spatial information. The concatemer will be cleaved at the restriction sites producing monomers which are now free to diffuse into the surrounding tissue. Importantly, each monomer comprises a oligo dT capture sequence on the 3′ end. Upon diffusing, the oligo dT capture sequence of the monomer will target the polyA tail of nearby mRNAs.

Once captured, the monomer will hybridize to the nearby mRNA and reverse transcription will occur by using the complement in the monomers as a reverse transcription primer. The generated library of tagged cDNA can be altered by ligating or adding a second adapter sequence to the tagged cDNA. Additionally, the tagged cDNA can be amplified prior to being analyzed by direct sequencing or indirect sequencing. In this exemplary method, the portion of the tagged cDNA that is sequenced comprises at least the barcode sequence (or optionally the spatial barcode sequence) and a portion of the target nucleic acid or complement thereof.

### Example 2: Targeting and Capture of Circularized cDNA by Monomers

FIG. 3 provides an exemplary method of this in situ assay. FIG. 3 shows an RNA target reverse transcribed to generate a DNA molecule which is then circularized by way of a single-stranded DNA ligase that catalyzes intramolecular ligation. In this exemplary method, an analyte-binding moiety (e.g., an antibody) binds to the target analyte (e.g., a specific protein) wherein the analyte-binding moiety is conjugated to a reporter oligonucleotide hybridized to a nucleic acid probe (e.g., a circularizable probe such a padlock probe) and the probe is amplified by RCA to generate a concatemer of complementary monomers. The nucleic acid probe comprises specific sequence motifs described including an adapter, barcodes, nucleic acid capture sequence, and a restriction cleavage site, as depicted in FIG. 1.

The resulting concatemer will be detected at a spatial location of the biological sample (e.g., a tissue sample) by in situ sequencing and/or in situ hybridization. The concatemer will be cleaved into monomers at the restriction cleavage site by restriction endonucleases. The monomers produced by the cleavage of the concatemer comprise an oligo dA capture sequence on the 3′ end. The oligo dT sequence of the circularized cDNA can hybridize to the 3′ oligo dA sequence of a nearby cleaved RCP products (monomers). At this point, a library of tagged cDNA will be generated by priming the extension of the circularized cDNA with the complement in the monomer as an extension primer. The library of tagged cDNA will then be analyzed by sequencing.

The generated library of tagged cDNA can be altered by ligating or adding a second adapter sequence to the tagged cDNA. Additionally, the tagged cDNA can be amplified prior to being analyzed by direct sequencing or indirect sequencing. The amplified sequence will include the protein barcode sequence and/or the spatial barcode sequence. In this exemplary method, the protein barcode will be read in situ and be used to identify the specific antibody to which the oligonucleotide and padlock probe was bound to. The optional spatial barcode will be used to define the position of the single antibody in situ.

### Example 3: Targeting and Capture of Second Strand cDNA by Monomers

In this exemplary method, a target nucleic acid is generated in a biological sample, wherein the target nucleic acid comprises a second-strand cDNA of an mRNA molecule. This second-strand cDNA is generated by way of reverse transcription of the mRNA molecule followed by the use of a template-switch oligonucleotide (TSO).

The biological sample is contacted by the analyte-binding moiety (e.g., an antibody) of FIG. 1. As described in Example 1, the analyte-binding moiety binds to the target analyte (e.g., a specific protein) on the biological sample (e.g., a tissue section), unbound antibodies are washed off of the biological sample, and rolling circle amplification of the nucleic acid probe which was hybridized to the reporter oligonucleotide begins. The generated concatemer can be detected at a spatial location of the biological sample by in situ sequencing and/or in situ hybridization.

As described in Example 2, the resulting concatemer will be detected at a spatial location of the biological sample by in situ sequencing and/or in situ hybridization. The concatemer will be cleaved into monomers at the restriction cleavage site by restriction endonucleases. The monomers produced by the cleavage of the concatemer comprise an oligo dT capture sequence on the 3′ end and the monomers will hybridize to the target nucleic acid via the complement. At this point, a library of tagged cDNA will be generated by priming the extension of the circularized cDNA with the complement in the monomer as an extension primer. The library of tagged cDNA will then be analyzed by sequencing.

The generated library of tagged cDNA can be altered by ligating or adding a second adapter sequence to the tagged cDNA. Additionally, the tagged cDNA can be amplified prior to being analyzed by direct sequencing or indirect sequencing. The amplified sequence will include the protein barcode sequence and/or the spatial barcode sequence. In this exemplary method, the protein barcode will be read in situ and be used to identify the specific antibody to which the oligonucleotide and padlock probe was bound to. The optional spatial barcode will be used to define the position of the single antibody in situ.

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

1. A method of analyzing a biological sample, comprising:
(a) contacting a biological sample with:
a labeling agent comprising an analyte-binding moiety conjugated to a reporter oligonucleotide, and
a nucleic acid probe comprising a cassette comprising (1) a restriction site, (2) a nucleic acid capture sequence, and (3) a barcode sequence, wherein the nucleic acid probe hybridizes to the reporter oligonucleotide;
(b) amplifying the nucleic acid probe to generate a concatemer of monomer sequences, wherein each monomer sequence comprises sequences that are complementary to the cassette;
(c) generating a plurality of monomers by cleaving the concatemer at the restriction site,
wherein a monomer of the plurality of monomers comprises a complement of the nucleic acid capture sequence at the 3′ end; and
(d) allowing the monomer to hybridize to a target nucleic acid in the biological sample via the complement.

2. The method of claim 1, further comprising, before step (c), detecting the concatemer or a portion thereof at a spatial location of the biological sample.

3. The method of claim 1 or 2, wherein the nucleic acid capture sequence is or comprises a plurality of adenine (A) polynucleotides, a plurality of thymine (T) nucleotides, or a gene specific sequence.

4. The method of any of claims 1-3, wherein the cleaving the concatemer at the restriction site in step (c) comprises contacting the biological sample with a restriction endonuclease that recognizes the restriction site.

5. The method of any of claims 1-4, wherein the target nucleic acid comprises an RNA molecule present in the biological sample or a sequence thereof, a reverse transcription product thereof, a complement thereof, a hybridization product thereof, a ligation product thereof, an extension product thereof, a replication product thereof, and/or an amplification product thereof.

6. The method of any of claims 1-5, wherein the target nucleic acid comprises a messenger RNA (mRNA) molecules present at or near the a labeling agent in the biological sample or a reverse transcription product thereof.

7. The method of any of claims 1-6, wherein the target nucleic acid comprises an individual mRNA molecule transcribed from a particular gene, and the nucleic acid capture sequence is or comprises a gene specific sequence.

8. The method of any of claims 1-7, wherein the target nucleic acid comprises a reverse transcription product of an mRNA molecule.

9. The method of claim 8, further comprising, before step (d), generating a reverse transcription product of an mRNA molecule present in the biological sample.

10. The method of claim 8 or 9, wherein the reverse transcription product is a second-strand cDNA.

11. The method of any of claims 1-7, wherein the target nucleic acid comprises mRNA molecules that comprises a polyA tail.

12. The method of any of claims 8-11, wherein the nucleic acid capture sequence is or comprises a plurality of adenine (A) nucleotides.

13. The method of claim 11, wherein the plurality of adenine (A) nucleotides comprises at least 2, 3, 4, 5, 6, 7, 8, 9, 10, 15, 20, 25, 30, 35, 40, 45, 50, 60, 70, 80, 90, 100 or more adenine (A) nucleotides.

14. The method of any of claims 4-13, wherein the restriction endonuclease is selected from among DraI, MseI, TaqI-v2 and XbaI.

15. The method of any one of claims 11-14, further comprising generating a library of tagged complementary DNA (cDNA) by performing reverse transcription of the target nucleic acids using the complement in the monomer as a reverse transcription primer.

16. The method of any of claims 8 and 9, wherein the reverse transcription product is a circularized cDNA.

17. The method of any of claims 1-9 and 16, wherein the nucleic acid capture sequence is or comprises a plurality of thymine (T) nucleotides.

18. The method of claim 17, wherein the plurality of thymine (T) nucleotides comprises at least 2, 3, 4, 5, 6, 7, 8, 9, 10, 15, 20, 25, 30, 35, 40, 45, 50, 60, 70, 80, 90, 100 or more thymine (T) nucleotides.

19. The method of any of claims 4-9 and 16-18, wherein the restriction endonuclease is selected from among AcII, AgeI, BgIII, HindIII, SpeI, MluI, and PciI.

20. The method of any of claims 8-10 and 16-19, further comprising, after step (d), generating a library of tagged cDNA by priming the extension of the cDNA using the complement in the monomer as an extension primer.

21. The method of claim 15 or 20, further comprising, analyzing the library of tagged cDNA by sequencing the tagged cDNAs or a portion thereof.

22. A method of analyzing a biological sample, comprising:
(a) contacting a biological sample with:
a labeling agent comprising an analyte-binding moiety conjugated to a reporter oligonucleotide, and
a nucleic acid probe comprising a cassette comprising (1) a restriction site, (2) a plurality of adenine (A) nucleotides, (3) a barcode sequence comprising an analyte-binding moiety-specific barcode sequence and a spatial barcode sequence and (4) an adapter sequence, wherein the nucleic acid probe hybridizes to the reporter oligonucleotide;
(b) amplifying the nucleic acid probe to generate a concatemer of monomer sequences by rolling circle amplification (RCA), wherein each monomer sequence comprises sequences that are complementary to the cassette;
(c) detecting the concatemer or a portion thereof at a spatial location of the biological sample by in situ sequencing and/or in situ hybridization;
(d) generating a plurality of monomers by cleaving the concatemer at the restriction site,
wherein a monomer of the plurality of monomers comprises a complement of the nucleic acid capture sequence at the 3′ end that comprises an oligo dT sequence;
(e) allowing the monomer to hybridize to a target nucleic acid via the complement, wherein the target nucleic acid comprises an mRNA molecule present at or near the labeling agent in the biological sample;
(f) generating a library of tagged complementary DNA (cDNA) by performing reverse transcription of the target nucleic acids using the complement in the monomers as a reverse transcription primer; and
(g) analyzing the library of tagged cDNA by sequencing the tagged cDNAs or a portion thereof.

23. A method of analyzing a biological sample, comprising:
(a) generating, in a biological sample a target nucleic acid that comprises a circular cDNA that is complementary to an mRNA molecule in the biological sample, by reverse transcription of the mRNA molecule, and circularizing the cDNA;
(b) contacting the biological sample with:
a labeling agent comprising an analyte-binding moiety conjugated to a reporter oligonucleotide, and
a nucleic acid probe comprising a cassette comprising (1) a restriction site, (2) a plurality of thymine (T) nucleotides, (3) a barcode sequence comprising an analyte-binding moiety-specific barcode sequence and a spatial barcode sequence and (4) an adapter sequence, wherein the nucleic acid probe hybridizes to the reporter oligonucleotide;
(c) amplifying the nucleic acid probe to generate a concatemer of monomer sequences by rolling circle amplification (RCA), wherein each monomer sequence comprises sequences that are complementary to the cassette;
(d) detecting the concatemer or a portion thereof at a spatial location of the biological sample by in situ sequencing and/or in situ hybridization;
(e) generating a plurality of monomers by cleaving the concatemer at the restriction site,
wherein a monomer of the plurality of monomers comprises a complement of the nucleic acid capture sequence at the 3′ end that comprises an oligo dA sequence;
(f) allowing the monomer to hybridize to the target nucleic acid via the complement;
(g) generating a library of tagged cDNA by priming the extension of the circularized cDNA with the complement in the monomer; and
(h) analyzing the library of tagged cDNA by sequencing the tagged cDNAs or a portion thereof.

24. A method of analyzing a biological sample, comprising:
(a) generating, in a biological sample a target nucleic acid that comprises a second-strand cDNA of an mRNA molecule in the biological sample, by reverse transcription of the mRNA molecule and generating the second-strand cDNA using a template-switch oligonucleotide (TSO);
(b) contacting a biological sample with:
a labeling agent comprising an analyte-binding moiety conjugated to a reporter oligonucleotide, and
a nucleic acid probe comprising a cassette comprising (1) a restriction site, (2) a plurality of adenine (A) nucleotides, (3) a barcode sequence comprising an analyte-binding moiety-specific barcode sequence and a spatial barcode sequence and (4) an adapter sequence, wherein the nucleic acid probe hybridizes to the reporter oligonucleotide;
(c) amplifying the nucleic acid probe to generate a concatemer of monomer sequences by rolling circle amplification (RCA), wherein each monomer sequence comprises sequences that are complementary to the cassette;
(d) detecting the concatemer or a portion thereof at a spatial location of the biological sample by in situ sequencing and/or in situ hybridization;
(e) generating a plurality of monomers by cleaving the concatemer at the restriction site,
wherein a monomer of the plurality of monomers comprises a complement of the nucleic acid capture sequence at the 3′ end that comprises an oligo dT sequence;
(f) allowing the monomer to hybridize to the target nucleic acid via the complement;
(g) generating a library of tagged cDNA by priming the extension of the second strand cDNA with the complement in the monomer; and
(h) analyzing the library of tagged cDNA by sequencing the tagged cDNAs or a portion thereof.

25. The method of any of claims 1-24, wherein the barcode sequence comprises an analyte-binding moiety-specific barcode sequence.

26. The method of claim 25, wherein the analyte-binding moiety-specific barcode sequence uniquely identifies the analyte-binding moiety among the labeling agent comprising different analyte-binding moieties.

27. The method of any of claims 1-26, wherein the barcode sequence further comprises a spatial barcode sequence.

28. The method of claim 27, wherein the spatial barcode sequence uniquely identifies a single labeling agent molecule.

29. The method of claim 27 or 28, wherein the spatial barcode sequence corresponds to the position of the single labeling agent molecule in the biological sample.

30. The method of any of claims 1-29, wherein the amplification to generate a concatemer is carried out using rolling circle amplification (RCA).

31. The method of claim 30, wherein the RCA is performed using a circularized nucleic acid probe as a template and the reporter oligonucleotide as a primer.

32. The method of claim 30, wherein the RCA is performed using a circularized nucleic acid probe as a template and a second oligonucleotide as a primer.

33. The method of any of claims 1-32, wherein the concatemer is generated in situ.

34. The method of any of claims 1-33, wherein the concatemer is between about 1 and about 15 kilobases, between about 15 and about 25 kilobases, between about 25 and about 35 kilobases, between about 35 and about 45 kilobases, or between about 45 and about 50 kilobases in length.

35. The method of any of claims 1-34, wherein the concatemer forms a nanoball having a diameter between about 0.1 μm and about 3 μm.

36. The method of any of claims 3-24, wherein the detecting the concatemer or a portion thereof comprises determining a sequence of the barcode sequence or a complementary sequence thereof or an amplified sequence thereof.

37. The method of any of claims 3-25, wherein the detecting the concatemer or a portion thereof comprises in situ sequencing and/or in situ hybridization.

38. The method of claim 37, wherein the in situ sequencing comprises sequencing by ligation, sequencing by hybridization, sequencing by synthesis, and/or sequencing by binding.

39. The method of claim 37, wherein the in situ hybridization comprises sequential fluorescent in situ hybridization.

40. The method of any of claims 3-39, wherein the detecting the concatemer or a portion thereof comprises imaging the biological sample.

41. The method of claim 40, wherein the imaging comprises fluorescent microscopy.

42. The method of any of claims 6 and 8-41, wherein the reverse transcription of the mRNA is performed in the presence of a modified nucleotide.

43. The method of claim 42, wherein the modified nucleotide is selected from among a halogenated base, an azide-modified base, an aminoallyl dUTP, an octadiynyl dU, a thiol-modified base, a biotin-modified base, or a combination thereof.

44. The method of claim 42 or 43, further comprising crosslinking the modified nucleotide to the sample, a substrate, and/or a matrix, thereby crosslinking the cDNA to the sample, the substrate, and/or the matrix.

45. The method of any of claims 1-44, wherein the cassette further comprises an adapter sequence.

46. The method of any of claims 21-45, wherein the sequencing the tagged cDNAs or a portion thereof comprises direct sequencing or indirect sequencing.

47. The method of claim 46, further comprising, prior to the sequencing, ligating or adding a second adapter sequence to the tagged cDNA.

48. The method any of claims 21-47, wherein the tagged cDNA in the library or a portion thereof is amplified prior to the sequencing.

49. The method of any of claims 21-48, wherein the portion of the tagged cDNA that is sequenced comprises at least the barcode sequence and a portion of the target nucleic acid or complement thereof.

50. The method of any of claims 21-49, wherein the portion of the tagged cDNA that is sequenced comprises the spatial barcode sequence.

51. The method of any of claims 1-50, wherein the analyte-binding moiety is a protein, a peptide, an antibody or an epitope binding fragment thereof, a lipophilic moiety, a cell surface receptor binding molecule, a receptor ligand, a small molecule, a bi-specific antibody, a bi-specific T-cell engager, a T-cell receptor engager, a B-cell receptor engager, a pro-body, an aptamer, a monobody, an affimer, a darpin, and a protein scaffold, or any combination thereof.

52. The method of any of claims 1-51, wherein the analyte-binding moiety is an antibody or an epitope binding fragment thereof.

53. The method of any of claims 1-52, wherein the biological sample is a processed or cleared biological sample.

54. The method of any of claims 1-53, wherein the biological sample is a tissue sample.

55. The method of claim 54, wherein the tissue sample is a tissue slice between about 1 μm and about 50 μm in thickness.

56. The method of any of claims 1-55, wherein the biological sample is embedded in a hydrogel.

57. The method of any of claims 1-55, wherein the biological sample is not embedded in a hydrogel.

58. The method of any of claims 1-57, wherein the biological sample is fixed.

59. The method of any of claims 1-57, further comprising fixing the biological sample.

60. A kit comprising:
(a) a labeling agent comprising an analyte-binding moiety conjugated to a reporter oligonucleotide; and
(b) a nucleic acid probe comprising a cassette comprising (1) a restriction site, (2) a nucleic acid capture sequence, and (3) a barcode sequence, wherein the nucleic acid probe hybridizes to the reporter oligonucleotide.

61. The kit of claim 60, further comprising instructions for performing the method of any of claims 1-59.

62. The kit of claim 60 or 61, further comprising a reverse transcriptase.

63. The kit of any of claims 60-62, further comprising a restriction endonuclease.

64. The kit of claim 63, wherein the restriction endonuclease is selected from among DraI, MseI, TaqI-v2 and XbaI.

65. The kit of claim 63, wherein the restriction endonuclease is selected from among AcII, AgeI, BgIII, HindIII, SpeI, MluI, and PciI.

66. The kit of any of claims 60-65, further comprising a template-switch oligonucleotide (TSO).

67. A composition comprising:
(a) a labeling agent comprising an analyte-binding moiety conjugated to a reporter oligonucleotide;
(b) a nucleic acid probe comprising a cassette comprising (1) a restriction site, (2) a nucleic acid capture sequence, and (3) a barcode sequence, wherein the nucleic acid probe hybridizes to the reporter oligonucleotide; and
(c) a biological sample comprising a target nucleic acid.

68. The composition of claim 67, further comprising an amplification product of the nucleic acid probe that comprises a concatemer of monomer sequences, wherein each monomer sequence comprises sequences that are complementary to the cassette.
